(19) United States
(12) Patent Application Publication
(10) Pub. No.: US 2019/0233512 A1
(43) Pub. Date: Aug. 1, 2019

(54) ANTI-FOLATE RECEPTOR ANTIBODIES, COMPOSITIONS COMPRISING ANTI-FOLATE RECEPTOR ANTIBODIES AND METHODS OF MAKING AND USING ANTI-FOLATE RECEPTOR ANTIBODIES

(71) Applicant: SUTRO BIOPHARMA, INC., South San Francisco, CA (US)

(72) Inventors: Ryan STAFFORD, Emeryville, CA (US); Alice YAM, Tiburon, CA (US); Xiaofan LI, Fremont, CA (US); Robert HENNINGSON, Redwood City, CA (US); Sihong ZHOU, Foster City, CA (US); Heather STEPHENSON, San Jose, CA (US); Junhao YANG, Palo Alto, CA (US); Aaron SATO, Burlingame, CA (US)

(21) Appl. No.: 16/341,015

(22) PCT Filed: Oct. 11, 2017
(86) PCT No.: PCT/US2017/056223

§ 371 Date: Apr. 10, 2019

Related U.S. Application Data
(60) Provisional application No. 62/407,409, filed on Oct. 12, 2016.

(51) Int. Cl. C07K 16/28; A61K 49/00; A61P 35/00; A61K 51/10

(52) U.S. Cl. CPC C07K 16/28; C07K 2317/55; A61P 35/00; A61K 51/1027; C07K 2317/565; C07K 2317/52; C07K 2317/24; C07K 2317/21; C07K 2317/40; C07K 2317/622; C07K 2317/92; C07K 2317/76; C07K 2317/732; C07K 2317/734; A61K 49/0058

(57) ABSTRACT

The present disclosure relates to antibodies that selectively bind to folate receptor alpha (FOLR1) and its isoforms and homologs, and compositions comprising the antibodies. Also provided are methods of using the antibodies, such as therapeutic and diagnostic methods.

### FIELD OF THE INVENTION

The present disclosure generally relates to antibodies with binding specificity for folate receptor alpha (FOLR1) and compositions comprising the antibodies, including pharmaceutical compositions, diagnostic compositions, and kits. Also provided are methods of making anti-folate receptor antibodies, and methods of using anti-folate receptor antibodies, for example, for therapeutic purposes, diagnostic purposes, and research purposes.

### BACKGROUND

Folate receptors, or folate binding proteins (FBPs), include single chain glycoproteins that bind and contribute to the update of folates and other compounds in vivo. Elwood, 1989, J. Biol. Chem. 264:14893-14901. Certain folate receptors are single-chain glycoproteins with a high affinity binding site for folate and other compounds such as methotrexate. Elwood, p. 14893. The human FOLR1 gene encodes the adult folate receptor, a 30 kDa polypeptide with about 257 amino acids with three potential N-linked glycosylation sites. Elwood, p. 14893; Lacey et al., 1989, J. Clin. Invest. 84:715-720. Homologous genes and polypeptides have been identified in dozens of species.

The mature folate receptor glycoprotein has a size of about 42 kDa and has been observed to participate in the internalization of folates and antifolates into cells. Elwood et al., 1997, Biochemistry 36:1467-1478. Expression has been observed in human cerebellum and kidney cells, along with human cancer cell lines. Elwood et al., 1997, p. 1467. In addition to internalization of folate, a folate receptor has been shown to be a significant cofactor for cellular entry of viruses, particularly Marburg and Ebola viruses. Chan et al., 2001, Cell 106:117-126. Due to these internalization properties, the folate receptor has been proposed as a target for diagnostic and therapeutic agents. For instance, diagnostic and therapeutic agents have been linked to folate for internalization into cells expressing the folate receptor. See, e.g., Leamon, 2008, Curr. Opin. Investig. Drugs 9:1277-1286; Paulos et al., 2004, Adv. Drug Del. Rev. 56:1205-1217.

Folate receptor alpha (FolRα or FOLR1) is a glycosylphosphatidylinositol linked cell-surface glycoprotein that has high affinity for folates. Except for low levels in kidney and lung, most normal tissues do not express FOLR1, but high levels of FOLR1 have been found in serous and endometrioid epithelial ovarian cancer, endometrial adenocarcinoma, non-small cell lung carcinoma (NSCLC) of the adenocarcinoma subtype, and triple-negative breast cancer (TNBC). FOLR1 expression is maintained in metastatic foci and recurrent carcinomas in ovarian cancer patients, and FOLR1 expression has been observed after chemotherapy in epithelial ovarian and endometrial cancers. These properties, together with the highly restricted expression of FOLR1 on normal tissues, make FOLR1 a highly promising target for cancer therapy. As such, the folate receptor provides a potential target for diagnostics and therapeutics for cancers and inflammatory conditions. New antibodies are needed for specific binding and targeting of these folate receptors.

There is a need for improved methods of modulating the immune regulation of folate receptor alpha (FOLR1) and the downstream signaling processes activated by folate receptor alpha (FOLR1). Moreover, given the specific expression of folate receptor alpha (FOLR1) in cancer- and carcinoma-transformed cells and lower expression in non-cancer tissue, there is a need for improved therapeutics that can specifically target cells and tissues that overexpress folate receptor alpha (FOLR1).

### SUMMARY

Provided herein are antibodies that selectively bind folate receptor alpha (FOLR1). In some embodiments, the antibodies bind human folate receptor alpha. In some embodiments, the antibodies also bind homologs of human folate receptor alpha. In some aspects, the homologs include a cynomolgus monkey homolog and mouse homolog.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with one or more conservative amino acid substitutions.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-folate receptor antibodies provided herein. In some embodiments, the method is a method of treatment. In some embodiments, the method is a diagnostic method. In some embodiments, the method is an analytical method. In some embodiments, the method is a method of purifying and/or quantifying folate receptor alpha (FOLR1).

In some embodiments, the antibodies are used to treat a disease or condition. In some aspects, the disease or condition is selected from a cancer, autoimmune disease, and infection.

These and other embodiments of the invention along with many of its features are described in more detail in conjunction with the text below and attached figures.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Diibel (Eds.), Antibody Engineering vol. 2 (pp. 33-51). Springer-Verlag, Berlin Heidelberg.

FIGS. 2-4 provide alignments of the V_{H} sequences (SEQ ID NOs: 308-366) from the variant antibodies provided herein. CDRs according to Chothia are outlined/boxed, and CDRs according to Kabat are underlined.

FIG. 5 provides alignments of the V_{L} sequences (SEQ ID NOs: 367-369) from trastuzumab and the variant antibodies provided herein. CDRs according to Chothia are outlined/boxed, and CDRs according to Kabat are underlined.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, +5%, or +1%. In certain embodiments, the term “about” indicates the designated value±one standard deviation of that value.

The term “combinations thereof” includes every possible combination of elements to which the term refers to. For example, a sentence stating that “if” α₂ is A, then α₃ is not D; α₅ is not S; or α₆ is not S; or combinations thereof includes the following combinations when α₂ is A: (1) α₃ is not D; (2) α₅ is not S; (3) α₆ is not S; (4) α₃ is not D; α₅ is not S; and α₆ is not S; (5) α₃ is not D and α₅ is not S; (6) α₃ is not D and α₆ is not S; and (7) α₅ is not S and α₆ is not S.

The terms “folate receptor alpha” and “folate receptor 1” are used interchangeably herein. Folate receptor alpha is also known by synonyms, including FOLR1, FolRα, folate binding protein, FBP, adult folate binding protein, Folbp1, FR-alpha, FRα, KB cells FBP, and ovarian tumor-associated antigen MOv18, among others. Unless specified otherwise, the terms include any variants, isoforms and species homologs of human folate receptor alpha that are naturally expressed by cells, or that are expressed by cells transfected with a folate receptor alpha or FOLR1 gene. Folate receptor alpha proteins include, for example, human folate receptor alpha (SEQ ID NO: 1). In some embodiments, folate receptor alpha proteins include cynomolgus monkey folate receptor alpha (SEQ ID NO: 2). In some embodiments, folate receptor alpha proteins include murine folate receptor alpha (SEQ ID NO: 3).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (VI) and a heavy chain constant region (C_{H}). The heavy chain constant region typically comprises three domains, abbreviated C_{H}1, C_{H}2, and C_{H}3. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “folate receptor alpha antibody,” “anti-folate receptor alpha antibody,” “folate receptor alpha Ab,” “folate receptor alpha-specific antibody,” “anti-folate receptor alpha Ab,” “FOLR1 antibody,” “FolRα antibody,” “anti-FOLR1 antibody,” “anti-FolRα antibody,” “FOLR1 Ab,” “FolRα Ab,” “FOLR1-specific antibody,” “FolRα-specific antibody,” “anti-FolRα Ab,” or “anti-FOLR1 Ab” is an antibody, as described herein, which binds specifically to folate receptor alpha or FOLR1. In some embodiments, the antibody binds the extracellular domain of folate receptor alpha (FOLR1).

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat and Chothia
numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

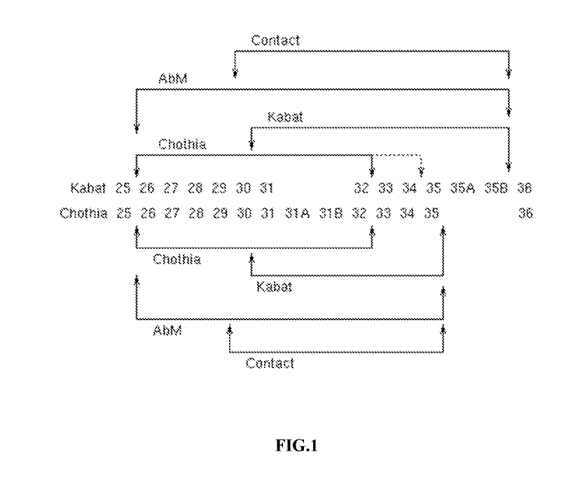

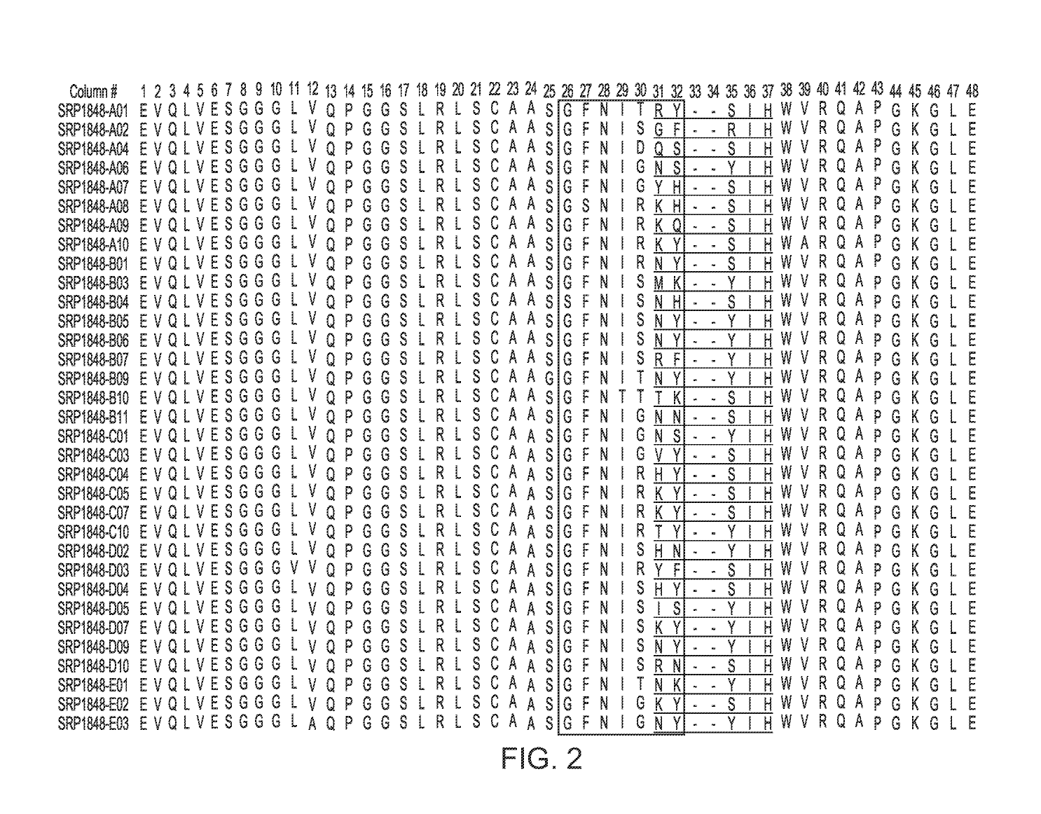

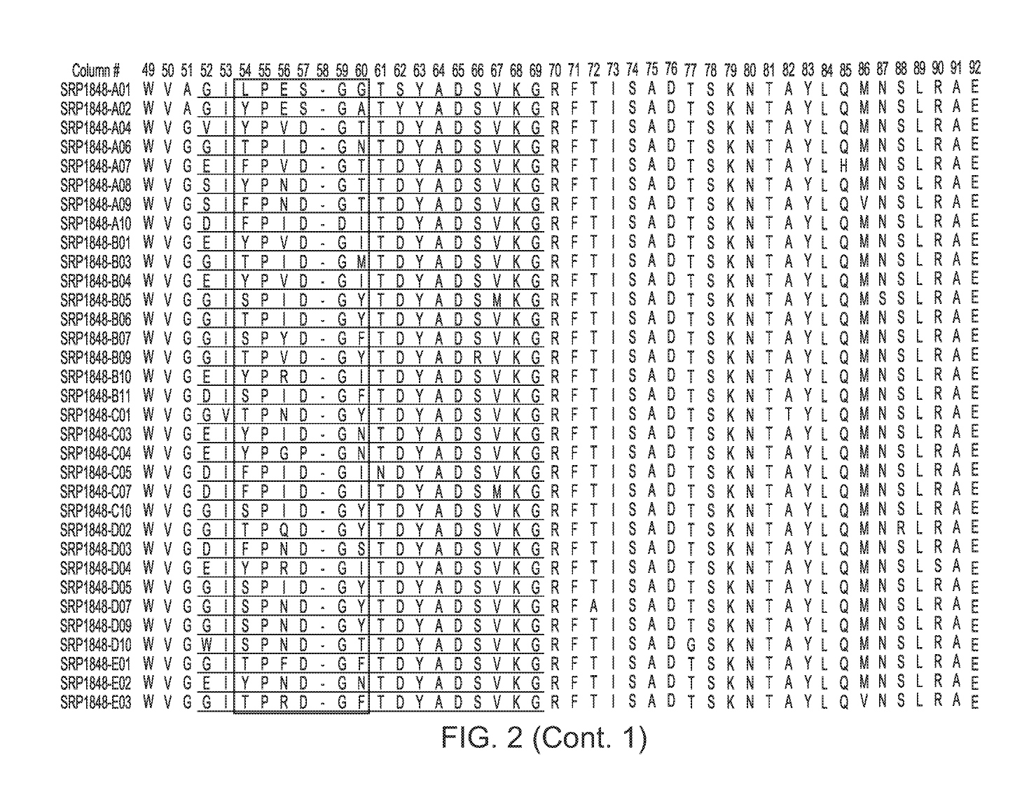

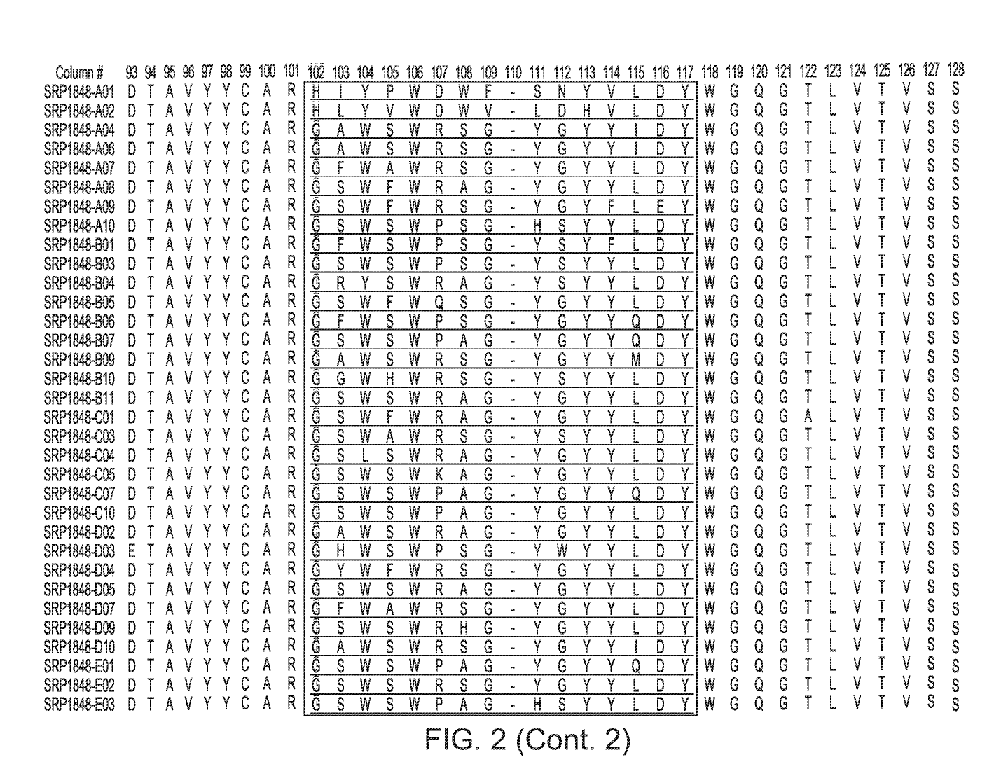

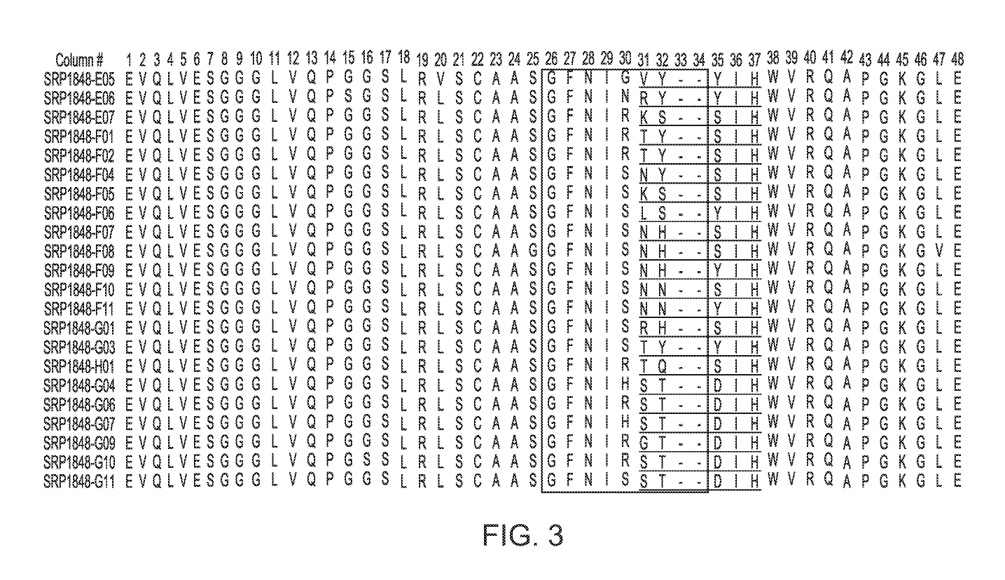

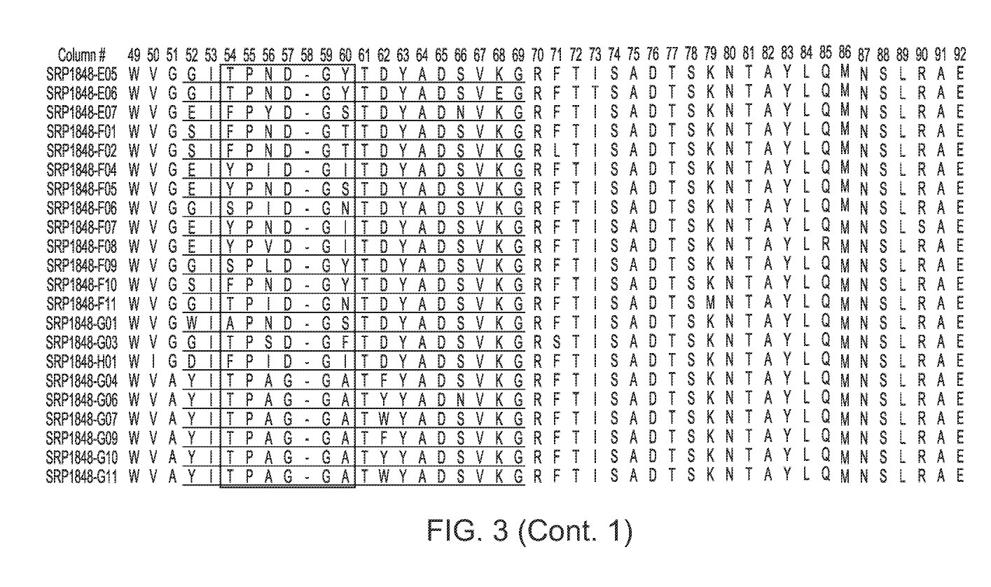

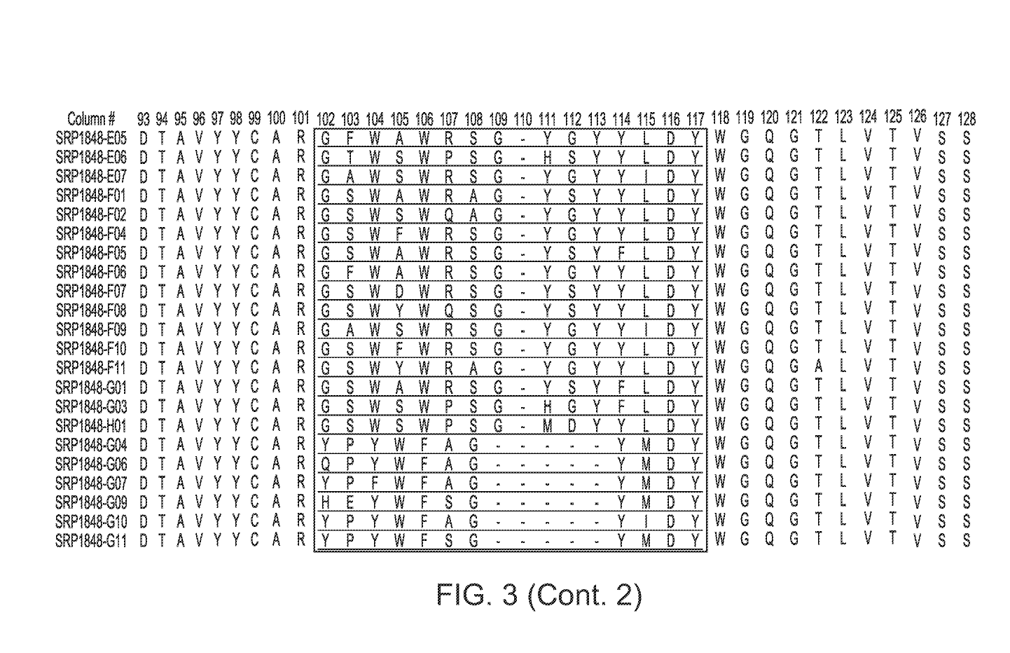

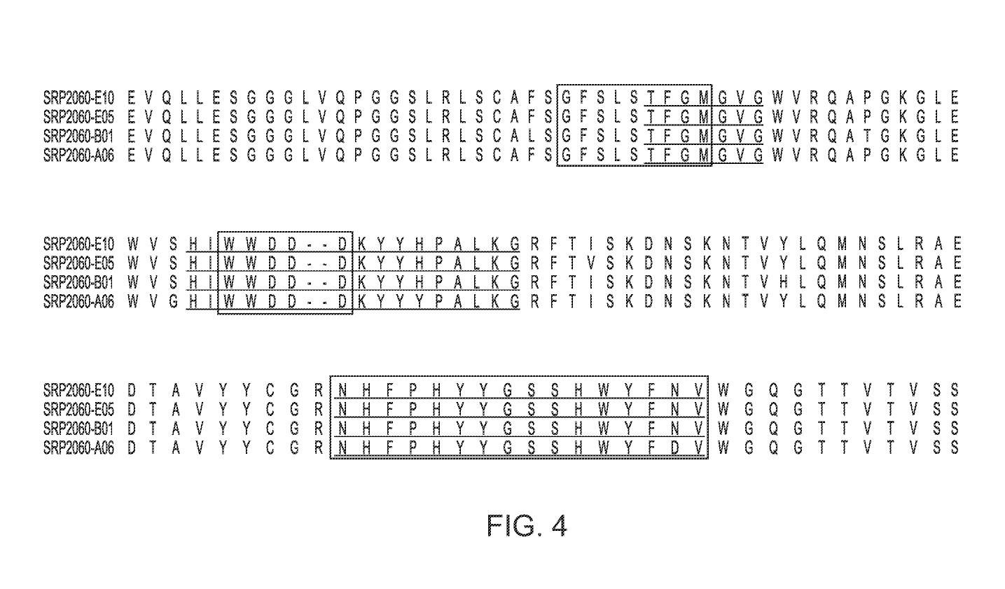

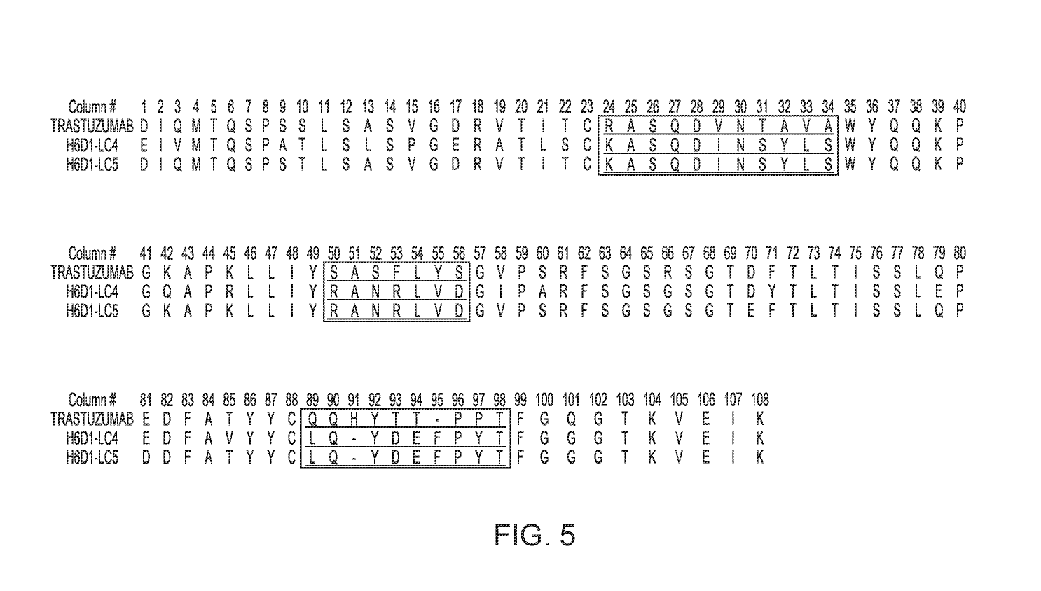

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at http://www.bioinf.org.uk/abs/abnum/, and described in Abhinandan and Martin, Immunology, 2008, 45:3832-3839, incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H}1) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 377. In some embodiments, the linker is SEQ ID NO: 378. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 370, or a portion thereof. SEQ ID NO: 370 provides the sequence of C_{H}1, C_{H}2, and C_{H3} of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., folate receptor alpha, or FOLR1). In one exemplary assay, FOLR1 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of folate receptor alpha (FOLR1) with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, R, T, V, |
| | W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, |
| | P, and T |
| Flexible Residues | Q, T, K, S, G, P, D, E, and R |

TABLE 3
Additional selected groups of amino acids that are considered
conservative substitutions for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered
conservative substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder. In some embodiments, a therapeutically effective amount or effective amount refers to an amount of an antibody or composition that when administered to a subject is effective to prevent or ameliorate a disease or the progression of the disease, or result in amelioration of symptoms.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

### 2. Antibodies

Provided herein are antibodies that selectively bind human folate receptor alpha. In some aspects, the antibody selectively binds to the extracellular domain of human folate receptor alpha (human FOLR1).

In some embodiments, the antibody binds to a homolog of human FOLR1. In some aspects, the antibody binds to a homolog of human FOLR1 from a species selected from monkeys, mice, dogs, cats, rats, cows, horses, goats and sheep. In some aspects, the homolog is a cynomolgus monkey homolog. In some aspects, the homolog is a mouse or murine analog.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with a conservative amino acid substitution.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 13, 14, 15, 16, or 17 residues in length.

In some aspects, the Kabat/Chothia CDR-L1 of the antibody is 11, 12, 13, 14, 15, 16, 17, or 18 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

2.1 CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs.: 308-366.

In some embodiments, the antibody comprises a CDR-H3 sequence comprising, consisting of, or consisting essentially of a sequence selected from SEQ ID NOs.: 240-298. In some aspects, the antibody comprises a CDR-H3 sequence comprising, consisting of, or consisting essentially of SEQ ID NO: 240. In some aspects, the antibody comprises a CDR-H3 sequence comprising, consisting of, or consisting essentially of SEQ ID NO: 241. In some aspects, the antibody comprises a CDR-H3 sequence comprising, consisting of, or consisting essentially of SEQ ID NO: 242. In some aspects, the antibody comprises a CDR-H3 sequence comprising, consisting of, or consisting essentially of SEQ ID NO: 243. In some aspects, the antibody comprises a CDR-H3 sequence comprising, consisting of, or consisting essentially of SEQ ID NO: 244. In some aspects, the antibody comprises a CDR-H3 sequence comprising, consisting of, or consisting essentially of SEQ ID NO: 245. In some aspects, the antibody comprises a CDR-H3 sequence comprising, consisting of, or consisting essentially of SEQ ID NO: 246. In some aspects, the antibody comprises a CDR-H3 sequence comprising, consisting of, or consisting essentially of SEQ ID NO: 247. In some aspects, the antibody comprises a CDR-H3 sequence comprising, consisting of, or consisting essentially of SEQ ID NO: 248. In some aspects, the antibody comprises a CDR-H3 sequence comprising, consisting of, or consisting essentially of SEQ ID NO: 249. In some aspects, the antibody comprises a CDR-H3 sequence comprising, consisting of, or consisting essentially of SEQ ID NO: 250. In some aspects, the antibody comprises a CDR-H3 sequence comprising, consisting of, or consisting essentially of SEQ ID NO: 251. In some aspects, the antibody comprises a CDR-H3 sequence comprising, consisting of, or consisting essentially of SEQ ID NO: 252. In some aspects, the antibody comprises a CDR-H3 sequence comprising, consisting of, or consisting essentially of SEQ ID NO: 253. In some aspects, the antibody comprises a CDR-H3 sequence comprising, consisting of, or consisting essentially of SEQ ID NO: 254. In some aspects, the antibody comprises a CDR-H3 sequence comprising, consisting of, or consisting essentially of SEQ ID NO: 255. In some aspects, the antibody comprises a CDR-H3 sequence comprising, consisting of, or consisting essentially of SEQ ID NO: 256. In some aspects, the antibody comprises a CDR-H3 sequence comprising, consisting of, or consisting essentially of SEQ ID NO: 257. In some aspects, the antibody comprises a CDR-H3 sequence comprising, consisting of, or consisting essentially of SEQ ID NO: 258. In some aspects, the antibody comprises a CDR-H3 sequence comprising, consisting of, or consisting essentially of SEQ ID NO: 259. In some aspects, the antibody comprises a CDR-H3 sequence comprising, consisting of, or consisting essentially of SEQ ID NO: 260. In some aspects, the antibody comprises a CDR-H3 sequence comprising, consisting of, or consisting essentially of SEQ ID NO: 261. In some aspects, the antibody comprises a CDR-H3 sequence comprising, consisting of, or consisting essentially of SEQ ID NO: 262. In some aspects, the antibody comprises a CDR-H3 sequence comprising, consisting of, or consisting essentially of SEQ ID NO: 263. In some aspects, the antibody comprises a CDR-H3 sequence comprising, consisting of, or consisting essentially of SEQ ID NO: 264. In some aspects, the antibody comprises a CDR-H3 sequence comprising, consisting of, or consisting essentially of SEQ ID NO: 265. In some aspects, the antibody comprises a CDR-H3 sequence comprising, consisting of, or consisting essentially of SEQ ID NO: 266. In some aspects, the antibody comprises a CDR-H3 sequence comprising, consisting of, or consisting essentially of SEQ ID NO: 267. In some aspects, the antibody comprises a CDR-H3 sequence comprising, consisting of, or consisting essentially of SEQ ID NO: 268. In some aspects, the antibody comprises a CDR-H3 sequence comprising, consisting of, or consisting essentially of SEQ ID NO: 269. In some aspects, the antibody comprises a CDR-H3 sequence comprising, consisting of, or consisting essentially of SEQ ID NO: 270. In some aspects, the antibody comprises a CDR-H3 sequence comprising, consisting of, or consisting essentially of SEQ ID NO: 271. In some aspects, the antibody comprises a CDR-H3 sequence comprising, consisting of, or consisting essentially of SEQ ID NO: 272. In some aspects, the antibody comprises a CDR-H3 sequence comprising, consisting of, or consisting essentially of SEQ ID NO: 273. In some aspects, the antibody comprises a CDR-H3 sequence comprising, consisting of, or consisting essentially of SEQ ID NO: 274. In some aspects, the antibody comprises a CDR-H3 sequence comprising, consisting of, or consisting essentially of SEQ ID NO: 275. In some aspects, the antibody comprises a CDR-H3 sequence comprising, consisting of, or consisting essentially of SEQ ID NO: 276. In some aspects, the antibody comprises a CDR-H3 sequence comprising, consisting of, or consisting essentially of SEQ ID NO: 277. In some aspects, the antibody comprises a CDR-H3 sequence comprising, consisting of, or consisting essentially of SEQ ID NO: 278. In some aspects, the antibody comprises a CDR-H3 sequence comprising, consisting of, or consisting essentially of SEQ ID NO: 279. In some aspects, the antibody comprises a CDR-H3 sequence comprising, consisting of, or consisting essentially of SEQ ID NO: 280. In some aspects, the antibody comprises a CDR-H3 sequence comprising, consisting of, or consisting essentially of SEQ ID NO: 281. In some aspects, the antibody comprises a CDR-H3 sequence comprising, consisting of, or consisting essentially of SEQ ID NO: 282. In some aspects, the antibody comprises a CDR-H3 sequence comprising, consisting of, or consisting essentially of SEQ ID NO: 283. In some aspects, the antibody comprises a CDR-H3 sequence comprising, consisting of, or consisting essentially of SEQ ID NO: 284. In some aspects, the antibody comprises a CDR-H3 sequence comprising, consisting of, or consisting essentially of SEQ ID NO: 285. In some aspects, the antibody comprises a CDR-H3 sequence comprising, consisting of, or consisting essentially of SEQ ID NO: 286. In some aspects, the antibody comprises a CDR-H3 sequence comprising, consisting of, or consisting essentially of SEQ ID NO: 287. In some aspects, the antibody comprises a CDR-H3 sequence comprising, consisting of, or consisting essentially of SEQ ID NO: 288. In some aspects, the antibody comprises a CDR-H3 sequence comprising, consisting of, or consisting essentially of SEQ ID NO: 289. In some aspects, the antibody comprises a CDR-H3 sequence comprising, consisting of, or consisting essentially of SEQ ID NO: 290. In some aspects, the antibody comprises a CDR-H3 sequence comprising, consisting of, or consisting essentially of SEQ ID NO: 291. In some aspects, the antibody comprises a CDR-H3 sequence comprising, consisting of, or consisting essentially of SEQ ID NO: 292. In some aspects, the antibody comprises a CDR-H3 sequence comprising, consisting of, or consisting essentially of SEQ ID NO: 293. In some aspects, the antibody comprises a CDR-H3 sequence comprising, consisting of, or consisting essentially of SEQ ID NO: 294. In some aspects, the antibody comprises a CDR-H3 sequence comprising, consisting of, or consisting essentially of SEQ ID NO: 295. In some aspects, the antibody comprises a CDR-H3 sequence comprising, consisting of, or consisting essentially of SEQ ID NO: 296. In some aspects, the antibody comprises a CDR-H3 sequence comprising, consisting of, or consisting essentially of SEQ ID NO: 297. In some aspects, the antibody comprises a CDR-H3 sequence comprising, consisting of, or consisting essentially of SEQ ID NO: 298.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

2.2 V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 308-366.

2.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

2.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 308-366.

In some embodiments, the antibody comprises a V_{H} sequence comprising a Kabat CDR-H3 sequence comprising, consisting of, or consisting essentially of a sequence selected from SEQ ID NOs.: 240-298. In some aspects, the antibody comprises a V_{H} sequence comprising a Kabat CDR-H3 sequence comprising, consisting of, or consisting essentially of SEQ ID NO: 240. In some aspects, the antibody comprises a V_{H} sequence comprising a Kabat CDR-H3 sequence comprising, consisting of, or consisting essentially of SEQ ID NO: 241. In some aspects, the antibody comprises a V_{H} sequence comprising a Kabat CDR-H3 sequence comprising, consisting of, or consisting essentially of SEQ ID NO: 242. In some aspects, the antibody comprises a V_{H} sequence comprising a Kabat CDR-H3 sequence comprising, consisting of, or consisting essentially of SEQ ID NO: 243. In some aspects, the antibody comprises a V_{H} sequence comprising a Kabat CDR-H3 sequence comprising, consisting of, or consisting essentially of SEQ ID NO: 244. In some aspects, the antibody comprises a V_{H} sequence comprising a Kabat CDR-H3 sequence comprising, consisting of, or consisting essentially of SEQ ID NO: 245. In some aspects, the antibody comprises a V_{H} sequence comprising a Kabat CDR-H3 sequence comprising, consisting of, or consisting essentially of SEQ ID NO: 246. In some aspects, the antibody comprises a V_{H} sequence comprising a Kabat CDR-H3 sequence comprising, consisting of, or consisting essentially of SEQ ID NO: 247. In some aspects, the antibody comprises a V_{H} sequence comprising a Kabat CDR-H3 sequence comprising, consisting of, or consisting essentially of SEQ ID NO: 248. In some aspects, the antibody comprises a V_{H} sequence comprising a Kabat CDR-H3 sequence comprising, consisting of, or consisting essentially of SEQ ID NO: 249. In some aspects, the antibody comprises a V_{H} sequence comprising a Kabat CDR-H3 sequence comprising, consisting of, or consisting essentially of SEQ ID NO: 250. In some aspects, the antibody comprises a V_{H} sequence comprising a Kabat CDR-H3 sequence comprising, consisting of, or consisting essentially of SEQ ID NO: 251. In some aspects, the antibody comprises a V_{H} sequence comprising a Kabat CDR-H3 sequence comprising, consisting of, or consisting essentially of SEQ ID NO: 252. In some aspects, the antibody comprises a V_{H} sequence comprising a Kabat CDR-H3 sequence comprising, consisting of, or consisting essentially of SEQ ID NO: 253. In some aspects, the antibody comprises a V_{H} sequence comprising a Kabat CDR-H3 sequence comprising, consisting of, or consisting essentially of SEQ ID NO: 254. In some aspects, the antibody comprises a V_{H} sequence comprising a Kabat CDR-H3 sequence comprising, consisting of, or consisting essentially of SEQ ID NO: 255. In some aspects, the antibody comprises a V_{H} sequence comprising a Kabat CDR-H3 sequence comprising, consisting of, or consisting essentially of SEQ ID NO: 256. In some aspects, the antibody comprises a V_{H} sequence comprising a Kabat CDR-H3 sequence comprising, consisting of, or consisting essentially of SEQ ID NO: 257. In some aspects, the antibody comprises a V_{H} sequence comprising a Kabat CDR-H3 sequence comprising, consisting of, or consisting essentially of SEQ ID NO: 258. In some aspects, the antibody comprises a V_{H} sequence comprising a Kabat CDR-H3 sequence comprising, consisting of, or consisting essentially of SEQ ID NO: 259. In some aspects, the antibody comprises a V_{H} sequence comprising a Kabat CDR-H3 sequence comprising, consisting of, or consisting essentially of SEQ ID NO: 260. In some aspects, the antibody comprises a V_{H} sequence comprising a Kabat CDR-H3 sequence comprising, consisting of, or consisting essentially of SEQ ID NO: 261. In some aspects, the antibody comprises a V_{H} sequence comprising a Kabat CDR-H3 sequence comprising, consisting of, or consisting essentially of SEQ ID NO: 262. In some aspects, the antibody comprises a V_{H} sequence comprising a Kabat CDR-H3 sequence comprising, consisting of, or consisting essentially of SEQ ID NO: 263. In some aspects, the antibody comprises a V_{H} sequence comprising a Kabat CDR-H3 sequence comprising, consisting of, or consisting essentially of SEQ ID NO: 264. In some aspects, the antibody comprises a V_{H} sequence comprising a Kabat CDR-H3 sequence comprising, consisting of, or consisting essentially of SEQ ID NO: 265. In some aspects, the antibody comprises a V_{H} sequence comprising a Kabat CDR-H3 sequence comprising, consisting of, or consisting essentially of SEQ ID NO: 266. In some aspects, the antibody comprises a V_{H} sequence comprising a Kabat CDR-H3 sequence comprising, consisting of, or consisting essentially of SEQ ID NO: 267. In some aspects, the antibody comprises a V_{H} sequence comprising a Kabat CDR-H3 sequence comprising, consisting of, or consisting essentially of SEQ ID NO: 268. In some aspects, the antibody comprises a V_{H} sequence comprising a Kabat CDR-H3 sequence comprising, consisting of, or consisting essentially of SEQ ID NO: 269. In some aspects, the antibody comprises a V_{H} sequence comprising a Kabat CDR-H3 sequence comprising, consisting of, or consisting essentially of SEQ ID NO: 270. In some aspects, the antibody comprises a V_{H} sequence comprising a Kabat CDR-H3 sequence comprising, consisting of, or consisting essentially of SEQ ID NO: 271. In some aspects, the antibody comprises a V_{H} sequence comprising a Kabat CDR-H3 sequence comprising, consisting of, or consisting essentially of SEQ ID NO: 272. In some aspects, the antibody comprises a V_{H} sequence comprising a Kabat CDR-H3 sequence comprising, consisting of, or consisting essentially of SEQ ID NO: 273. In some aspects, the antibody comprises a V_{H} sequence comprising a Kabat CDR-H3 sequence comprising, consisting of, or consisting essentially of SEQ ID NO: 274. In some aspects, the antibody comprises a V_{H} sequence comprising a Kabat CDR-H3 sequence comprising, consisting of, or consisting essentially of SEQ ID NO: 275. In some aspects, the antibody comprises a V_{H} sequence comprising a Kabat CDR-H3 sequence comprising, consisting of, or consisting essentially of SEQ ID NO: 276. In some aspects, the antibody comprises a V_{H} sequence comprising a Kabat CDR-H3 sequence comprising, consisting of, or consisting essentially of SEQ ID NO: 277. In some aspects, the antibody comprises a V_{H} sequence comprising a Kabat CDR-H3 sequence comprising, consisting of, or consisting essentially of SEQ ID NO: 278. In some aspects, the antibody comprises a V_{H} sequence comprising a Kabat CDR-H3 sequence comprising, consisting of, or consisting essentially of SEQ ID NO: 279. In some aspects, the antibody comprises a V_{H} sequence comprising a Kabat CDR-H3 sequence comprising, consisting of, or consisting essentially of SEQ ID NO: 280. In some aspects, the antibody comprises a V_{H} sequence comprising a Kabat CDR-H3 sequence comprising, consisting of, or consisting essentially of SEQ ID NO: 281. In some aspects, the antibody comprises a V_{H} sequence comprising a Kabat CDR-H3 sequence comprising, consisting of, or consisting essentially of SEQ ID NO: 282. In some aspects, the antibody comprises a V_{H} sequence comprising a Kabat CDR-H3 sequence comprising, consisting of, or consisting essentially of SEQ ID NO: 283. In some aspects, the antibody comprises a V_{H} sequence comprising a Kabat CDR-H3 sequence comprising, consisting of, or consisting essentially of SEQ ID NO: 284. In some aspects, the antibody comprises a V_{H} sequence comprising a Kabat CDR-H3 sequence comprising, consisting of, or consisting essentially of SEQ ID NO: 285. In some aspects, the antibody comprises a V_{H} sequence comprising a Kabat CDR-H3 sequence comprising, consisting of, or consisting essentially of SEQ ID NO: 286. In some aspects, the antibody comprises a V_{H} sequence comprising a Kabat CDR-H3 sequence comprising, consisting of, or consisting essentially of SEQ ID NO: 287. In some aspects, the antibody comprises a V_{H} sequence comprising a Kabat CDR-H3 sequence comprising, consisting of, or consisting essentially of SEQ ID NO: 288. In some aspects, the antibody comprises a V_{H} sequence comprising a Kabat CDR-H3 sequence comprising, consisting of, or consisting essentially of SEQ ID NO: 289. In some aspects, the antibody comprises a V_{H} sequence comprising a Kabat CDR-H3 sequence comprising, consisting of, or consisting essentially of SEQ ID NO: 290. In some aspects, the antibody comprises a V_{H} sequence comprising a Kabat CDR-H3 sequence comprising, consisting of, or consisting essentially of SEQ ID NO: 291. In some aspects, the antibody comprises a V_{H} sequence comprising a Kabat CDR-H3 sequence comprising, consisting of, or consisting essentially of SEQ ID NO: 292. In some aspects, the antibody comprises a V_{H} sequence comprising a Kabat CDR-H3 sequence comprising, consisting of, or consisting essentially of SEQ ID NO: 293. In some aspects, the antibody comprises a V_{H} sequence comprising a Kabat CDR-H3 sequence comprising, consisting of, or consisting essentially of SEQ ID NO: 294. In some aspects, the antibody comprises a V_{H} sequence comprising a Kabat CDR-H3 sequence comprising, consisting of, or consisting essentially of SEQ ID NO: 295. In some aspects, the antibody comprises a V_{H} sequence comprising a Kabat CDR-H3 sequence comprising, consisting of, or consisting essentially of SEQ ID NO: 296. In some aspects, the antibody comprises a V_{H} sequence comprising a Kabat CDR-H3 sequence comprising, consisting of, or consisting essentially of SEQ ID NO: 297. In some aspects, the antibody comprises a V_{H} sequence comprising a Kabat CDR-H3 sequence comprising, consisting of, or consisting essentially of SEQ ID NO: 298.

2.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 308-366.

In some embodiments, the antibody comprises a V_{H} sequence comprising a Kabat CDR-H2 sequence comprising, consisting of, or consisting essentially of a sequence selected from SEQ ID NOs: 181-239. In some aspects, the antibody comprises a V_{H} sequence comprising a Kabat CDR-H2 sequence comprising, consisting of, or consisting essentially of SEQ ID NO: 181. In some aspects, the antibody comprises a V_{H} sequence comprising a Kabat CDR-H2 sequence comprising, consisting of, or consisting essentially of SEQ ID NO: 182. In some aspects, the antibody comprises a V_{H} sequence comprising a Kabat CDR-H2 sequence comprising, consisting of, or consisting essentially of SEQ ID NO: 183. In some aspects, the antibody comprises a V_{H} sequence comprising a Kabat CDR-H2 sequence comprising, consisting of, or consisting essentially of SEQ ID NO: 184. In some aspects, the antibody comprises a V_{H} sequence comprising a Kabat CDR-H2 sequence comprising, consisting of, or consisting essentially of SEQ ID NO: 185. In some aspects, the antibody comprises a V_{H} sequence comprising a Kabat CDR-H2 sequence comprising, consisting of, or consisting essentially of SEQ ID NO: 186. In some aspects, the antibody comprises a V_{H} sequence comprising a Kabat CDR-H2 sequence comprising, consisting of, or consisting essentially of SEQ ID NO: 187. In some aspects, the antibody comprises a V_{H} sequence comprising a Kabat CDR-H2 sequence comprising, consisting of, or consisting essentially of SEQ ID NO: 188. In some aspects, the antibody comprises a V_{H} sequence comprising a Kabat CDR-H2 sequence comprising, consisting of, or consisting essentially of SEQ ID NO: 189. In some aspects, the antibody comprises a V_{H} sequence comprising a Kabat CDR-H2 sequence comprising, consisting of, or consisting essentially of SEQ ID NO: 190. In some aspects, the antibody comprises a V_{H} sequence comprising a Kabat CDR-H2 sequence comprising, consisting of, or consisting essentially of SEQ ID NO: 191. In some aspects, the antibody comprises a V_{H} sequence comprising a Kabat CDR-H2 sequence comprising, consisting of, or consisting essentially of SEQ ID NO: 192. In some aspects, the antibody comprises a V_{H} sequence comprising a Kabat CDR-H2 sequence comprising, consisting of, or consisting essentially of SEQ ID NO: 193. In some aspects, the antibody comprises a V_{H} sequence comprising a Kabat CDR-H2 sequence comprising, consisting of, or consisting essentially of SEQ ID NO: 194. In some aspects, the antibody comprises a V_{H} sequence comprising a Kabat CDR-H2 sequence comprising, consisting of, or consisting essentially of SEQ ID NO: 195. In some aspects, the antibody comprises a V_{H} sequence comprising a Kabat CDR-H2 sequence comprising, consisting of, or consisting essentially of SEQ ID NO: 196. In some aspects, the antibody comprises a V_{H} sequence comprising a Kabat CDR-H2 sequence comprising, consisting of, or consisting essentially of SEQ ID NO: 197. In some aspects, the antibody comprises a V_{H} sequence comprising a Kabat CDR-H2 sequence comprising, consisting of, or consisting essentially of SEQ ID NO: 198. In some aspects, the antibody comprises a V_{H} sequence comprising a Kabat CDR-H2 sequence comprising, consisting of, or consisting essentially of SEQ ID NO: 199. In some aspects, the antibody comprises a V_{H} sequence comprising a Kabat CDR-H2 sequence comprising, consisting of, or consisting essentially of SEQ ID NO: 200. In some aspects, the antibody comprises a V_{H} sequence comprising a Kabat CDR-H2 sequence comprising, consisting of, or consisting essentially of SEQ ID NO: 201. In some aspects, the antibody comprises a V_{H} sequence comprising a Kabat CDR-H2 sequence comprising, consisting of, or consisting essentially of SEQ ID NO: 202. In some aspects, the antibody comprises a V_{H} sequence comprising a Kabat CDR-H2 sequence comprising, consisting of, or consisting essentially of SEQ ID NO: 203. In some aspects, the antibody comprises a V_{H} sequence comprising a Kabat CDR-H2 sequence comprising, consisting of, or consisting essentially of SEQ ID NO: 204. In some aspects, the antibody comprises a V_{H} sequence comprising a Kabat CDR-H2 sequence comprising, consisting of, or consisting essentially of SEQ ID NO: 205. In some aspects, the antibody comprises a V_{H} sequence comprising a Kabat CDR-H2 sequence comprising, consisting of, or consisting essentially of SEQ ID NO: 206. In some aspects, the antibody comprises a V_{H} sequence comprising a Kabat CDR-H2 sequence comprising, consisting of, or consisting essentially of SEQ ID NO: 207. In some aspects, the antibody comprises a V_{H} sequence comprising a Kabat CDR-H2 sequence comprising, consisting of, or consisting essentially of SEQ ID NO: 208. In some aspects, the antibody comprises a V_{H} sequence comprising a Kabat CDR-H2 sequence comprising, consisting of, or consisting essentially of SEQ ID NO: 209. In some aspects, the antibody comprises a V_{H} sequence comprising a Kabat CDR-H2 sequence comprising, consisting of, or consisting essentially of SEQ ID NO: 210. In some aspects, the antibody comprises a V_{H} sequence comprising a Kabat CDR-H2 sequence comprising, consisting of, or consisting essentially of SEQ ID NO: 211. In some aspects, the antibody comprises a V_{H} sequence comprising a Kabat CDR-H2 sequence comprising, consisting of, or consisting essentially of SEQ ID NO: 212. In some aspects, the antibody comprises a V_{H} sequence comprising a Kabat CDR-H2 sequence comprising, consisting of, or consisting essentially of SEQ ID NO: 213. In some aspects, the antibody comprises a V_{H} sequence comprising a Kabat CDR-H2 sequence comprising, consisting of, or consisting essentially of SEQ ID NO: 214. In some aspects, the antibody comprises a V_{H} sequence comprising a Kabat CDR-H2 sequence comprising, consisting of, or consisting essentially of SEQ ID NO: 215. In some aspects, the antibody comprises a V_{H} sequence comprising a Kabat CDR-H2 sequence comprising, consisting of, or consisting essentially of SEQ ID NO: 216. In some aspects, the antibody comprises a V_{H} sequence comprising a Kabat CDR-H2 sequence comprising, consisting of, or consisting essentially of SEQ ID NO: 217. In some aspects, the antibody comprises a V_{H} sequence comprising a Kabat CDR-H2 sequence comprising, consisting of, or consisting essentially of SEQ ID NO: 218. In some aspects, the antibody comprises a V_{H} sequence comprising a Kabat CDR-H2 sequence comprising, consisting of, or consisting essentially of SEQ ID NO: 219. In some aspects, the antibody comprises a V_{H} sequence comprising a Kabat CDR-H2 sequence comprising, consisting of, or consisting essentially of SEQ ID NO: 220. In some aspects, the antibody comprises a V_{H} sequence comprising a Kabat CDR-H2 sequence comprising, consisting of, or consisting essentially of SEQ ID NO: 221. In some aspects, the antibody comprises a V_{H} sequence comprising a Kabat CDR-H2 sequence comprising, consisting of, or consisting essentially of SEQ ID NO: 222. In some aspects, the antibody comprises a V_{H} sequence comprising a Kabat CDR-H2 sequence comprising, consisting of, or consisting essentially of SEQ ID NO: 223. In some aspects, the antibody comprises a V_{H} sequence comprising a Kabat CDR-H2 sequence comprising, consisting of, or consisting essentially of SEQ ID NO: 224. In some aspects, the antibody comprises a V_{H} sequence comprising a Kabat CDR-H2 sequence comprising, consisting of, or consisting essentially of SEQ ID NO: 225. In some aspects, the antibody comprises a V_{H} sequence comprising a Kabat CDR-H2 sequence comprising, consisting of, or consisting essentially of SEQ ID NO: 226. In some aspects, the antibody comprises a V_{H} sequence comprising a Kabat CDR-H2 sequence comprising, consisting of, or consisting essentially of SEQ ID NO: 227. In some aspects, the antibody comprises a V_{H} sequence comprising a Kabat CDR-H2 sequence comprising, consisting of, or consisting essentially of SEQ ID NO: 228. In some aspects, the antibody comprises a V_{H} sequence comprising a Kabat CDR-H2 sequence comprising, consisting of, or consisting essentially of SEQ ID NO: 229. In some aspects, the antibody comprises a V_{H} sequence comprising a Kabat CDR-H2 sequence comprising, consisting of, or consisting essentially of SEQ ID NO: 230. In some aspects, the antibody comprises a V_{H} sequence comprising a Kabat CDR-H2 sequence comprising, consisting of, or consisting essentially of SEQ ID NO: 231. In some aspects, the antibody comprises a V_{H} sequence comprising a Kabat CDR-H2 sequence comprising, consisting of, or consisting essentially of SEQ ID NO: 232. In some aspects, the antibody comprises a V_{H} sequence comprising a Kabat CDR-H2 sequence comprising, consisting of, or consisting essentially of SEQ ID NO: 233. In some aspects, the antibody comprises a V_{H} sequence comprising a Kabat CDR-H2 sequence comprising, consisting of, or consisting essentially of SEQ ID NO: 234. In some aspects, the antibody comprises a V_{H} sequence comprising a Kabat CDR-H2 sequence comprising, consisting of, or consisting essentially of SEQ ID NO: 235. In some aspects, the antibody comprises a V_{H} sequence comprising a Kabat CDR-H2 sequence comprising, consisting of, or consisting essentially of SEQ ID NO: 236. In some aspects, the antibody comprises a V_{H} sequence comprising a Kabat CDR-H2 sequence comprising, consisting of, or consisting essentially of SEQ ID NO: 237. In some aspects, the antibody comprises a V_{H} sequence comprising a Kabat CDR-H2 sequence comprising, consisting of, or consisting essentially of SEQ ID NO: 238. In some aspects, the antibody comprises a V_{H} sequence comprising a Kabat CDR-H2 sequence comprising, consisting of, or consisting essentially of SEQ ID NO: 239.

2.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 308-366.

In some embodiments, the antibody comprises a V_{H} sequence comprising a Kabat CDR-H1 sequence comprising, consisting of, or consisting essentially of a sequence selected from SEQ ID NOs: 63-121. In some aspects, the antibody comprises a V_{H} sequence comprising a Kabat CDR-H1 sequence comprising, consisting of, or consisting essentially of SEQ ID NO: 63. In some aspects, the antibody comprises a V_{H} sequence comprising a Kabat CDR-H1 sequence comprising, consisting of, or consisting essentially of SEQ ID NO: 64. In some aspects, the antibody comprises a V_{H} sequence comprising a Kabat CDR-H1 sequence comprising, consisting of, or consisting essentially of SEQ ID NO: 65. In some aspects, the antibody comprises a V_{H} sequence comprising a Kabat CDR-H1 sequence comprising, consisting of, or consisting essentially of SEQ ID NO: 66. In some aspects, the antibody comprises a V_{H} sequence comprising a Kabat CDR-H1 sequence comprising, consisting of, or consisting essentially of SEQ ID NO: 67. In some aspects, the antibody comprises a V_{H} sequence comprising a Kabat CDR-H1 sequence comprising, consisting of, or consisting essentially of SEQ ID NO: 68. In some aspects, the antibody comprises a V_{H} sequence comprising a Kabat CDR-H1 sequence comprising, consisting of, or consisting essentially of SEQ ID NO: 69. In some aspects, the antibody comprises a V_{H} sequence comprising a Kabat CDR-H1 sequence comprising, consisting of, or consisting essentially of SEQ ID NO: 70. In some aspects, the antibody comprises a V_{H} sequence comprising a Kabat CDR-H1 sequence comprising, consisting of, or consisting essentially of SEQ ID NO: 71. In some aspects, the antibody comprises a V_{H} sequence comprising a Kabat CDR-H1 sequence comprising, consisting of, or consisting essentially of SEQ ID NO: 72. In some aspects, the antibody comprises a V_{H} sequence comprising a Kabat CDR-H1 sequence comprising, consisting of, or consisting essentially of SEQ ID NO: 73. In some aspects, the antibody comprises a V_{H} sequence comprising a Kabat CDR-H1 sequence comprising, consisting of, or consisting essentially of SEQ ID NO: 74. In some aspects, the antibody comprises a V_{H} sequence comprising a Kabat CDR-H1 sequence comprising, consisting of, or consisting essentially of SEQ ID NO: 75. In some aspects, the antibody comprises a V_{H} sequence comprising a Kabat CDR-H1 sequence comprising, consisting of, or consisting essentially of SEQ ID NO: 76. In some aspects, the antibody comprises a V_{H} sequence comprising a Kabat CDR-H1 sequence comprising, consisting of, or consisting essentially of SEQ ID NO: 77. In some aspects, the antibody comprises a V_{H} sequence comprising a Kabat CDR-H1 sequence comprising, consisting of, or consisting essentially of SEQ ID NO: 78. In some aspects, the antibody comprises a V_{H} sequence comprising a Kabat CDR-H1 sequence comprising, consisting of, or consisting essentially of SEQ ID NO: 79. In some aspects, the antibody comprises a V_{H} sequence comprising a Kabat CDR-H1 sequence comprising, consisting of, or consisting essentially of SEQ ID NO: 80. In some aspects, the antibody comprises a V_{H} sequence comprising a Kabat CDR-H1 sequence comprising, consisting of, or consisting essentially of SEQ ID NO: 81. In some aspects, the antibody comprises a V_{H} sequence comprising a Kabat CDR-H1 sequence comprising, consisting of, or consisting essentially of SEQ ID NO: 82. In some aspects, the antibody comprises a V_{H} sequence comprising a Kabat CDR-H1 sequence comprising, consisting of, or consisting essentially of SEQ ID NO: 83. In some aspects, the antibody comprises a V_{H} sequence comprising a Kabat CDR-H1 sequence comprising, consisting of, or consisting essentially of SEQ ID NO: 84. In some aspects, the antibody comprises a V_{H} sequence comprising a Kabat CDR-H1 sequence comprising, consisting of, or consisting essentially of SEQ ID NO: 85. In some aspects, the antibody comprises a V_{H} sequence comprising a Kabat CDR-H1 sequence comprising, consisting of, or consisting essentially of SEQ ID NO: 86. In some aspects, the antibody comprises a V_{H} sequence comprising a Kabat CDR-H1 sequence comprising, consisting of, or consisting essentially of SEQ ID NO: 87. In some aspects, the antibody comprises a V_{H} sequence comprising a Kabat CDR-H1 sequence comprising, consisting of, or consisting essentially of SEQ ID NO: 88. In some aspects, the antibody comprises a V_{H} sequence comprising a Kabat CDR-H1 sequence comprising, consisting of, or consisting essentially of SEQ ID NO: 89. In some aspects, the antibody comprises a V_{H} sequence comprising a Kabat CDR-H1 sequence comprising, consisting of, or consisting essentially of SEQ ID NO: 90. In some aspects, the antibody comprises a V_{H} sequence comprising a Kabat CDR-H1 sequence comprising, consisting of, or consisting essentially of SEQ ID NO: 91. In some aspects, the antibody comprises a V_{H} sequence comprising a Kabat CDR-H1 sequence comprising, consisting of, or consisting essentially of SEQ ID NO: 92. In some aspects, the antibody comprises a V_{H} sequence comprising a Kabat CDR-H1 sequence comprising, consisting of, or consisting essentially of SEQ ID NO: 93. In some aspects, the antibody comprises a V_{H} sequence comprising a Kabat CDR-H1 sequence comprising, consisting of, or consisting essentially of SEQ ID NO: 94. In some aspects, the antibody comprises a V_{H} sequence comprising a Kabat CDR-H1 sequence comprising, consisting of, or consisting essentially of SEQ ID NO: 95. In some aspects, the antibody comprises a V_{H} sequence comprising a Kabat CDR-H1 sequence comprising, consisting of, or consisting essentially of SEQ ID NO: 96. In some aspects, the antibody comprises a V_{H} sequence comprising a Kabat CDR-H1 sequence comprising, consisting of, or consisting essentially of SEQ ID NO: 97. In some aspects, the antibody comprises a V_{H} sequence comprising a Kabat CDR-H1 sequence comprising, consisting of, or consisting essentially of SEQ ID NO: 98. In some aspects, the antibody comprises a V_{H} sequence comprising a Kabat CDR-H1 sequence comprising, consisting of, or consisting essentially of SEQ ID NO: 99. In some aspects, the antibody comprises a V_{H} sequence comprising a Kabat CDR-H1 sequence comprising, consisting of, or consisting essentially of SEQ ID NO: 100. In some aspects, the antibody comprises a V_{H} sequence comprising a Kabat CDR-H1 sequence comprising, consisting of, or consisting essentially of SEQ ID NO: 101. In some aspects, the antibody comprises a V_{H} sequence comprising a Kabat CDR-H1 sequence comprising, consisting of, or consisting essentially of SEQ ID NO: 102. In some aspects, the antibody comprises a V_{H} sequence comprising a Kabat CDR-H1 sequence comprising, consisting of, or consisting essentially of SEQ ID NO: 103. In some aspects, the antibody comprises a V_{H} sequence comprising a Kabat CDR-H1 sequence comprising, consisting of, or consisting essentially of SEQ ID NO: 104. In some aspects, the antibody comprises a V_{H} sequence comprising a Kabat CDR-H1 sequence comprising, consisting of, or consisting essentially of SEQ ID NO: 105. In some aspects, the antibody comprises a V_{H} sequence comprising a Kabat CDR-H1 sequence comprising, consisting of, or consisting essentially of SEQ ID NO: 106. In some aspects, the antibody comprises a V_{H} sequence comprising a Kabat CDR-H1 sequence comprising, consisting of, or consisting essentially of SEQ ID NO: 107. In some aspects, the antibody comprises a V_{H} sequence comprising a Kabat CDR-H1 sequence comprising, consisting of, or consisting essentially of SEQ ID NO: 108. In some aspects, the antibody comprises a V_{H} sequence comprising a Kabat CDR-H1 sequence comprising, consisting of, or consisting essentially of SEQ ID NO: 109. In some aspects, the antibody comprises a V_{H} sequence comprising a Kabat CDR-H1 sequence comprising, consisting of, or consisting essentially of SEQ ID NO: 110. In some aspects, the antibody comprises a V_{H} sequence comprising a Kabat CDR-H1 sequence comprising, consisting of, or consisting essentially of SEQ ID NO: 111. In some aspects, the antibody comprises a V_{H} sequence comprising a Kabat CDR-H1 sequence comprising, consisting of, or consisting essentially of SEQ ID NO: 112. In some aspects, the antibody comprises a V_{H} sequence comprising a Kabat CDR-H1 sequence comprising, consisting of, or consisting essentially of SEQ ID NO: 113. In some aspects, the antibody comprises a V_{H} sequence comprising a Kabat CDR-H1 sequence comprising, consisting of, or consisting essentially of SEQ ID NO: 114. In some aspects, the antibody comprises a V_{H} sequence comprising a Kabat CDR-H1 sequence comprising, consisting of, or consisting essentially of SEQ ID NO: 115. In some aspects, the antibody comprises a V_{H} sequence comprising a Kabat CDR-H1 sequence comprising, consisting of, or consisting essentially of SEQ ID NO: 116. In some aspects, the antibody comprises a V_{H} sequence comprising a Kabat CDR-H1 sequence comprising, consisting of, or consisting essentially of SEQ ID NO: 117. In some aspects, the antibody comprises a V_{H} sequence comprising a Kabat CDR-H1 sequence comprising, consisting of, or consisting essentially of SEQ ID NO: 118. In some aspects, the antibody comprises a V_{H} sequence comprising a Kabat CDR-H1 sequence comprising, consisting of, or consisting essentially of SEQ ID NO: 119. In some aspects, the antibody comprises a V_{H} sequence comprising a Kabat CDR-H1 sequence comprising, consisting of, or consisting essentially of SEQ ID NO: 120. In some aspects, the antibody comprises a V_{H} sequence comprising a Kabat CDR-H1 sequence comprising, consisting of, or consisting essentially of SEQ ID NO: 121.

2.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H2 sequence comprising, consisting of, or consisting essentially of a sequence selected from SEQ ID NOs: 181-239. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 308-366.

2.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 240-298, and a Kabat CDR-H1 sequence comprising, consisting of, or consisting essentially of a sequence selected from SEQ ID NOs: 63-121. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 308-366.

2.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 63-121 and a Kabat CDR-H2 sequence comprising, consisting of, or consisting essentially of a sequence selected from SEQ ID NOs: 181-239. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 308-366.

2.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 63-121, a Kabat CDR-H2 sequence comprising, consisting of, or consisting essentially of a sequence selected from SEQ ID NOs: 181-239, and a Kabat CDR-H3 sequence comprising, consisting of, or consisting essentially of a sequence selected from SEQ ID NOs: 240-298. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 308-366.

2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

2.2.2.1. Chotia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 308-366.

In some embodiments, the antibody comprises a V_{H} sequence comprising a Chothia CDR-H3 sequence comprising, consisting of, or consisting essentially of a sequence selected from SEQ ID NOs: 240-298. In some aspects, the antibody comprises a V_{H} sequence comprising a Chothia CDR-H3 sequence comprising, consisting of, or consisting essentially of SEQ ID NO: 240. In some aspects, the antibody comprises a V_{H} sequence comprising a Chothia CDR-H3 sequence comprising, consisting of, or consisting essentially of SEQ ID NO: 241. In some aspects, the antibody comprises a V_{H} sequence comprising a Chothia CDR-H3 sequence comprising, consisting of, or consisting essentially of SEQ ID NO: 242. In some aspects, the antibody comprises a V_{H} sequence comprising a Chothia CDR-H3 sequence comprising, consisting of, or consisting essentially of SEQ ID NO: 243. In some aspects, the antibody comprises a V_{H} sequence comprising a Chothia CDR-H3 sequence comprising, consisting of, or consisting essentially of SEQ ID NO: 244. In some aspects, the antibody comprises a V_{H} sequence comprising a Chothia CDR-H3 sequence comprising, consisting of, or consisting essentially of SEQ ID NO: 245. In some aspects, the antibody comprises a V_{H} sequence comprising a Chothia CDR-H3 sequence comprising, consisting of, or consisting essentially of SEQ ID NO: 246. In some aspects, the antibody comprises a V_{H} sequence comprising a Chothia CDR-H3 sequence comprising, consisting of, or consisting essentially of SEQ ID NO: 247. In some aspects, the antibody comprises a V_{H} sequence comprising a Chothia CDR-H3 sequence comprising, consisting of, or consisting essentially of SEQ ID NO: 248. In some aspects, the antibody comprises a V_{H} sequence comprising a Chothia CDR-H3 sequence comprising, consisting of, or consisting essentially of SEQ ID NO: 249. In some aspects, the antibody comprises a V_{H} sequence comprising a Chothia CDR-H3 sequence comprising, consisting of, or consisting essentially of SEQ ID NO: 250. In some aspects, the antibody comprises a V_{H} sequence comprising a Chothia CDR-H3 sequence comprising, consisting of, or consisting essentially of SEQ ID NO: 251. In some aspects, the antibody comprises a V_{H} sequence comprising a Chothia CDR-H3 sequence comprising, consisting of, or consisting essentially of SEQ ID NO: 252. In some aspects, the antibody comprises a V_{H} sequence comprising a Chothia CDR-H3 sequence comprising, consisting of, or consisting essentially of SEQ ID NO: 253. In some aspects, the antibody comprises a V_{H} sequence comprising a Chothia CDR-H3 sequence comprising, consisting of, or consisting essentially of SEQ ID NO: 254. In some aspects, the antibody comprises a V_{H} sequence comprising a Chothia CDR-H3 sequence comprising, consisting of, or consisting essentially of SEQ ID NO: 255. In some aspects, the antibody comprises a V_{H} sequence comprising a Chothia CDR-H3 sequence comprising, consisting of, or consisting essentially of SEQ ID NO: 256. In some aspects, the antibody comprises a V_{H} sequence comprising a Chothia CDR-H3 sequence comprising, consisting of, or consisting essentially of SEQ ID NO: 257. In some aspects, the antibody comprises a V_{H} sequence comprising a Chothia CDR-H3 sequence comprising, consisting of, or consisting essentially of SEQ ID NO: 258. In some aspects, the antibody comprises a V_{H} sequence comprising a Chothia CDR-H3 sequence comprising, consisting of, or consisting essentially of SEQ ID NO: 259. In some aspects, the antibody comprises a V_{H} sequence comprising a Chothia CDR-H3 sequence comprising, consisting of, or consisting essentially of SEQ ID NO: 260. In some aspects, the antibody comprises a V_{H} sequence comprising a Chothia CDR-H3 sequence comprising, consisting of, or consisting essentially of SEQ ID NO: 261. In some aspects, the antibody comprises a V_{H} sequence comprising a Chothia CDR-H3 sequence comprising, consisting of, or consisting essentially of SEQ ID NO: 262. In some aspects, the antibody comprises a V_{H} sequence comprising a Chothia CDR-H3 sequence comprising, consisting of, or consisting essentially of SEQ ID NO: 263. In some aspects, the antibody comprises a V_{H} sequence comprising a Chothia CDR-H3 sequence comprising, consisting of, or consisting essentially of SEQ ID NO: 264. In some aspects, the antibody comprises a V_{H} sequence comprising a Chothia CDR-H3 sequence comprising, consisting of, or consisting essentially of SEQ ID NO: 265. In some aspects, the antibody comprises a V_{H} sequence comprising a Chothia CDR-H3 sequence comprising, consisting of, or consisting essentially of SEQ ID NO: 266. In some aspects, the antibody comprises a V_{H} sequence comprising a Chothia CDR-H3 sequence comprising, consisting of, or consisting essentially of SEQ ID NO: 267. In some aspects, the antibody comprises a V_{H} sequence comprising a Chothia CDR-H3 sequence comprising, consisting of, or consisting essentially of SEQ ID NO: 268. In some aspects, the antibody comprises a V_{H} sequence comprising a Chothia CDR-H3 sequence comprising, consisting of, or consisting essentially of SEQ ID NO: 269. In some aspects, the antibody comprises a V_{H} sequence comprising a Chothia CDR-H3 sequence comprising, consisting of, or consisting essentially of SEQ ID NO: 270. In some aspects, the antibody comprises a V_{H} sequence comprising a Chothia CDR-H3 sequence comprising, consisting of, or consisting essentially of SEQ ID NO: 271. In some aspects, the antibody comprises a V_{H} sequence comprising a Chothia CDR-H3 sequence comprising, consisting of, or consisting essentially of SEQ ID NO: 272. In some aspects, the antibody comprises a V_{H} sequence comprising a Chothia CDR-H3 sequence comprising, consisting of, or consisting essentially of SEQ ID NO: 273. In some aspects, the antibody comprises a V_{H} sequence comprising a Chothia CDR-H3 sequence comprising, consisting of, or consisting essentially of SEQ ID NO: 274. In some aspects, the antibody comprises a V_{H} sequence comprising a Chothia CDR-H3 sequence comprising, consisting of, or consisting essentially of SEQ ID NO: 275. In some aspects, the antibody comprises a V_{H} sequence comprising a Chothia CDR-H3 sequence comprising, consisting of, or consisting essentially of SEQ ID NO: 276. In some aspects, the antibody comprises a V_{H} sequence comprising a Chothia CDR-H3 sequence comprising, consisting of, or consisting essentially of SEQ ID NO: 277. In some aspects, the antibody comprises a V_{H} sequence comprising a Chothia CDR-H3 sequence comprising, consisting of, or consisting essentially of SEQ ID NO: 278. In some aspects, the antibody comprises a V_{H} sequence comprising a Chothia CDR-H3 sequence comprising, consisting of, or consisting essentially of SEQ ID NO: 279. In some aspects, the antibody comprises a V_{H} sequence comprising a Chothia CDR-H3 sequence comprising, consisting of, or consisting essentially of SEQ ID NO: 280. In some aspects, the antibody comprises a V_{H} sequence comprising a Chothia CDR-H3 sequence comprising, consisting of, or consisting essentially of SEQ ID NO: 281. In some aspects, the antibody comprises a V_{H} sequence comprising a Chothia CDR-H3 sequence comprising, consisting of, or consisting essentially of SEQ ID NO: 282. In some aspects, the antibody comprises a V_{H} sequence comprising a Chothia CDR-H3 sequence comprising, consisting of, or consisting essentially of SEQ ID NO: 283. In some aspects, the antibody comprises a V_{H} sequence comprising a Chothia CDR-H3 sequence comprising, consisting of, or consisting essentially of SEQ ID NO: 284. In some aspects, the antibody comprises a V_{H} sequence comprising a Chothia CDR-H3 sequence comprising, consisting of, or consisting essentially of SEQ ID NO: 285. In some aspects, the antibody comprises a V_{H} sequence comprising a Chothia CDR-H3 sequence comprising, consisting of, or consisting essentially of SEQ ID NO: 286. In some aspects, the antibody comprises a V_{H} sequence comprising a Chothia CDR-H3 sequence comprising, consisting of, or consisting essentially of SEQ ID NO: 287. In some aspects, the antibody comprises a V_{H} sequence comprising a Chothia CDR-H3 sequence comprising, consisting of, or consisting essentially of SEQ ID NO: 288. In some aspects, the antibody comprises a V_{H} sequence comprising a Chothia CDR-H3 sequence comprising, consisting of, or consisting essentially of SEQ ID NO: 289. In some aspects, the antibody comprises a V_{H} sequence comprising a Chothia CDR-H3 sequence comprising, consisting of, or consisting essentially of SEQ ID NO: 290. In some aspects, the antibody comprises a V_{H} sequence comprising a Chothia CDR-H3 sequence comprising, consisting of, or consisting essentially of SEQ ID NO: 291. In some aspects, the antibody comprises a V_{H} sequence comprising a Chothia CDR-H3 sequence comprising, consisting of, or consisting essentially of SEQ ID NO: 292. In some aspects, the antibody comprises a V_{H} sequence comprising a Chothia CDR-H3 sequence comprising, consisting of, or consisting essentially of SEQ ID NO: 293. In some aspects, the antibody comprises a V_{H} sequence comprising a Chothia CDR-H3 sequence comprising, consisting of, or consisting essentially of SEQ ID NO: 294. In some aspects, the antibody comprises a V_{H} sequence comprising a Chothia CDR-H3 sequence comprising, consisting of, or consisting essentially of SEQ ID NO: 295. In some aspects, the antibody comprises a V_{H} sequence comprising a Chothia CDR-H3 sequence comprising, consisting of, or consisting essentially of SEQ ID NO: 296. In some aspects, the antibody comprises a V_{H} sequence comprising a Chothia CDR-H3 sequence comprising, consisting of, or consisting essentially of SEQ ID NO: 297. In some aspects, the antibody comprises a V_{H} sequence comprising a Chothia CDR-H3 sequence comprising, consisting of, or consisting essentially of SEQ ID NO: 298.

2.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 308-366.

In some embodiments, the antibody comprises a V_{H} sequence comprising a Chothia CDR-H2 sequence comprising, consisting of, or consisting essentially of a sequence selected from SEQ ID NOs: 122-180. In some aspects, the antibody comprises a V_{H} sequence comprising a Chothia CDR-H2 sequence comprising, consisting of, or consisting essentially of SEQ ID NO: 122. In some aspects, the antibody comprises a V_{H} sequence comprising a Chothia CDR-H2 sequence comprising, consisting of, or consisting essentially of SEQ ID NO: 123. In some aspects, the antibody comprises a V_{H} sequence comprising a Chothia CDR-H2 sequence comprising, consisting of, or consisting essentially of SEQ ID NO: 124. In some aspects, the antibody comprises a V_{H} sequence comprising a Chothia CDR-H2 sequence comprising, consisting of, or consisting essentially of SEQ ID NO: 125. In some aspects, the antibody comprises a V_{H} sequence comprising a Chothia CDR-H2 sequence comprising, consisting of, or consisting essentially of SEQ ID NO: 126. In some aspects, the antibody comprises a V_{H} sequence comprising a Chothia CDR-H2 sequence comprising, consisting of, or consisting essentially of SEQ ID NO: 127. In some aspects, the antibody comprises a V_{H} sequence comprising a Chothia CDR-H2 sequence comprising, consisting of, or consisting essentially of SEQ ID NO: 128. In some aspects, the antibody comprises a V_{H} sequence comprising a Chothia CDR-H2 sequence comprising, consisting of, or consisting essentially of SEQ ID NO: 129. In some aspects, the antibody comprises a V_{H} sequence comprising a Chothia CDR-H2 sequence comprising, consisting of, or consisting essentially of SEQ ID NO: 130. In some aspects, the antibody comprises a V_{H} sequence comprising a Chothia CDR-H2 sequence comprising, consisting of, or consisting essentially of SEQ ID NO: 131. In some aspects, the antibody comprises a V_{H} sequence comprising a Chothia CDR-H2 sequence comprising, consisting of, or consisting essentially of SEQ ID NO: 132. In some aspects, the antibody comprises a V_{H} sequence comprising a Chothia CDR-H2 sequence comprising, consisting of, or consisting essentially of SEQ ID NO: 133. In some aspects, the antibody comprises a V_{H} sequence comprising a Chothia CDR-H2 sequence comprising, consisting of, or consisting essentially of SEQ ID NO: 134. In some aspects, the antibody comprises a V_{H} sequence comprising a Chothia CDR-H2 sequence comprising, consisting of, or consisting essentially of SEQ ID NO: 135. In some aspects, the antibody comprises a V_{H} sequence comprising a Chothia CDR-H2 sequence comprising, consisting of, or consisting essentially of SEQ ID NO: 136. In some aspects, the antibody comprises a V_{H} sequence comprising a Chothia CDR-H2 sequence comprising, consisting of, or consisting essentially of SEQ ID NO: 137. In some aspects, the antibody comprises a V_{H} sequence comprising a Chothia CDR-H2 sequence comprising, consisting of, or consisting essentially of SEQ ID NO: 138. In some aspects, the antibody comprises a V_{H} sequence comprising a Chothia CDR-H2 sequence comprising, consisting of, or consisting essentially of SEQ ID NO: 139. In some aspects, the antibody comprises a V_{H} sequence comprising a Chothia CDR-H2 sequence comprising, consisting of, or consisting essentially of SEQ ID NO: 140. In some aspects, the antibody comprises a V_{H} sequence comprising a Chothia CDR-H2 sequence comprising, consisting of, or consisting essentially of SEQ ID NO: 141. In some aspects, the antibody comprises a V_{H} sequence comprising a Chothia CDR-H2 sequence comprising, consisting of, or consisting essentially of SEQ ID NO: 142. In some aspects, the antibody comprises a V_{H} sequence comprising a Chothia CDR-H2 sequence comprising, consisting of, or consisting essentially of SEQ ID NO: 143. In some aspects, the antibody comprises a V_{H} sequence comprising a Chothia CDR-H2 sequence comprising, consisting of, or consisting essentially of SEQ ID NO: 144. In some aspects, the antibody comprises a V_{H} sequence comprising a Chothia CDR-H2 sequence comprising, consisting of, or consisting essentially of SEQ ID NO: 145. In some aspects, the antibody comprises a V_{H} sequence comprising a Chothia CDR-H2 sequence comprising, consisting of, or consisting essentially of SEQ ID NO: 146. In some aspects, the antibody comprises a V_{H} sequence comprising a Chothia CDR-H2 sequence comprising, consisting of, or consisting essentially of SEQ ID NO: 147. In some aspects, the antibody comprises a V_{H} sequence comprising a Chothia CDR-H2 sequence comprising, consisting of, or consisting essentially of SEQ ID NO: 148. In some aspects, the antibody comprises a V_{H} sequence comprising a Chothia CDR-H2 sequence comprising, consisting of, or consisting essentially of SEQ ID NO: 149. In some aspects, the antibody comprises a V_{H} sequence comprising a Chothia CDR-H2 sequence comprising, consisting of, or consisting essentially of SEQ ID NO: 150. In some aspects, the antibody comprises a V_{H} sequence comprising a Chothia CDR-H2 sequence comprising, consisting of, or consisting essentially of SEQ ID NO: 151. In some aspects, the antibody comprises a V_{H} sequence comprising a Chothia CDR-H2 sequence comprising, consisting of, or consisting essentially of SEQ ID NO: 152. In some aspects, the antibody comprises a V_{H} sequence comprising a Chothia CDR-H2 sequence comprising, consisting of, or consisting essentially of SEQ ID NO: 153. In some aspects, the antibody comprises a V_{H} sequence comprising a Chothia CDR-H2 sequence comprising, consisting of, or consisting essentially of SEQ ID NO: 154. In some aspects, the antibody comprises a V_{H} sequence comprising a Chothia CDR-H2 sequence comprising, consisting of, or consisting essentially of SEQ ID NO: 155. In some aspects, the antibody comprises a V_{H} sequence comprising a Chothia CDR-H2 sequence comprising, consisting of, or consisting essentially of SEQ ID NO: 156. In some aspects, the antibody comprises a V_{H} sequence comprising a Chothia CDR-H2 sequence comprising, consisting of, or consisting essentially of SEQ ID NO: 157. In some aspects, the antibody comprises a V_{H} sequence comprising a Chothia CDR-H2 sequence comprising, consisting of, or consisting essentially of SEQ ID NO: 158. In some aspects, the antibody comprises a V_{H} sequence comprising a Chothia CDR-H2 sequence comprising, consisting of, or consisting essentially of SEQ ID NO: 159. In some aspects, the antibody comprises a V_{H} sequence comprising a Chothia CDR-H2 sequence comprising, consisting of, or consisting essentially of SEQ ID NO: 160. In some aspects, the antibody comprises a V_{H} sequence comprising a Chothia CDR-H2 sequence comprising, consisting of, or consisting essentially of SEQ ID NO: 161. In some aspects, the antibody comprises a V_{H} sequence comprising a Chothia CDR-H2 sequence comprising, consisting of, or consisting essentially of SEQ ID NO: 162. In some aspects, the antibody comprises a V_{H} sequence comprising a Chothia CDR-H2 sequence comprising, consisting of, or consisting essentially of SEQ ID NO: 163. In some aspects, the antibody comprises a V_{H} sequence comprising a Chothia CDR-H2 sequence comprising, consisting of, or consisting essentially of SEQ ID NO: 164. In some aspects, the antibody comprises a V_{H} sequence comprising a Chothia CDR-H2 sequence comprising, consisting of, or consisting essentially of SEQ ID NO: 165. In some aspects, the antibody comprises a V_{H} sequence comprising a Chothia CDR-H2 sequence comprising, consisting of, or consisting essentially of SEQ ID NO: 166. In some aspects, the antibody comprises a V_{H} sequence comprising a Chothia CDR-H2 sequence comprising, consisting of, or consisting essentially of SEQ ID NO: 167. In some aspects, the antibody comprises a V_{H} sequence comprising a Chothia CDR-H2 sequence comprising, consisting of, or consisting essentially of SEQ ID NO: 168. In some aspects, the antibody comprises a V_{H} sequence comprising a Chothia CDR-H2 sequence comprising, consisting of, or consisting essentially of SEQ ID NO: 169. In some aspects, the antibody comprises a V_{H} sequence comprising a Chothia CDR-H2 sequence comprising, consisting of, or consisting essentially of SEQ ID NO: 170. In some aspects, the antibody comprises a V_{H} sequence comprising a Chothia CDR-H2 sequence comprising, consisting of, or consisting essentially of SEQ ID NO: 171. In some aspects, the antibody comprises a V_{H} sequence comprising a Chothia CDR-H2 sequence comprising, consisting of, or consisting essentially of SEQ ID NO: 172. In some aspects, the antibody comprises a V_{H} sequence comprising a Chothia CDR-H2 sequence comprising, consisting of, or consisting essentially of SEQ ID NO: 173. In some aspects, the antibody comprises a V_{H} sequence comprising a Chothia CDR-H2 sequence comprising, consisting of, or consisting essentially of SEQ ID NO: 174. In some aspects, the antibody comprises a V_{H} sequence comprising a Chothia CDR-H2 sequence comprising, consisting of, or consisting essentially of SEQ ID NO: 175. In some aspects, the antibody comprises a V_{H} sequence comprising a Chothia CDR-H2 sequence comprising, consisting of, or consisting essentially of SEQ ID NO: 176. In some aspects, the antibody comprises a V_{H} sequence comprising a Chothia CDR-H2 sequence comprising, consisting of, or consisting essentially of SEQ ID NO: 177. In some aspects, the antibody comprises a V_{H} sequence comprising a Chothia CDR-H2 sequence comprising, consisting of, or consisting essentially of SEQ ID NO: 178. In some aspects, the antibody comprises a V_{H} sequence comprising a Chothia CDR-H2 sequence comprising, consisting of, or consisting essentially of SEQ ID NO: 179. In some aspects, the antibody comprises a V_{H} sequence comprising a Chothia CDR-H2 sequence comprising, consisting of, or consisting essentially of SEQ ID NO: 180.

2.2.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 308-366.

In some embodiments, the antibody comprises a V_{H} sequence comprising a Chothia CDR-H1 sequence comprising, consisting of, or consisting essentially of a sequence selected from SEQ ID NOs: 4-62. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a V_{H} sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14. In some aspects, the antibody comprises a V_{H} sequence comprising a Chothia CDR-H1 sequence comprising, consisting of, or consisting essentially of SEQ ID NO: 15. In some aspects, the antibody comprises a V_{H} sequence comprising a Chothia CDR-H1 sequence comprising, consisting of, or consisting essentially of SEQ ID NO: 16. In some aspects, the antibody comprises a V_{H} sequence comprising a Chothia CDR-H1 sequence comprising, consisting of, or consisting essentially of SEQ ID NO: 17. In some aspects, the antibody comprises a V_{H} sequence comprising a Chothia CDR-H1 sequence comprising, consisting of, or consisting essentially of SEQ ID NO: 18. In some aspects, the antibody comprises a V_{H} sequence comprising a Chothia CDR-H1 sequence comprising, consisting of, or consisting essentially of SEQ ID NO: 19. In some aspects, the antibody comprises a V_{H} sequence comprising a Chothia CDR-H1 sequence comprising, consisting of, or consisting essentially of SEQ ID NO: 20. In some aspects, the antibody comprises a V_{H} sequence comprising a Chothia CDR-H1 sequence comprising, consisting of, or consisting essentially of SEQ ID NO: 21. In some aspects, the antibody comprises a V_{H} sequence comprising a Chothia CDR-H1 sequence comprising, consisting of, or consisting essentially of SEQ ID NO: 22. In some aspects, the antibody comprises a V_{H} sequence comprising a Chothia CDR-H1 sequence comprising, consisting of, or consisting essentially of SEQ ID NO: 23. In some aspects, the antibody comprises a V_{H} sequence comprising a Chothia CDR-H1 sequence comprising, consisting of, or consisting essentially of SEQ ID NO: 24. In some aspects, the antibody comprises a V_{H} sequence comprising a Chothia CDR-H1 sequence comprising, consisting of, or consisting essentially of SEQ ID NO: 25. In some aspects, the antibody comprises a V_{H} sequence comprising a Chothia CDR-H1 sequence comprising, consisting of, or consisting essentially of SEQ ID NO: 26. In some aspects, the antibody comprises a V_{H} sequence comprising a Chothia CDR-H1 sequence comprising, consisting of, or consisting essentially of SEQ ID NO: 27. In some aspects, the antibody comprises a V_{H} sequence comprising a Chothia CDR-H1 sequence comprising, consisting of, or consisting essentially of SEQ ID NO: 28. In some aspects, the antibody comprises a V_{H} sequence comprising a Chothia CDR-H1 sequence comprising, consisting of, or consisting essentially of SEQ ID NO: 29. In some aspects, the antibody comprises a V_{H} sequence comprising a Chothia CDR-H1 sequence comprising, consisting of, or consisting essentially of SEQ ID NO: 30. In some aspects, the antibody comprises a V_{H} sequence comprising a Chothia CDR-H1 sequence comprising, consisting of, or consisting essentially of SEQ ID NO: 31. In some aspects, the antibody comprises a V_{H} sequence comprising a Chothia CDR-H1 sequence comprising, consisting of, or consisting essentially of SEQ ID NO: 32. In some aspects, the antibody comprises a V_{H} sequence comprising a Chothia CDR-H1 sequence comprising, consisting of, or consisting essentially of SEQ ID NO: 33. In some aspects, the antibody comprises a V_{H} sequence comprising a Chothia CDR-H1 sequence comprising, consisting of, or consisting essentially of SEQ ID NO: 34. In some aspects, the antibody comprises a V_{H} sequence comprising a Chothia CDR-H1 sequence comprising, consisting of, or consisting essentially of SEQ ID NO: 35. In some aspects, the antibody comprises a V_{H} sequence comprising a Chothia CDR-H1 sequence comprising, consisting of, or consisting essentially of SEQ ID NO: 36. In some aspects, the antibody comprises a V_{H} sequence comprising a Chothia CDR-H1 sequence comprising, consisting of, or consisting essentially of SEQ ID NO: 37. In some aspects, the antibody comprises a V_{H} sequence comprising a Chothia CDR-H1 sequence comprising, consisting of, or consisting essentially of SEQ ID NO: 38. In some aspects, the antibody comprises a V_{H} sequence comprising a Chothia CDR-H1 sequence comprising, consisting of, or consisting essentially of SEQ ID NO: 39. In some aspects, the antibody comprises a V_{H} sequence comprising a Chothia CDR-H1 sequence comprising, consisting of, or consisting essentially of SEQ ID NO: 40. In some aspects, the antibody comprises a V_{H} sequence comprising a Chothia CDR-H1 sequence comprising, consisting of, or consisting essentially of SEQ ID NO: 41. In some aspects, the antibody comprises a V_{H} sequence comprising a Chothia CDR-H1 sequence comprising, consisting of, or consisting essentially of SEQ ID NO: 42. In some aspects, the antibody comprises a V_{H} sequence comprising a Chothia CDR-H1 sequence comprising, consisting of, or consisting essentially of SEQ ID NO: 43. In some aspects, the antibody comprises a V_{H} sequence comprising a Chothia CDR-H1 sequence comprising, consisting of, or consisting essentially of SEQ ID NO: 44. In some aspects, the antibody comprises a V_{H} sequence comprising a Chothia CDR-H1 sequence comprising, consisting of, or consisting essentially of SEQ ID NO: 45. In some aspects, the antibody comprises a V_{H} sequence comprising a Chothia CDR-H1 sequence comprising, consisting of, or consisting essentially of SEQ ID NO: 46. In some aspects, the antibody comprises a V_{H} sequence comprising a Chothia CDR-H1 sequence comprising, consisting of, or consisting essentially of SEQ ID NO: 47. In some aspects, the antibody comprises a V_{H} sequence comprising a Chothia CDR-H1 sequence comprising, consisting of, or consisting essentially of SEQ ID NO: 48. In some aspects, the antibody comprises a V_{H} sequence comprising a Chothia CDR-H1 sequence comprising, consisting of, or consisting essentially of SEQ ID NO: 49. In some aspects, the antibody comprises a V_{H} sequence comprising a Chothia CDR-H1 sequence comprising, consisting of, or consisting essentially of SEQ ID NO: 50. In some aspects, the antibody comprises a V_{H} sequence comprising a Chothia CDR-H1 sequence comprising, consisting of, or consisting essentially of SEQ ID NO: 51. In some aspects, the antibody comprises a V_{H} sequence comprising a Chothia CDR-H1 sequence comprising, consisting of, or consisting essentially of SEQ ID NO: 52. In some aspects, the antibody comprises a V_{H} sequence comprising a Chothia CDR-H1 sequence comprising, consisting of, or consisting essentially of SEQ ID NO: 53. In some aspects, the antibody comprises a V_{H} sequence comprising a Chothia CDR-H1 sequence comprising, consisting of, or consisting essentially of SEQ ID NO: 54. In some aspects, the antibody comprises a V_{H} sequence comprising a Chothia CDR-H1 sequence comprising, consisting of, or consisting essentially of SEQ ID NO: 55. In some aspects, the antibody comprises a V_{H} sequence comprising a Chothia CDR-H1 sequence comprising, consisting of, or consisting essentially of SEQ ID NO: 56. In some aspects, the antibody comprises a V_{H} sequence comprising a Chothia CDR-H1 sequence comprising, consisting of, or consisting essentially of SEQ ID NO: 57. In some aspects, the antibody comprises a V_{H} sequence comprising a Chothia CDR-H1 sequence comprising, consisting of, or consisting essentially of SEQ ID NO: 58. In some aspects, the antibody comprises a V_{H} sequence comprising a Chothia CDR-H1 sequence comprising, consisting of, or consisting essentially of SEQ ID NO: 59. In some aspects, the antibody comprises a V_{H} sequence comprising a Chothia CDR-H1 sequence comprising, consisting of, or consisting essentially of SEQ ID NO: 60. In some aspects, the antibody comprises a V_{H} sequence comprising a Chothia CDR-H1 sequence comprising, consisting of, or consisting essentially of SEQ ID NO: 61. In some aspects, the antibody comprises a V_{H} sequence comprising a Chothia CDR-H1 sequence comprising, consisting of, or consisting essentially of SEQ ID NO: 62.

2.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H2 sequence comprising, consisting of, or consisting essentially of a sequence selected from SEQ ID NOs: 122-180. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 308-366.

2.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 240-298, and a Chothia CDR-H1 sequence comprising, consisting of, or consisting essentially of a sequence selected from SEQ ID NOs: 4-62. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 308-366.

2.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 4-62 and a Chothia CDR-H2 sequence comprising, consisting of, or consisting essentially of a sequence selected from SEQ ID NOs: 122-180. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 308-366.

2.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 4-62, a Chothia CDR-H2 sequence comprising, consisting of, or consisting essentially of a sequence selected from SEQ ID NOs: 122-180, and a Chothia CDR-H3 sequence comprising, consisting of, or consisting essentially of a sequence selected from SEQ ID NOs: 240-298. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 308-366.

2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

2.3. V_{H} Sequences

In some embodiments, the antibody comprises, consists of, or consists essentially of a V_{H} sequence provided in SEQ ID NOs: 308-366.

In some embodiments, the antibody comprises a V_{H} sequence comprising, consisting of, or consisting essentially of a sequence selected from SEQ ID NOs: 308-366. In some aspects, the antibody comprises a V_{H} sequence comprising, consisting of, or consisting essentially of SEQ ID NO: 308. In some aspects, the antibody comprises a V_{H} sequence comprising, consisting of, or consisting essentially of SEQ ID NO: 309. In some aspects, the antibody comprises a V_{H} sequence comprising, consisting of, or consisting essentially of SEQ ID NO: 310. In some aspects, the antibody comprises a V_{H} sequence comprising, consisting of, or consisting essentially of SEQ ID NO: 311. In some aspects, the antibody comprises a V_{H} sequence comprising, consisting of, or consisting essentially of SEQ ID NO: 312. In some aspects, the antibody comprises a V_{H} sequence comprising, consisting of, or consisting essentially of SEQ ID NO: 313. In some aspects, the antibody comprises a V_{H} sequence comprising, consisting of, or consisting essentially of SEQ ID NO: 314. In some aspects, the antibody comprises a V_{H} sequence comprising, consisting of, or consisting essentially of SEQ ID NO: 315. In some aspects, the antibody comprises a V_{H} sequence comprising, consisting of, or consisting essentially of SEQ ID NO: 316. In some aspects, the antibody comprises a V_{H} sequence comprising, consisting of, or consisting essentially of SEQ ID NO: 317. In some aspects, the antibody comprises a V_{H} sequence comprising, consisting of, or consisting essentially of SEQ ID NO: 318. In some aspects, the antibody comprises a V_{H} sequence comprising, consisting of, or consisting essentially of SEQ ID NO: 319. In some aspects, the antibody comprises a V_{H} sequence comprising, consisting of, or consisting essentially of SEQ ID NO: 320. In some aspects, the antibody comprises a V_{H} sequence comprising, consisting of, or consisting essentially of SEQ ID NO: 321. In some aspects, the antibody comprises a V_{H} sequence comprising, consisting of, or consisting essentially of SEQ ID NO: 322. In some aspects, the antibody comprises a V_{H} sequence comprising, consisting of, or consisting essentially of SEQ ID NO: 323. In some aspects, the antibody comprises a V_{H} sequence comprising, consisting of, or consisting essentially of SEQ ID NO: 324. In some aspects, the antibody comprises a V_{H} sequence comprising, consisting of, or consisting essentially of SEQ ID NO: 325. In some aspects, the antibody comprises a V_{H} sequence comprising, consisting of, or consisting essentially of SEQ ID NO: 326. In some aspects, the antibody comprises a V_{H} sequence comprising, consisting of, or consisting essentially of SEQ ID NO: 327. In some aspects, the antibody comprises a V_{H} sequence comprising, consisting of, or consisting essentially of SEQ ID NO: 328. In some aspects, the antibody comprises a V_{H} sequence comprising, consisting of, or consisting essentially of SEQ ID NO: 329. In some aspects, the antibody comprises a V_{H} sequence comprising, consisting of, or consisting essentially of SEQ ID NO: 330. In some aspects, the antibody comprises a V_{H} sequence comprising, consisting of, or consisting essentially of SEQ ID NO: 331. In some aspects, the antibody comprises a V_{H} sequence comprising, consisting of, or consisting essentially of SEQ ID NO: 332. In some aspects, the antibody comprises a V_{H} sequence comprising, consisting of, or consisting essentially of SEQ ID NO: 333. In some aspects, the antibody comprises a V_{H} sequence comprising, consisting of, or consisting essentially of SEQ ID NO: 334. In some aspects, the antibody comprises a V_{H} sequence comprising, consisting of, or consisting essentially of SEQ ID NO: 335. In some aspects, the antibody comprises a V_{H} sequence comprising, consisting of, or consisting essentially of SEQ ID NO: 336. In some aspects, the antibody comprises a V_{H} sequence comprising, consisting of, or consisting essentially of SEQ ID NO: 337. In some aspects, the antibody comprises a V_{H} sequence comprising, consisting of, or consisting essentially of SEQ ID NO: 338. In some aspects, the antibody comprises a V_{H} sequence comprising, consisting of, or consisting essentially of SEQ ID NO: 339. In some aspects, the antibody comprises a V_{H} sequence comprising, consisting of, or consisting essentially of SEQ ID NO: 340. In some aspects, the antibody comprises a V_{H} sequence comprising, consisting of, or consisting essentially of SEQ ID NO: 341. In some aspects, the antibody comprises a V_{H} sequence comprising, consisting of, or consisting essentially of SEQ ID NO: 342. In some aspects, the antibody comprises a V_{H} sequence comprising, consisting of, or consisting essentially of SEQ ID NO: 343. In some aspects, the antibody comprises a V_{H} sequence comprising, consisting of, or consisting essentially of SEQ ID NO: 344. In some aspects, the antibody comprises a V_{H} sequence comprising, consisting of, or consisting essentially of SEQ ID NO: 345. In some aspects, the antibody comprises a V_{H} sequence comprising, consisting of, or consisting essentially of SEQ ID NO: 346. In some aspects, the antibody comprises a V_{H} sequence comprising, consisting of, or consisting essentially of SEQ ID NO: 347. In some aspects, the antibody comprises a V_{H} sequence comprising, consisting of, or consisting essentially of SEQ ID NO: 348. In some aspects, the antibody comprises a V_{H} sequence comprising, consisting of, or consisting essentially of SEQ ID NO: 349. In some aspects, the antibody comprises a V_{H} sequence comprising, consisting of, or consisting essentially of SEQ ID NO: 350. In some aspects, the antibody comprises a V_{H} sequence comprising, consisting of, or consisting essentially of SEQ ID NO: 351. In some aspects, the antibody comprises a V_{H} sequence comprising, consisting of, or consisting essentially of SEQ ID NO: 352. In some aspects, the antibody comprises a V_{H} sequence comprising, consisting of, or consisting essentially of SEQ ID NO: 353. In some aspects, the antibody comprises a V_{H} sequence comprising, consisting of, or consisting essentially of SEQ ID NO: 354. In some aspects, the antibody comprises a V_{H} sequence comprising, consisting of, or consisting essentially of SEQ ID NO: 355. In some aspects, the antibody comprises a V_{H} sequence comprising, consisting of, or consisting essentially of SEQ ID NO: 356. In some aspects, the antibody comprises a V_{H} sequence comprising, consisting of, or consisting essentially of SEQ ID NO: 357. In some aspects, the antibody comprises a V_{H} sequence comprising, consisting of, or consisting essentially of SEQ ID NO: 358. In some aspects, the antibody comprises a V_{H} sequence comprising, consisting of, or consisting essentially of SEQ ID NO: 359. In some aspects, the antibody comprises a V_{H} sequence comprising, consisting of, or consisting essentially of SEQ ID NO: 360. In some aspects, the antibody comprises a V_{H} sequence comprising, consisting of, or consisting essentially of SEQ ID NO: 361. In some aspects, the antibody comprises a V_{H} sequence comprising, consisting of, or consisting essentially of SEQ ID NO: 362. In some aspects, the antibody comprises a V_{H} sequence comprising, consisting of, or consisting essentially of SEQ ID NO: 363. In some aspects, the antibody comprises a V_{H} sequence comprising, consisting of, or consisting essentially of SEQ ID NO: 364. In some aspects, the antibody comprises a V_{H} sequence comprising, consisting of, or consisting essentially of SEQ ID NO: 365. In some aspects, the antibody comprises a V_{H} sequence comprising, consisting of, or consisting essentially of SEQ ID NO: 366.

2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.4. CDR-L3 Sequences

In some embodiments, the antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of a V_{L} sequence provided in SEQ ID NOs.: 367-369.

In some embodiments, the antibody comprises a CDR-L3 sequence comprising, consisting of, or consisting essentially of a sequence selected from SEQ ID NOs: 305-307. In some aspects, the antibody comprises a CDR-L3 sequence comprising, consisting of, or consisting essentially of SEQ ID NO: 305. In some aspects, the antibody comprises a CDR-L3 sequence comprising, consisting of, or consisting essentially of SEQ ID NO: 306. In some aspects, the antibody comprises a CDR-L3 sequence comprising, consisting of, or consisting essentially of SEQ ID NO: 307.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

2.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs.: 367-369.

In some embodiments, the antibody comprises a V_{L} sequence comprising a CDR-L3 sequence comprising, consisting of, or consisting essentially of a sequence selected from SEQ ID NOs: 305-307. In some aspects, the antibody comprises a V_{L} sequence comprising a CDR-L3 sequence comprising, consisting of, or consisting essentially of SEQ ID NO: 305. In some aspects, the antibody comprises a V_{L} sequence comprising a CDR-L3 sequence comprising, consisting of, or consisting essentially of SEQ ID NO: 306. In some aspects, the antibody comprises a V_{L} sequence comprising a CDR-L3 sequence comprising, consisting of, or consisting essentially of SEQ ID NO: 307.

2.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs.: 367-369.

In some embodiments, the antibody comprises a V_{L} sequence comprising a CDR-L2 sequence comprising, consisting of, or consisting essentially of a sequence selected from SEQ ID NOs: 302-304. In some aspects, the antibody comprises a V_{L} sequence comprising a CDR-L2 sequence comprising, consisting of, or consisting essentially of SEQ ID NO: 302. In some aspects, the antibody comprises a V_{L} sequence comprising a CDR-L2 sequence comprising, consisting of, or consisting essentially of SEQ ID NO: 303. In some aspects, the antibody comprises a V_{L} sequence comprising a CDR-L2 sequence comprising, consisting of, or consisting essentially of SEQ ID NO: 304.

2.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs.: 367-369.

In some embodiments, the antibody comprises a V_{L} sequence comprising a CDR-L1 sequence comprising, consisting of, or consisting essentially of a sequence selected from SEQ ID NOs: 299-301. In some aspects, the antibody comprises a V_{L} sequence comprising a CDR-L1 sequence comprising, consisting of, or consisting essentially of SEQ ID NO: 299. In some aspects, the antibody comprises a V_{L} sequence comprising a CDR-L1 sequence comprising, consisting of, or consisting essentially of SEQ ID NO: 300. In some aspects, the antibody comprises a V_{L} sequence comprising a CDR-L1 sequence comprising, consisting of, or consisting essentially of SEQ ID NO: 301.

2.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 305-307 and a CDR-L2 sequence comprising, consisting of, or consisting essentially of a sequence selected from SEQ ID NOs: 302-304. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs.: 367-369.

2.5.5. CDR-L3+CDR-L1

In some embodiments, the antibody comprises a V_{L} sequence comprising a CDR-L3 sequence comprising, consisting of, or consisting essentially of a sequence selected from SEQ ID NOs: 305-307 and a CDR-L1 sequence comprising, consisting of, or consisting essentially of a sequence selected from SEQ ID NOs: 299-301. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs.: 367-369.

2.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 299-301 and a CDR-L2 sequence comprising, consisting of, or consisting essentially of a sequence selected from SEQ ID NOs: 302-304. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs.: 367-369.

2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 299-301, a CDR-L2 sequence comprising, consisting of, or consisting essentially of a sequence selected from SEQ ID NOs: 302-304, and a CDR-L3 sequence comprising, consisting of, or consisting essentially of a sequence selected from SEQ ID NOs: 305-307. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs.: 367-369.

2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.6. V_{L} Sequences

In some embodiments, the antibody comprises, consists of, or consists essentially of a V_{L} sequence provided in SEQ ID NOs.: 367-369.

In some embodiments, the antibody comprises a V_{L} sequence comprising, consisting of, or consisting essentially of a sequence selected from SEQ ID NOs.: 367-369. In some aspects, the antibody comprises a V_{L} sequence comprising, consisting of, or consisting essentially of SEQ ID NO: 367. In some aspects, the antibody comprises a V_{L} sequence comprising, consisting of, or consisting essentially of SEQ ID NO: 368. In some aspects, the antibody comprises a V_{L} sequence comprising, consisting of, or consisting essentially of SEQ ID NO: 369.

2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.7. Pairs

2.7.1. CDR-H3-CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs: 240-298, and the CDR-L3 sequence is a CDR-L3 sequence comprising, consisting of, or consisting essentially of SEQ ID NOs: 305-307.

In some aspects, the CDR-H3-CDR-L3 pairs are selected from SEQ ID NO: 305 and SEQ ID NO: 240; SEQ ID NO: 305 and SEQ ID NO: 241; SEQ ID NO: 305 and SEQ ID NO: 242; SEQ ID NO: 305 and SEQ ID NO: 243; SEQ ID NO: 305 and SEQ ID NO: 244; SEQ ID NO: 305 and SEQ ID NO: 245; SEQ ID NO: 305 and SEQ ID NO: 246; SEQ ID NO: 305 and SEQ ID NO: 247; SEQ ID NO: 305 and SEQ ID NO: 248; SEQ ID NO: 305 and SEQ ID NO: 249; SEQ ID NO: 305 and SEQ ID NO: 250; SEQ ID NO: 305 and SEQ ID NO: 251; SEQ ID NO: 305 and SEQ ID NO: 252; SEQ ID NO: 305 and SEQ ID NO: 253; SEQ ID NO: 305 and SEQ ID NO: 254; SEQ ID NO: 305 and SEQ ID NO: 255; SEQ ID NO: 305 and SEQ ID NO: 256; SEQ ID NO: 305 and SEQ ID NO: 257; SEQ ID NO: 305 and SEQ ID NO: 258; SEQ ID NO: 305 and SEQ ID NO: 259; SEQ ID NO: 305 and SEQ ID NO: 260; SEQ ID NO: 305 and SEQ ID NO: 261; SEQ ID NO: 305 and SEQ ID NO: 262; SEQ ID NO: 305 and SEQ ID NO: 263; SEQ ID NO: 305 and SEQ ID NO: 264; SEQ ID NO: 305 and SEQ ID NO: 265; SEQ ID NO: 305 and SEQ ID NO: 266; SEQ ID NO: 305 and SEQ ID NO: 267; SEQ ID NO: 305 and SEQ ID NO: 268; SEQ ID NO: 305 and SEQ ID NO: 269; SEQ ID NO: 305 and SEQ ID NO: 270; SEQ ID NO: 305 and SEQ ID NO: 271; SEQ ID NO: 305 and SEQ ID NO: 272; SEQ ID NO: 305 and SEQ ID NO: 273; SEQ ID NO: 305 and SEQ ID NO: 274; SEQ ID NO: 305 and SEQ ID NO: 275; SEQ ID NO: 305 and SEQ ID NO: 276; SEQ ID NO: 305 and SEQ ID NO: 277; SEQ ID NO: 305 and SEQ ID NO: 278; SEQ ID NO: 305 and SEQ ID NO: 279; SEQ ID NO: 305 and SEQ ID NO: 280; SEQ ID NO: 305 and SEQ ID NO: 281; SEQ ID NO: 305 and SEQ ID NO: 282; SEQ ID NO: 305 and SEQ ID NO: 283; SEQ ID NO: 305 and SEQ ID NO: 284; SEQ ID NO: 305 and SEQ ID NO: 285; SEQ ID NO: 305 and SEQ ID NO: 286; SEQ ID NO: 305 and SEQ ID NO: 287; SEQ ID NO: 305 and SEQ ID NO: 288; SEQ ID NO: 305 and SEQ ID NO: 289; SEQ ID NO: 305 and SEQ ID NO: 290; SEQ ID NO: 305 and SEQ ID NO: 291; SEQ ID NO: 305 and SEQ ID NO: 292; SEQ ID NO: 305 and SEQ ID NO: 293; SEQ ID NO: 305 and SEQ ID NO: 294; SEQ ID NO: 305 and SEQ ID NO: 295; SEQ ID NO: 305 and SEQ ID NO: 296; SEQ ID NO: 305 and SEQ ID NO: 297; and SEQ ID NO: 305 and SEQ ID NO: 298.

In some aspects, the CDR-H3-CDR-L3 pairs are selected from SEQ ID NO: 306 and SEQ ID NO: 240; SEQ ID NO: 306 and SEQ ID NO: 241; SEQ ID NO: 306 and SEQ ID NO: 242; SEQ ID NO: 306 and SEQ ID NO: 243; SEQ ID NO: 306 and SEQ ID NO: 244; SEQ ID NO: 306 and SEQ ID NO: 245; SEQ ID NO: 306 and SEQ ID NO: 246; SEQ ID NO: 306 and SEQ ID NO: 247; SEQ ID NO: 306 and SEQ ID NO: 248; SEQ ID NO: 306 and SEQ ID NO: 249; SEQ ID NO: 306 and SEQ ID NO: 250; SEQ ID NO: 306 and SEQ ID NO: 251; SEQ ID NO: 306 and SEQ ID NO: 252; SEQ ID NO: 306 and SEQ ID NO: 253; SEQ ID NO: 306 and SEQ ID NO: 254; SEQ ID NO: 306 and SEQ ID NO: 255; SEQ ID NO: 306 and SEQ ID NO: 256; SEQ ID NO: 306 and SEQ ID NO: 257; SEQ ID NO: 306 and SEQ ID NO: 258; SEQ ID NO: 306 and SEQ ID NO: 259; SEQ ID NO: 306 and SEQ ID NO: 260; SEQ ID NO: 306 and SEQ ID NO: 261; SEQ ID NO: 306 and SEQ ID NO: 262; SEQ ID NO: 306 and SEQ ID NO: 263; SEQ ID NO: 306 and SEQ ID NO: 264; SEQ ID NO: 306 and SEQ ID NO: 265; SEQ ID NO: 306 and SEQ ID NO: 266; SEQ ID NO: 306 and SEQ ID NO: 267; SEQ ID NO: 306 and SEQ ID NO: 268; SEQ ID NO: 306 and SEQ ID NO: 269; SEQ ID NO: 306 and SEQ ID NO: 270; SEQ ID NO: 306 and SEQ ID NO: 271; SEQ ID NO: 306 and SEQ ID NO: 272; SEQ ID NO: 306 and SEQ ID NO: 273; SEQ ID NO: 306 and SEQ ID NO: 274; SEQ ID NO: 306 and SEQ ID NO: 275; SEQ ID NO: 306 and SEQ ID NO: 276; SEQ ID NO: 306 and SEQ ID NO: 277; SEQ ID NO: 306 and SEQ ID NO: 278; SEQ ID NO: 306 and SEQ ID NO: 279; SEQ ID NO: 306 and SEQ ID NO: 280; SEQ ID NO: 306 and SEQ ID NO: 281; SEQ ID NO: 306 and SEQ ID NO: 282; SEQ ID NO: 306 and SEQ ID NO: 283; SEQ ID NO: 306 and SEQ ID NO: 284; SEQ ID NO: 306 and SEQ ID NO: 285; SEQ ID NO: 306 and SEQ ID NO: 286; SEQ ID NO: 306 and SEQ ID NO: 287; SEQ ID NO: 306 and SEQ ID NO: 288; SEQ ID NO: 306 and SEQ ID NO: 289; SEQ ID NO: 306 and SEQ ID NO: 290; SEQ ID NO: 306 and SEQ ID NO: 291; SEQ ID NO: 306 and SEQ ID NO: 292; SEQ ID NO: 306 and SEQ ID NO: 293; SEQ ID NO: 306 and SEQ ID NO: 294; SEQ ID NO: 306 and SEQ ID NO: 295; SEQ ID NO: 306 and SEQ ID NO: 296; SEQ ID NO: 306 and SEQ ID NO: 297; and SEQ ID NO: 306 and SEQ ID NO: 298.

In some aspects, the CDR-H3-CDR-L3 pairs are selected from SEQ ID NO: 307 and SEQ ID NO: 240; SEQ ID NO: 307 and SEQ ID NO: 241; SEQ ID NO: 307 and SEQ ID NO: 242; SEQ ID NO: 307 and SEQ ID NO: 243; SEQ ID NO: 307 and SEQ ID NO: 244; SEQ ID NO: 307 and SEQ ID NO: 245; SEQ ID NO: 307 and SEQ ID NO: 246; SEQ ID NO: 307 and SEQ ID NO: 247; SEQ ID NO: 307 and SEQ ID NO: 248; SEQ ID NO: 307 and SEQ ID NO: 249; SEQ ID NO: 307 and SEQ ID NO: 250; SEQ ID NO: 307 and SEQ ID NO: 251; SEQ ID NO: 307 and SEQ ID NO: 252; SEQ ID NO: 307 and SEQ ID NO: 253; SEQ ID NO: 307 and SEQ ID NO: 254; SEQ ID NO: 307 and SEQ ID NO: 255; SEQ ID NO: 307 and SEQ ID NO: 256; SEQ ID NO: 307 and SEQ ID NO: 257; SEQ ID NO: 307 and SEQ ID NO: 258; SEQ ID NO: 307 and SEQ ID NO: 259; SEQ ID NO: 307 and SEQ ID NO: 260; SEQ ID NO: 307 and SEQ ID NO: 261; SEQ ID NO: 307 and SEQ ID NO: 262; SEQ ID NO: 307 and SEQ ID NO: 263; SEQ ID NO: 307 and SEQ ID NO: 264; SEQ ID NO: 307 and SEQ ID NO: 265; SEQ ID NO: 307 and SEQ ID NO: 266; SEQ ID NO: 307 and SEQ ID NO: 267; SEQ ID NO: 307 and SEQ ID NO: 268; SEQ ID NO: 307 and SEQ ID NO: 269; SEQ ID NO: 307 and SEQ ID NO: 270; SEQ ID NO: 307 and SEQ ID NO: 271; SEQ ID NO: 307 and SEQ ID NO: 272; SEQ ID NO: 307 and SEQ ID NO: 273; SEQ ID NO: 307 and SEQ ID NO: 274; SEQ ID NO: 307 and SEQ ID NO: 275; SEQ ID NO: 307 and SEQ ID NO: 276; SEQ ID NO: 307 and SEQ ID NO: 277; SEQ ID NO: 307 and SEQ ID NO: 278; SEQ ID NO: 307 and SEQ ID NO: 279; SEQ ID NO: 307 and SEQ ID NO: 280; SEQ ID NO: 307 and SEQ ID NO: 281; SEQ ID NO: 307 and SEQ ID NO: 282; SEQ ID NO: 307 and SEQ ID NO: 283; SEQ ID NO: 307 and SEQ ID NO: 284; SEQ ID NO: 307 and SEQ ID NO: 285; SEQ ID NO: 307 and SEQ ID NO: 286; SEQ ID NO: 307 and SEQ ID NO: 287; SEQ ID NO: 307 and SEQ ID NO: 288; SEQ ID NO: 307 and SEQ ID NO: 289; SEQ ID NO: 307 and SEQ ID NO: 290; SEQ ID NO: 307 and SEQ ID NO: 291; SEQ ID NO: 307 and SEQ ID NO: 292; SEQ ID NO: 307 and SEQ ID NO: 293; SEQ ID NO: 307 and SEQ ID NO: 294; SEQ ID NO: 307 and SEQ ID NO: 295; SEQ ID NO: 307 and SEQ ID NO: 296; SEQ ID NO: 307 and SEQ ID NO: 297; and SEQ ID NO: 307 and SEQ ID NO: 298.

2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.7.2. CDR-H1-CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of SEQ ID NOs: 4-62, and the CDR-L1 sequence is a CDR-L1 sequence comprising, consisting of, or consisting essentially of SEQ ID NOs: 299-301.

In some aspects, the CDR-H1 sequence is a Kabat CDR-H1 sequence comprising, consisting of, or consisting essentially of SEQ ID NOs: 63-121, and the CDR-L1 sequence is a CDR-L1 sequence comprising, consisting of, or consisting essentially of SEQ ID NOs: 299-301.

2.7.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.7.3. CDR-H2-CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of SEQ ID NOs: 122-180, and the CDR-L2 sequence is a CDR-L2 sequence comprising, consisting of, or consisting essentially of SEQ ID NOs: 302-304.

In some aspects, the CDR-H1 sequence is a Kabat CDR-H2 sequence comprising, consisting of, or consisting essentially of SEQ ID NOs: 181-239, and the CDR-L2 sequence is a CDR-L2 sequence comprising, consisting of, or consisting essentially of SEQ ID NOs: 302-304.

2.7.3.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

2.7.4. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOs: 308-366, and the V_{L} sequence is a V_{L} sequence comprising, consisting of, or consisting essentially of SEQ ID NOs: 367-369.

In some aspects, the V_{H}-V_{L} pairs are selected from SEQ ID NO: 367 and SEQ ID NO: 308; SEQ ID NO: 367 and SEQ ID NO: 309; SEQ ID NO: 367 and SEQ ID NO: 310; SEQ ID NO: 367 and SEQ ID NO: 311; SEQ ID NO: 367 and SEQ ID NO: 312; SEQ ID NO: 367 and SEQ ID NO: 313; SEQ ID NO: 367 and SEQ ID NO: 314; SEQ ID NO: 367 and SEQ ID NO: 315; SEQ ID NO: 367 and SEQ ID NO: 316; SEQ ID NO: 367 and SEQ ID NO: 317; SEQ ID NO: 367 and SEQ ID NO: 318; SEQ ID NO: 367 and SEQ ID NO: 319; SEQ ID NO: 367 and SEQ ID NO: 320; SEQ ID NO: 367 and SEQ ID NO: 321; SEQ ID NO: 367 and SEQ ID NO: 322; SEQ ID NO: 367 and SEQ ID NO: 323; SEQ ID NO: 367 and SEQ ID NO: 324; SEQ ID NO: 367 and SEQ ID NO: 325; SEQ ID NO: 367 and SEQ ID NO: 326; SEQ ID NO: 367 and SEQ ID NO: 327; SEQ ID NO: 367 and SEQ ID NO: 328; SEQ ID NO: 367 and SEQ ID NO: 329; SEQ ID NO: 367 and SEQ ID NO: 330; SEQ ID NO: 367 and SEQ ID NO: 331; SEQ ID NO: 367 and SEQ ID NO: 332; SEQ ID NO: 367 and SEQ ID NO: 333; SEQ ID NO: 367 and SEQ ID NO: 334; SEQ ID NO: 367 and SEQ ID NO: 335; SEQ ID NO: 367 and SEQ ID NO: 336; SEQ ID NO: 367 and SEQ ID NO: 337; SEQ ID NO: 367 and SEQ ID NO: 338; SEQ ID NO: 367 and SEQ ID NO: 339; SEQ ID NO: 367 and SEQ ID NO: 340; SEQ ID NO: 367 and SEQ ID NO: 341; SEQ ID NO: 367 and SEQ ID NO: 342; SEQ ID NO: 367 and SEQ ID NO: 343; SEQ ID NO: 367 and SEQ ID NO: 344; SEQ ID NO: 367 and SEQ ID NO: 345; SEQ ID NO: 367 and SEQ ID NO: 346; SEQ ID NO: 367 and SEQ ID NO: 347; SEQ ID NO: 367 and SEQ ID NO: 348; SEQ ID NO: 367 and SEQ ID NO: 349; SEQ ID NO: 367 and SEQ ID NO: 350; SEQ ID NO: 367 and SEQ ID NO: 351; SEQ ID NO: 367 and SEQ ID NO: 352; SEQ ID NO: 367 and SEQ ID NO: 353; SEQ ID NO: 367 and SEQ ID NO: 354; SEQ ID NO: 367 and SEQ ID NO: 355; SEQ ID NO: 367 and SEQ ID NO: 356; SEQ ID NO: 367 and SEQ ID NO: 357; SEQ ID NO: 367 and SEQ ID NO: 358; SEQ ID NO: 367 and SEQ ID NO: 359; SEQ ID NO: 367 and SEQ ID NO: 360; SEQ ID NO: 367 and SEQ ID NO: 361; SEQ ID NO: 367 and SEQ ID NO: 362; SEQ ID NO: 367 and SEQ ID NO: 363; SEQ ID NO: 367 and SEQ ID NO: 364; SEQ ID NO: 367 and SEQ ID NO: 365; and SEQ ID NO: 367 and SEQ ID NO: 366.

In some aspects, the V_{H}-V_{L} pairs are selected from SEQ ID NO: 368 and SEQ ID NO: 308; SEQ ID NO: 368 and SEQ ID NO: 309; SEQ ID NO: 368 and SEQ ID NO: 310; SEQ ID NO: 368 and SEQ ID NO: 311; SEQ ID NO: 368 and SEQ ID NO: 312; SEQ ID NO: 368 and SEQ ID NO: 313; SEQ ID NO: 368 and SEQ ID NO: 314; SEQ ID NO: 368 and SEQ ID NO: 315; SEQ ID NO: 368 and SEQ ID NO: 316; SEQ ID NO: 368 and SEQ ID NO: 317; SEQ ID NO: 368 and SEQ ID NO: 318; SEQ ID NO: 368 and SEQ ID NO: 319; SEQ ID NO: 368 and SEQ ID NO: 320; SEQ ID NO: 368 and SEQ ID NO: 321; SEQ ID NO: 368 and SEQ ID NO: 322; SEQ ID NO: 368 and SEQ ID NO: 323; SEQ ID NO: 368 and SEQ ID NO: 324; SEQ ID NO: 368 and SEQ ID NO: 325; SEQ ID NO: 368 and SEQ ID NO: 326; SEQ ID NO: 368 and SEQ ID NO: 327; SEQ ID NO: 368 and SEQ ID NO: 328; SEQ ID NO: 368 and SEQ ID NO: 329; SEQ ID NO: 368 and SEQ ID NO: 330; SEQ ID NO: 368 and SEQ ID NO: 331; SEQ ID NO: 368 and SEQ ID NO: 332; SEQ ID NO: 368 and SEQ ID NO: 333; SEQ ID NO: 368 and SEQ ID NO: 334; SEQ ID NO: 368 and SEQ ID NO: 335; SEQ ID NO: 368 and SEQ ID NO: 336; SEQ ID NO: 368 and SEQ ID NO: 337; SEQ ID NO: 368 and SEQ ID NO: 338; SEQ ID NO: 368 and SEQ ID NO: 339; SEQ ID NO: 368 and SEQ ID NO: 340; SEQ ID NO: 368 and SEQ ID NO: 341; SEQ ID NO: 368 and SEQ ID NO: 342; SEQ ID NO: 368 and SEQ ID NO: 343; SEQ ID NO: 368 and SEQ ID NO: 344; SEQ ID NO: 368 and SEQ ID NO: 345; SEQ ID NO: 368 and SEQ ID NO: 346; SEQ ID NO: 368 and SEQ ID NO: 347; SEQ ID NO: 368 and SEQ ID NO: 348; SEQ ID NO: 368 and SEQ ID NO: 349; SEQ ID NO: 368 and SEQ ID NO: 350; SEQ ID NO: 368 and SEQ ID NO: 351; SEQ ID NO: 368 and SEQ ID NO: 352; SEQ ID NO: 368 and SEQ ID NO: 353; SEQ ID NO: 368 and SEQ ID NO: 354; SEQ ID NO: 368 and SEQ ID NO: 355; SEQ ID NO: 368 and SEQ ID NO: 356; SEQ ID NO: 368 and SEQ ID NO: 357; SEQ ID NO: 368 and SEQ ID NO: 358; SEQ ID NO: 368 and SEQ ID NO: 359; SEQ ID NO: 368 and SEQ ID NO: 360; SEQ ID NO: 368 and SEQ ID NO: 361; SEQ ID NO: 368 and SEQ ID NO: 362; SEQ ID NO: 368 and SEQ ID NO: 363; SEQ ID NO: 368 and SEQ ID NO: 364; SEQ ID NO: 368 and SEQ ID NO: 365; and SEQ ID NO: 368 and SEQ ID NO: 366.

In some aspects, the V_{H}-V_{L} pairs are selected from SEQ ID NO: 369 and SEQ ID NO: 308; SEQ ID NO: 369 and SEQ ID NO: 309; SEQ ID NO: 369 and SEQ ID NO: 310; SEQ ID NO: 369 and SEQ ID NO: 311; SEQ ID NO: 369 and SEQ ID NO: 312; SEQ ID NO: 369 and SEQ ID NO: 313; SEQ ID NO: 369 and SEQ ID NO: 314; SEQ ID NO: 369 and SEQ ID NO: 315; SEQ ID NO: 369 and SEQ ID NO: 316; SEQ ID NO: 369 and SEQ ID NO: 317; SEQ ID NO: 369 and SEQ ID NO: 318; SEQ ID NO: 369 and SEQ ID NO: 319; SEQ ID NO: 369 and SEQ ID NO: 320; SEQ ID NO: 369 and SEQ ID NO: 321; SEQ ID NO: 369 and SEQ ID NO: 322; SEQ ID NO: 369 and SEQ ID NO: 323; SEQ ID NO: 369 and SEQ ID NO: 324; SEQ ID NO: 369 and SEQ ID NO: 325; SEQ ID NO: 369 and SEQ ID NO: 326; SEQ ID NO: 369 and SEQ ID NO: 327; SEQ ID NO: 369 and SEQ ID NO: 328; SEQ ID NO: 369 and SEQ ID NO: 329; SEQ ID NO: 369 and SEQ ID NO: 330; SEQ ID NO: 369 and SEQ ID NO: 331; SEQ ID NO: 369 and SEQ ID NO: 332; SEQ ID NO: 369 and SEQ ID NO: 333; SEQ ID NO: 369 and SEQ ID NO: 334; SEQ ID NO: 369 and SEQ ID NO: 335; SEQ ID NO: 369 and SEQ ID NO: 336; SEQ ID NO: 369 and SEQ ID NO: 337; SEQ ID NO: 369 and SEQ ID NO: 338; SEQ ID NO: 369 and SEQ ID NO: 339; SEQ ID NO: 369 and SEQ ID NO: 340; SEQ ID NO: 369 and SEQ ID NO: 341; SEQ ID NO: 369 and SEQ ID NO: 342; SEQ ID NO: 369 and SEQ ID NO: 343; SEQ ID NO: 369 and SEQ ID NO: 344; SEQ ID NO: 369 and SEQ ID NO: 345; SEQ ID NO: 369 and SEQ ID NO: 346; SEQ ID NO: 369 and SEQ ID NO: 347; SEQ ID NO: 369 and SEQ ID NO: 348; SEQ ID NO: 369 and SEQ ID NO: 349; SEQ ID NO: 369 and SEQ ID NO: 350; SEQ ID NO: 369 and SEQ ID NO: 351; SEQ ID NO: 369 and SEQ ID NO: 352; SEQ ID NO: 369 and SEQ ID NO: 353; SEQ ID NO: 369 and SEQ ID NO: 354; SEQ ID NO: 369 and SEQ ID NO: 355; SEQ ID NO: 369 and SEQ ID NO: 356; SEQ ID NO: 369 and SEQ ID NO: 357; SEQ ID NO: 369 and SEQ ID NO: 358; SEQ ID NO: 369 and SEQ ID NO: 359; SEQ ID NO: 369 and SEQ ID NO: 360; SEQ ID NO: 369 and SEQ ID NO: 361; SEQ ID NO: 369 and SEQ ID NO: 362; SEQ ID NO: 369 and SEQ ID NO: 363; SEQ ID NO: 369 and SEQ ID NO: 364; SEQ ID NO: 369 and SEQ ID NO: 365; and SEQ ID NO: 369 and SEQ ID NO: 366.

2.7.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the Vii sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative Vii sequences provided in this disclosure.

In some embodiments, the V_{H} sequence comprises, consists of, or consists essentially of any of the illustrative Vii sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.8. Antibodies Comprising All Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 4-62; the CDR-H2 sequence is a Chothia CDR-H2 sequence comprising, consisting of, or consisting essentially of SEQ ID NOs: 122-180; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

In some aspects, the CDR-H1 sequence is a Kabat CDR-H1 sequence comprising, consisting of, or consisting essentially of SEQ ID NOs: 63-121; the CDR-H2 sequence is a Kabat CDR-H2 sequence comprising, consisting of, or consisting essentially of SEQ ID NOs: 181-239; the CDR-H3 sequence is a CDR-H3 sequence comprising, consisting of, or consisting essentially of SEQ ID NOs: 240-298; the CDR-L1 sequence is a CDR-L1 sequence comprising, consisting of, or consisting essentially of SEQ ID NOs: 299-301; the CDR-L2 sequence is a CDR-L2 sequence comprising, consisting of, or consisting essentially of SEQ ID NOs: 302-304; and the CDR-L3 sequence is a CDR-L3 sequence comprising, consisting of, or consisting essentially of SEQ ID NOs: 305-307.

2.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.9. Consensus Sequences

In some embodiments, provided herein are anti-FOLR1 antibodies comprising one or more sequences defined by consensus sequences. Each consensus sequence is based, at least in part, on one or more alignments of two or more useful anti-FOLR1 CDR sequences provided in this disclosure. Based on such alignments, a person of skill in the art would recognize that different amino acid residues may useful in certain positions of the CDRs. Accordingly, each consensus sequence encompasses two or more useful anti-FOLR1 CDR sequences.

In some embodiments, the antibodies comprise one to six of the consensus CDR sequences provided herein. In some embodiments, the antibodies comprise two to six of the consensus CDR sequences provided herein. In some embodiments, the antibodies comprise three to six of the consensus CDR sequences provided herein. In some embodiments, the antibodies comprise four to six of the consensus CDR sequences provided herein. In some embodiments, the antibodies comprise five to six of the consensus CDR sequences provided herein. In some embodiments, the antibodies comprise six of the consensus CDR sequences provided herein. In some embodiments, the antibodies comprise a V_{L} comprising the CDR-L consensus sequence(s). In some embodiments, the antibodies comprise a V_{H} comprising the CDR-H consensus sequence(s). In some embodiments, the antibodies comprise a V_{H} comprising the CDR-H consensus sequence(s) and a V_{L} comprising the CDR-L consensus sequence(s).

2.9.1. CDR-H3 Consensus Sequences

In some embodiments, the antibody comprises a CDR-H3 sequence defined by the consensus sequence G-α₂-α₃-α₄-W-α₆-α₇-G-α₉-α₁₀-Y-α₁₂-α₁₃-α₁₄-Y, where α₂ is G, S, A, F, H, R, T, or Y; α₃ is W, L, or Y; α₄ is S, A, F, Y, H, or D; as is R, P, Q, or K; α₇ is S, A, or H; α₉ is Y, H, or M; α₁₀ is G, S, D, or W; α₁₂ is Y or F; α₁₃ is L, I, Q, or M; and α₁₄ is D or E.

In some embodiments, the antibody comprises a CDR-H3 sequence defined by the consensus sequence G-α₂-α₃-α₄-W-α₆-α₇-G-α₉-α₁₀-Y-α₁₂-α₁₃-α₁₄-Y, where α₂ is G or S; α₃ is W; α₄ is S or H; as is R or P; α₇ is S; α₉ is Y or M; α₁₀ is G, S, or D; α₁₂ is Y; α₁₃ is L; and α₁₄ is D.

2.9.2. Chothia CDR-H1 Consensus Sequences

In some embodiments, the antibody comprises a Chothia CDR-H1 sequence defined by the consensus sequence γ₁-γ₂-γ₃-γ₄-γ₅-γ₆-γ₇, where γ₁ is G or S; γ₂ is F or S; γ₃ is N; γ₄ is I or T; γ₅ is S, R, G, T, N, or D; γ₆ is N, K, T, R, H, Y, L, M, Q, or V; and γ₇ is Y, H, S, N, K, F, or Q.

In some embodiments, the antibody comprises a Chothia CDR-H1 sequence defined by the consensus sequence γ₁-γ₂-γ₃-γ₄-γ₅-γ₆-γ₇, where γ₁ is G; γ₂ is F; γ₃ is N; γ₄ is I or T; γ₅ is S, R, or T; γ₆ is N or T; and γ₇ is Y, K, or Q.

2.9.3. Chothia CDR-H2 Consensus Sequences

In some embodiments, the antibody comprises a Chothia CDR-H2 sequence defined by the consensus sequence ε₁-ε₂-ε₃-ε₄-ε₅-ε₆, where ε₁ is Y, T, F, S, or A; ε₂ is P; ε₃ is N, I, V, R, Y, F, G, L, Q, or S; ε₄ is D or P; ε₅ is G or D; and ε₆ is Y, I, T, N, F, S, or M.

In some embodiments, the antibody comprises a Chothia CDR-H2 sequence defined by the consensus sequence ε₁-ε₂-ε₃-ε₄-ε₅-ε₆, where ε₁ is Y or F; ε₂ is P; ε₃ is N, I, or R; ε₄ is D; ε₅ is G; and ε₆ is Y or I.

2.9.4. Kabat CDR-H1 Consensus Sequences

In some embodiments, the antibody comprises a Kabat CDR-H1 sequence defined by the consensus sequence ζ₁-ζ₂-ζ₃-ζ₄-ζ₅, where ζ₁ is N, K, T, R, H, Y, L, M, Q, or V; ζ₂ is Y, H, S, N, K, F, or Q; ζ₃ is S or Y; ζ₄ is I; and ζ₅ is H.

In some embodiments, the antibody comprises a Kabat CDR-H1 sequence defined by the consensus sequence ζ₁-ζ₂-ζ₃-ζ₄-ζ₅, where ζ₁ is N or T; ζ₂ is Y, K, or Q; ζ₃ is S; ζ₄ is I; and ζ₅ is H.

2.9.5. Kabat CDR-H2 Consensus Sequences

In some embodiments, the antibody comprises a Kabat CDR-H2 sequence defined by the consensus sequence θ₁-θ₂-θ₃-θ₄-θ₅-θ₆-θ₇-θ₈-θ₉-D-Y-A-D-θ₁₄-θ₁₅-θ₁₆-G, where θ₁ is G, E, D, W, S, or V; θ₂ is I or V; θ₃ is Y, T, F, S, or A; θ₄ is P; θ₅ is N, I, V, R, Y, F, G, L, Q, or S; θ₆ is D or P; θ₇ is G or D; θ₈ is Y, I, T, N, F, S, or M; θ₉ is T or N; θ₁₄ is S, R, or N; θ₁₅ is V or M; and θ₁₆ is K or E.

In some embodiments, the antibody comprises a Kabat CDR-H2 sequence defined by the consensus sequence θ₁-θ₂-θ₃-θ₄-θ₅-θ₆-θ₇-θ₈-θ₉-D-Y-A-D-θ₁₄-θ₁₅-θ₁₆-G, where θ₁ is G, E, or D; θ₂ is I; θ₃ is Y or F; θ₄ is P; θ₅ is N, I, or R; θ₆ is D; θ₇ is G; θ₈ is Y or I; θ₉ is T; θ₁₄ is S; θ₁₅ is V; and θ₁₆ is K.

### 3. Germline

In some embodiments, the antibody that specifically binds folate receptor alpha is an antibody comprising a variable region that is encoded by a particular germline gene, or a variant thereof. The illustrative antibodies provided herein comprise variable regions that are encoded by the heavy chain variable region germline genes VH1-18, VH3-33 VH2-5, V_{H}2-70, and VH4-30-4. or variants thereof; and the light chain variable region germline genes Vκ1-5, Vκ3-11, Vκ2-20, Vκ1-33, and Vκ1-16, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H}1, V_{H}2, V_{H}3, or V_{H}4 families, or a variant thereof. In some embodiments, a CDR-L sequence provided herein may be combined with a variable region encoded by a variable region germline gene selected from the Vκ1, Vκ2, or Vκ3, or a variant thereof.

### 4. Affinity

In some embodiments, the affinity of the antibody for folate receptor alpha as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻⁹ M and 10⁻¹⁰ M.

In some embodiments, the affinity of the antibody for human folate receptor alpha, as determined by surface plasmon resonance at 25° C., and as indicated by K_{D}, is from about 0.36×10⁻⁹ M to about 2.21×10⁻⁹ M. In some embodiments, the affinity of the antibody for human folate receptor alpha, as determined by surface plasmon resonance at 25° C., and as indicated by K_{D}, is from about 8.55×10⁻¹⁰ M to about 1.70×10⁻⁸ M. In some embodiments, the affinity of the antibody for human folate receptor alpha, as determined by surface plasmon resonance at 25° C., and as indicated by K_{D}, is from about 5.71×10⁻¹⁰ M to about 2.58×10⁻⁸ M. In some embodiments, the affinity of the antibody for human folate receptor alpha is about any of the K_{D} values reported for human folate receptor alpha in the examples below.

In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of at least about 10⁷ M⁻¹×sec⁻¹. In some embodiments the antibody has a kₐ of at least about 10⁸ M⁻¹×sec⁻¹. In some embodiments the antibody has a kₐ of at least about 10⁹ M⁻¹×sec⁻¹. In some embodiments the antibody has a kₐ of between about 10⁴ M⁻¹×sec⁻¹ and about 10¹⁰ M⁻¹×sec⁻¹. In some embodiments the antibody has a kₐ of between about 10⁵ M⁻¹×sec⁻¹ and about 10¹⁰ M⁻¹×sec⁻¹. In some embodiments the antibody has a kₐ of between about 10⁶ M⁻¹×sec⁻¹ and about 10¹⁰ M⁻¹×sec⁻¹. In some embodiments the antibody has a kₐ of between about 10⁷ M⁻¹×sec⁻¹ and about 10¹⁰ M⁻¹×sec⁻¹.

In some embodiments the antibody has a kₐ when associating with human folate receptor alpha, as determined by surface plasmon resonance at 25° C., of from about 4.44×10⁵ M⁻¹×sec⁻¹ to about 1.61×10⁵ M⁻¹×sec⁻¹. In some embodiments the antibody has a kₐ when associating with human folate receptor alpha, as determined by surface plasmon resonance at 25° C., of from about 2.90×10⁵ M⁻¹×sec⁻¹ to about 9.64×10⁹ M⁻¹×sec⁻¹. In some embodiments the antibody has a kₐ when associating with human folate receptor alpha of about any of the kₐ values reported for human folate receptor alpha in the examples below.

In some embodiments the antibody has a k_{d} of about 10⁻⁵ sec⁻¹ or less. In some embodiments the antibody has a k_{d} of about 10⁻⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 10⁻⁴ sec⁻¹. In some embodiments the antibody has a k_{d} of between about 10⁻³ sec⁻¹ and about 10⁻⁵ sec⁻¹.

In some embodiments the antibody has a k_{d} when dissociating from human folate receptor alpha, as determined by surface plasmon resonance at 25° C., of from about 8.66×10⁻⁴ sec⁻¹ to about 1.08×10⁻² sec⁻¹. In some embodiments the antibody has a k_{d} when dissociating from human folate receptor alpha, as determined by surface plasmon resonance at 25° C., of from about 2.28×10⁻⁴ sec⁻¹ to about 4.82×10¹ sec⁻¹. In some embodiments the antibody has a k_{d} when dissociating from human folate receptor alpha of about any of the k_{d} values reported for human folate receptor alpha in the examples below.

In some embodiments, the affinity of the antibody for cynomolgus folate receptor alpha, as determined by surface plasmon resonance at 25° C., and as indicated by K_{D}, is from about 0.19×10⁻⁹ M to about 2.84×10⁻⁹ M. In some embodiments, the affinity of the antibody for cynomolgus folate receptor alpha is about any of the K_{D} values reported for cynomolgus folate receptor alpha in the examples below.

In some embodiments, the affinity of the antibody for mouse folate receptor alpha, as determined by surface plasmon resonance at 25° C., and as indicated by K_{D}, is from about 0.5×10⁻⁹ M to about 9.07×10⁻⁸ M. In some embodiments, the affinity of the antibody for mouse folate receptor alpha is about any of the K_{D} values reported for mouse folate receptor alpha in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 5. Epitope Bins

In some embodiments, the antibody binds the same epitope as an antibody encompassing any of SEQ ID NOs: 308-366. In some embodiments, the antibody binds the same epitope as an antibody comprising (a) a V_{H} sequence comprising, consisting or, or consisting essentially of SEQ ID NOs: 308-366, and (b) a V_{L} sequence comprising, consisting of, or consisting essentially of SEQ ID NOs: 367-369. For example, in some embodiments, the antibody binds the same epitope as an antibody comprising any of the V_{H}-V_{L} pairs, above. In some embodiments, the antibody competes for epitope binding with an antibody encompassing any of SEQ ID NOs: 308-366. In some embodiments, the antibody competes for epitope binding with an antibody comprising (a) a V_{H} sequence comprising, consisting or, or consisting essentially of SEQ ID NOs: 308-366, and (b) a V_{L} sequence comprising, consisting of, or consisting essentially of SEQ ID NOs: 367-369. For example, in some embodiments, the antibody competes for epitope binding with an antibody comprising any of the V_{H}-V_{L} pairs, above.

### 6. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 7. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the C_{H}3 sequences. In some embodiments, the Fc comprises one or more modifications in at least one of the C_{H}2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 8. Preparation of Antibodies

8.1. Antigen Preparation

The FOLR1 protein to be used for isolation of the antibodies may be intact FOLR1 or a fragment of FOLR1. The intact FOLR1 protein, or fragment of FOLR1, may be in the form of an isolated protein or protein expressed by a cell. Other forms of FOLR1 useful for generating antibodies will be apparent to those skilled in the art.

8.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

8.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

8.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 9. Vectors, Host Cells, and Recombinant Methods

Embodiments are also directed to the provision of isolated nucleic acids encoding anti-FOLR1 antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-FOLR1 antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-FOLR1 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 10. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition provided herein is administered parenterally.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In some embodiments lactose-free compositions are provided herein which comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody will decompose. Such excipients, which are referred to herein as “stabilizers,” include, but are not limited to, antioxidants such as ascorbic acid, pH buffers, or salt buffers.

10.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

10.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

11. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving folate receptor alpha (FOLR1). In some embodiments, the disease or condition is a disease or condition that can be diagnosed by overexpression of folate receptor alpha. In some embodiments, the disease or condition is a disease or condition that can benefit from treatment with an anti-folate receptor alpha antibody. In some embodiments, the disease or condition is a cancer.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibodies provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is triple-negative breast cancer (TNBC). In some embodiments, the disease is lung cancer. In some embodiments, the disease is non-small cell lung cancer (NSCLC). In some embodiments, the disease is head and neck cancer. In some embodiments, the disease is renal cell carcinoma. In some embodiments, the disease is brain carcinoma. In some embodiments, the disease is endometrial cancer.

### 12. Diagnostic Applications

In some embodiments, the antibodies provided herein are used in diagnostic applications. For example, an anti-FOLR1 antibody may be useful in assays for FOLR1 protein. In some aspects the antibody can be used to detect the expression of FOLR1 in various cells and tissues. These assays may be useful, for example, in making a diagnosis and/or prognosis for a disease, such as a cancer.

In some diagnostic and prognostic applications, the antibody may be labeled with a detectable moiety. Suitable detectable moieties include, but are not limited to radioisotopes, fluorescent labels, and enzyme-substrate labels. In another embodiment, the anti-FOLR1 antibody need not be labeled, and the presence of the antibody can be detected using a labeled antibody which specifically binds to the anti-FOLR1 antibody.

### 13. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the folate receptor alpha protein (or fragment thereof) to be purified, and thereafter the support is washed with a suitable solvent that will remove substantially all the material in the sample except the folate receptor alpha protein, which is bound to the immobilized antibody. Finally, the support is washed with another suitable solvent, such as glycine buffer, pH 5.0 that will release the folate receptor alpha protein from the antibody.

### 14. Kits

In some embodiments, an anti-FOLR1 antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-FOLR1 antibody. In some embodiments, the anti-FOLR1 antibody is provided in the form of a pharmaceutical composition.

### EXAMPLES

### Example 1

### Generation and Primary Screening of Anti-FOLR1 Antibodies

Antibody Fab libraries were constructed using a standard overlap extension PCR protocol with mutagenic primers targeting complementary determining regions (CDRs). See Heckman and Pease, Nat. Protoc., 2007, 2:924-932; Stafford et al., 2014, Protein Eng. Des. Sel. 27:97-109, both incorporated by reference in their entireties. Selections for novel antibodies were performed using standard ribosome display protocols. See Dreier and Plückthun, 2011, Methods Mol Biol 687:283-306, which is incorporated herein by reference in its entirety.

Initial antibody leads from ribosome display were derived from a naïve human library which was constructed by overlapping PCR using trastuzumab HC as the base template. CDRs H1 and H2 were randomized with the same design as described by Lee et al., J. Mol. Biol. 2004, 340:1073-1093 using oligonucleotides purchased from Integrated DNA Technologies. In this design, CDRs H1 and H2 closely match the observed amino acid distributions of natural human antibodies. CDR H3 was diversified using oligonucleotides incorporating trimer phosphoramidite mixtures (TRIMs) for amino acid randomization. The TRIM oligos were synthesized as described by Yagodkin A et al., Nucleosides Nucleotides Nucleic Acids 2007, 26:473-97. Specifically, six separate oligonucleotides containing TRIMs were used to make 6 separate H3 loop-lengths (13-18; as defined by Zemlin et al.) to match the most common loop lengths observed in the human repertoire. Together these loop lengths comprise approximately 54.5% of the naturally-occurring loop length variation in human IgGs as reported by Zemlin et al., J. Mol. Biol. 2003, 334:733-749. The frequency distribution of each amino acid was designed to closely match the observed distribution of amino acids in CDR H3 of human IgGs as reported by Zemlin et al. Altogether, the library closely matches natural human antibody variation which is known in the field to improve antibody stability and folding of antibodies as described by Zhai et al., J Mol Biol. 2011, 412:55-71. The heavy chain (HC) library was paired with a constant, unmodified trastuzumab light chain (LC) throughout the selection process as described by Stafford et al., Protein Eng Des Sel 2014, 27:97-109.

Affinity maturated antibody leads (e.g., SRP 1848 antibodies, below) were derived from a focused library, biased towards two leads, which was constructed by overlapping PCR using “soft-randomized” oligonucleotides purchased from Eurofins MWG Operon. Soft-randomization is a process in which a biased distribution of nucleotides is used for each soft-randomized codon such that the parent amino acid sequence is coded more frequently than other amino acids-30% of the time. Other amino acids are coded at each position but at a lower percentage. At each soft-randomized position, 70% of the parent nucleotide is mixed with 10% of the other three nucleotides. For the library, CDRs H1, H2, and H3 were soft-randomized simultaneously and selected by standard ribosome display protocols. As with the selection of initial leads, the affinity matured antibodies were paired with a constant, unmodified trastuzumab LC throughout the selection process as described by Stafford et al., Protein Eng Des Sel 2014, 27:97-109.

Selections for novel antibodies were performed using standard ribosome display protocols. See Dreier and Plückthun, Methods Mol. Biol., 2003, 687:283-306, Clifton, N.J., incorporated by reference in its entirety. Fab ribosome display selections were performed according to published protocols. See Stafford et al., 2014, Protein Eng. Des. Sel. 27:97-109; Hanes and Plückthun, Proc. Natl. Acad. Sci. U.S.A., 1997, 94:4937-4942; both incorporated by reference in their entireties. After multiple rounds of selection, the DNA from RT-PCR output was cloned into an optimized vector for cell-free expression using standard molecular biology techniques. See Yin et al., mAbs, 2012, 4:217-225, incorporated by reference in its entirety. All constructs were HIS- and FLAG-tagged to streamline purification and testing during screening.

Libraries of antibody variants generated by selection workflow were transformed into E. coli and grown on agar plates with antibiotic (kanamycin). Individual colonies were grown in liquid broth (TB+kanamycin), and used as a template for DNA amplification via rolling circle amplification (RCA). The variants were then expressed in cell-free protein synthesis reactions as described in Yin et al., mAbs, 2012, 4:217-225.

Briefly, cell-free extracts were treated with 50 μM iodoacetamide for 30 min at room temperature (20° C.) and added to a premix containing cell-free components (see Cai et al., Biotechnol Prg, 2015, 3:823-831, incorporated by reference in its entirety) and 10% (v/v) RCA DNA template (approximately 10 μg/mL DNA) for HC variants, in addition to 2.5 μg/mL Trastuzumab LC which is present for antibody assembly but is not varied in the library. Sixty microliters of cell-free reactions were incubated at 30° C. for 12 hr on a shaker at 650 rpm in 96-well plates. Four hundred to one-thousand-five-hundred (400 to 1500) colonies were screened, depending on the predicted diversity of different selection campaigns.

Following synthesis, each reaction was diluted 1:50 into PBS (pH 7.4) with 3% fetal bovine serum (FBS), and expressed variants were tested for functional activity via cell-based ELISA binding to CHO-hFOLR1 cells (human FOLR1 expressed recombinantly in Chinese Hamster Ovary cells). Briefly, 384-well plates were seeded with CHO-control or CHO-hFOLR1 cells the day before the assay. On the day of the assay, cells were fixed with 20 uL of 4% paraformaldehyde in PBS for 15 minutes in the dark, washed with PBS, and then blocked with 30% FBS in PBS for 30 minutes at room temperature. Antibody variants of interest (1:50 diluted cell-free reaction) were allowed to bind to the fixed CHO-hFOLR1 cells, and detected with secondary antibodies (e.g. HRP-conjugated Anti-human Fc or anti-FLAG) and then detected with chemiluminescent substrate (Pierce ELISA SuperSignal™ Substrate). Chemiluminescence was quantified on a Molecular Devices SpectraMax® M5 plate reader. Top hits were selected based on cell-based ELISA signal/noise ratio, and their nucleotides were sequenced. Based on binding activity and sequence analysis, a subset of variants was selected for further scale-up and characterization.

The top leads from ELISA-based screening were cultured, and plasmid minipreps were performed using a QIAprep® 96 Turbo miniprep kit (Qiagen) according to the manufacturer's instructions. 10 μg/mL miniprepped DNA was added to 4 mL cell-free reactions and incubated overnight for 12 hr at 30° C., at 650 rpm. In the case of IgG variants with a common Trastuzumab LC, 7.5 ug/mL of the HC variant DNA and 2.5 ug/mL of the common Trastuzumab LC were added to the reaction.

Expressed variants from clarified cell-free reactions were purified via immobilized metal ion affinity chromatography (IMAC) purification using a semi-automated high throughput batch purification method. Briefly, purifications were performed in a 96-well plate format where 50 jiL/well of IMAC resin (Ni Sepharose High Performance, GE Healthcare) was equilibrated in IMAC binding buffer (50 mM Tris pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM Tris pH 8.0, 300 mM NaCl, 500 mM imidazole) and buffer exchanged into PBS using a 96-well Zeba plate (7 kD MWCO, Thermo Fisher). Purified antibodies were quantified via high throughput capillary electrophoresis using the LabChip GXII (Perkin Elmer) against a Herceptin standard curve, according to the manufacturer's instructions.

Exemplary affinity-matured antibodies are reported in Table 5, below.

TABLE 5
Affinity Matured (SRP1848) Antibodies

| | | SEQ ID | | SEQ ID |
|---|---|---|---|---|
| Antibody | VH | NO. | VL | NO. |
| 1 | SRP1848-A01 | 308 | Trastuzumab | 367 |
| 2 | SRP1848-A02 | 309 | Trastuzumab | 367 |
| 3 | SRP1848-A04 | 310 | Trastuzumab | 367 |
| 4 | SRP1848-A06 | 311 | Trastuzumab | 367 |
| 5 | SRP1848-A07 | 312 | Trastuzumab | 367 |
| 6 | SRP1848-A08 | 313 | Trastuzumab | 367 |
| 7 | SRP1848-A09 | 314 | Trastuzumab | 367 |
| 8 | SRP1848-A10 | 315 | Trastuzumab | 367 |
| 9 | SRP1848-B01 | 316 | Trastuzumab | 367 |
| 10 | SRP1848-B03 | 317 | Trastuzumab | 367 |
| 11 | SRP1848-B04 | 318 | Trastuzumab | 367 |
| 12 | SRP1848-B05 | 319 | Trastuzumab | 367 |
| 13 | SRP1848-B06 | 320 | Trastuzumab | 367 |
| 14 | SRP1848-B07 | 321 | Trastuzumab | 367 |
| 15 | SRP1848-B09 | 322 | Trastuzumab | 367 |
| 16 | SRP1848-B10 | 323 | Trastuzumab | 367 |
| 17 | SRP1848-B11 | 324 | Trastuzumab | 367 |
| 18 | SRP1848-C01 | 325 | Trastuzumab | 367 |
| 19 | SRP1848-C03 | 326 | Trastuzumab | 367 |
| 20 | SRP1848-C04 | 327 | Trastuzumab | 367 |
| 21 | SRP1848-C05 | 328 | Trastuzumab | 367 |
| 22 | SRP1848-C07 | 329 | Trastuzumab | 367 |
| 23 | SRP1848-C10 | 330 | Trastuzumab | 367 |
| 24 | SRP1848-D02 | 331 | Trastuzumab | 367 |
| 25 | SRP1848-D03 | 332 | Trastuzumab | 367 |
| 26 | SRP1848-D04 | 333 | Trastuzumab | 367 |
| 27 | SRP1848-D05 | 334 | Trastuzumab | 367 |
| 28 | SRP1848-D07 | 335 | Trastuzumab | 367 |
| 29 | SRP1848-D09 | 336 | Trastuzumab | 367 |
| 30 | SRP1848-D10 | 337 | Trastuzumab | 367 |
| 31 | SRP1848-E01 | 338 | Trastuzumab | 367 |
| 32 | SRP1848-E02 | 339 | Trastuzumab | 367 |
| 33 | SRP1848-E03 | 340 | Trastuzumab | 367 |
| 34 | SRP1848-E05 | 341 | Trastuzumab | 367 |
| 35 | SRP1848-E06 | 342 | Trastuzumab | 367 |
| 36 | SRP1848-E07 | 343 | Trastuzumab | 367 |
| 37 | SRP1848-F01 | 344 | Trastuzumab | 367 |
| 38 | SRP1848-F02 | 345 | Trastuzumab | 367 |
| 39 | SRP1848-F04 | 346 | Trastuzumab | 367 |
| 40 | SRP1848-F05 | 347 | Trastuzumab | 367 |
| 41 | SRP1848-F06 | 348 | Trastuzumab | 367 |
| 42 | SRP1848-F07 | 349 | Trastuzumab | 367 |
| 43 | SRP1848-F08 | 350 | Trastuzumab | 367 |
| 44 | SRP1848-F09 | 351 | Trastuzumab | 367 |
| 45 | SRP1848-F10 | 352 | Trastuzumab | 367 |
| 46 | SRP1848-F11 | 353 | Trastuzumab | 367 |
| 47 | SRP1848-G01 | 354 | Trastuzumab | 367 |
| 48 | SRP1848-G03 | 355 | Trastuzumab | 367 |
| 49 | SRP1848-G04 | 356 | Trastuzumab | 367 |
| 50 | SRP1848-G06 | 357 | Trastuzumab | 367 |
| 51 | SRP1848-G07 | 358 | Trastuzumab | 367 |
| 52 | SRP1848-G09 | 359 | Trastuzumab | 367 |
| 53 | SRP1848-G10 | 360 | Trastuzumab | 367 |
| 54 | SRP1848-G11 | 361 | Trastuzumab | 367 |
| 55 | SRP1848-H01 | 362 | Trastuzumab | 367 |

### Example 2

### Preparation of SCFVS

A single-chain antibody is made in either the V_{H}V_{L} or V_{L}V_{H} orientation with a linker sequence between the V_{H} and V_{L} domains. Typically scFv linkers are composed of (GGGGS)n repeats where n=3, 4, 5, or 6 for linkers of 15, 20, 25, or 30 residues respectively. For cell-free expression, an N-terminal Met is added, but for mammalian expression a leader peptide is added. On the C-terminal end of the scFv, an Fc sequence can be added to extend in vivo half-life or the scFv can be used directly. An optional linker sequence can be incorporated between the scFv and the Fc. An exemplary scFv-Fc linker sequence is AAGSDQEPKSS (SEQ ID NO: 378). C-terminal affinity tags can optionally be added to facilitate purification and assay development. An exemplary affinity tag is a C-terminal FlagHis tag GSGDYKDDDDKGSGHHHHHH (SEQ ID NO: 376). A stop codon is typically inserted at the end of the sequence. An exemplary scFv can include an N-terminal Met residue, a V_{H} domain, a GGGGSGGGGSGGGGS (SEQ ID NO: 377) linker, a V_{L} domain, an AAGSDQEPKSS (SEQ ID NO: 378) linker, an Fc domain, a FlagHis tag, and a stop codon.

### Example 3

### Affinity and Kinetic Binding Analyses

Anti-Fc polyclonal antibodies were immobilized onto a CM5 chip (GE Life Sciences) using amine coupling chemistry (from Amine Coupling Kit, GE Life Sciences). The immobilization steps were carried out at a flow rate of 25 μL/min in 1×HBS-EP+buffer (GE Life Sciences; 10× Stock diluted before use). The sensor surfaces were activated for 7 min with a mixture of N-hydroxysuccinimide (NHS, 0.05 M) and 1-ethyl-3-(3-dimethylaminopropyl)-carbodiimide (EDC, 0.2 M). The anti-Fc polyclonal antibodies were injected over all 4 flow cells at a concentration of 25 μg/mL in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and kinetic binding experiments were performed at 25° C. using 1×HBS-EP+buffer. Test and control antibodies were injected over the anti-Fc surface at concentrations of 5-10 μg/mL for 12 seconds at a flow rate of 10 μL/min on flow cells 2, 3 and 4, followed by a buffer wash for 30 seconds at the same flow rate. Kinetic characterization of antibody samples was carried out with a single concentration of antigen (for off-rate ranking) or a 1:2 dilution series of antigen (for kinetic characterization) and 1 injection of 0 nM antigen. After capturing ligand (antibody) on the anti-Fc surface, the analyte (human FOLR1-HIS) was bound at 50, 25, 12.5, 6.25 and 0 nM for 180 seconds, followed by a 600 second dissociation phase at a flow rate of 50 μL/min. Between each ligand capture and analyte binding cycle, regeneration was carried out using 2 injections of 10 mM glycine pH 2.0 for 30 seconds at 30 L/min, followed by a 30 second buffer wash step.

The data were fit with the Biacore T200 Evaluation software, using a 1:1 Langmuir binding model. K_{D} (affinity, nM) was determined as a ratio of the kinetic rate constants calculated from the fits of the association and dissociation phases.

### Example 4

### Flow Cytometry-Based Cell Binding Assay

Variants with expression levels >250 nM were tested in a fluorescence-activated cell sorting (FACS) cell-binding assay. CHO cells were transfected to stably express human (CHO-hFOLR1), cynomolgus (CHO-cFOLR1), or mouse (CHO-mFOLR1) target molecule FOLR1 on the cell surface. Parental CHO cells were used as a negative control to determine background binding levels. Parental CHO and stably transfected CHO-hFOLR1, CHO-cFOLR1, and CHO-mFOLR1 cells were cultured in Ham's F-12: high glucose DMEM (50:50) (Corning, Cellgro-Mediatech) supplemented with 10% heat-inactivated fetal bovine serum (Corning, Cellgro-Mediatech), 1% Penicillin/Streptomycin (Corning, Cellgro-Mediatech) and 2 mmol/L-glutamax (Life Technology).

A mixture of fluoresecent-labeled parental CHO cells and unlabeled CHO-hFOLRlcells were prepared as follows. Parental CHO cells were washed twice in PBS and incubated in PBS containing with 1 nM CellTrace™ Oregon Green488® (Life Technologies) at 37° C. for 30 minutes. Labeled parental CHO cells were then washed 2× with Ham's F-12 media and 2× with FACS buffer (PBS with 1% bovine serum albumin). Unlabeled CHO-hFOLR1 cells were similarly washed and prepared. Labeled parental CHO and unlabeled CHO-hFOLR1 cells were combined at 1:1 ratio and seeded at 50 μL per well (200,000 cells per well) in 96 well polypropylene plates. Cells were mixed with 50 μL of test antibodies (i.e., anti-FOLR1 variants) serially diluted in FACS buffer and incubated on ice for 60 mins. Cells were washed with FACS buffer and incubated on ice for 60 mins with 100 μL FACS buffer containing 2.5 μg/mL R-Phycoerythrin-conjugated goat anti-Human IgG (Jackson ImmunoResearch Laboratories, West Grove, Pa.). Cells were washed twice with FACS buffer, fixed in 2% paraformaldehyde in PBS (Santa Cruz Biotechnology; Dallas, Tex.) for 10 mins on ice in the dark, and analyzed using the BD LSR II Flow Cytometer (BD Biosciences; San Jose, Calif.). Data were analyzed using FlowJo® software (FlowJo, LLC; Ashland, Oreg.) to determine mean fluorescence intensities. Binding constants were calculated using the statistical software, GraphPad Prism (GraphPad Software; La Jolla, Calif.) using the nonlinear regression equation, one site-specific binding with Hill slope. Secondary antibody alone was used as a control, in addition to measuring non-specific antibody binding to CHO parental cells.

This procedure was repeated to assess cell binding in CHO-cFOLR1 and CHO-mFOLR1 cells.

### Example 5

### Cell-Killing Analysis

The internalization of the antibodies was evaluated by a secondary antibody cell killing assay on target positive cells. FOLR1-positive KB cells were obtained from ATCC, and FOLR1-positive Igrovl cells were obtained from NIH. The cells were maintained in Ham's F-12: high glucose DMEM (50:50) (Corning, Cellgro-Mediatech) supplemented with 10% heat-inactivated fetal bovine serum (Corning, Cellgro-Mediatech, Manassas, Va.), 1% Penicillin/Streptomycin (Corning, Cellgro-Mediatech, Manassas, Va.) and 2 mmol/L-glutamax (Thermo Fisher Scientific, Waltham, Mass.). Adherent cells were washed twice with calcium and magnesium-free Hanks Balanced Salt Solution (HBSS), harvested with HYQ®TASE™ (Hyclone; Thermo Fisher Scientific, Waltham, Mass.) and counted by the Vi-CELL Cell Viability Analyzers (Beckman Coulter, Indianapolis, Ind.). A total of 625 cells were seeded in each well of a 384-well flat bottom white polystyrene plate. Lead antibodies were formulated at 4-fold starting concentration in the cell culture medium and filtered through MultiScreenHTS 96-Well Filter Plates (Millipore; Billerica, Mass.). Serial dilutions of test antibody (1:3 serial dilution starting from 200 nM) was added into treatment wells, and an anti-human Fc nanobody conjugated to hemiasterlin via a cleavable linker was then added into each well at a fixed final concentration of 20 nM. Assay plates were cultured at 37° C. in a CO₂ incubator for 120 hrs before assay. For cell viability measurement, 30 μL of Cell Titer-Glo® reagent (Promega Corp. Madison, Wis.) was added into each well, and plates were processed as per product instructions. Relative luminescence was measured on an ENVISION® plate reader (Perkin-Elmer; Waltham, Mass.). Relative luminescence readings were converted to percent viability using untreated cells as controls. Data was fitted with nonlinear regression analysis, using a log(inhibitor) vs. response-variable slope, 4 parameter fit with GraphPad Prism (GraphPad v 5.0, Software; San Diego, Calif.). Data was expressed as relative cell viability (ATP content) % vs. dose of antibody.

### Example 6

### Generation of Hybridoma

Immunocompetent mice (C57BL/6) were immunized with mouse MC38 cells overexpressing human FOLR1. FOLR1-specific antibodies were detected in the sera, and the spleen was harvested and fused with P3X cells to generate the hybridomas (Aragen Biosciences, Morgan Hill, Calif.), similar to what has been previously described. See Chronopoulou, et al., 2014, Methods Mol Biol 1131:47-70, and Kim, et al., 2014, Methods Mol Biol 1131:31-45, each of which is incorporated herein by reference in its entirety. Total RNA was extracted from hybridoma cells using QIAGEN RNeasy Mini Kit (Cat No. 74104) and converted to cDNA using a Clontech SMARTer RACE cDNA Amplification Kit (Cat. No. 634923) (Lake Pharma, Belmont, Calif.). Positive clones were identified by gel electrophoresis, cloned using an Invitrogen TOPO kit, and sequenced using standard Sanger methods. The CDRs for m6D1 were grafted onto human antibody frameworks V_{H}1-18, V_{H}3-33, V_{H}2-5, V_{H}2-70, V_{H}4-30-4, Vk1-5, Vk3-11, Vk2-30, Vk1-33, and Vk1-16 by standard methodology to yield humanized antibodies. See Kuramochi, et al., 2014, Methods Mol Biol 1060:123-137, which is incorporated herein by reference in its entirety. Of these grafts, the h6D1-HC3/LC4 (V_{H}3-33/Vk3-11 grafts) and h6D1-HC3/LC5 (V_{H}3-33/Vk1-5 grafts) IgGs gave the best yield when expressed in cell-free and maintained the highest affinity. Both HC3/LC4 and HC3/LC5 humanized variants were progressed into affinity maturation by Fab-based ribosome display (as described above) targeting the heavy chain CDRs by soft-randomization leaving the light-chain constant, as described in Stafford, et al., 2014, Protein Eng Des Sel 4:97-109, which is incorporated herein by reference in its entirety.

Certain antibodies were generated by affinity maturation of humanized mouse antibodies. Exemplary antibody candidates are reported in Table 6, below.

TABLE 6
Affinity-matured humanized antibodies (SRP2060).

| | | SEQ ID | | SEQ ID |
|---|---|---|---|---|
| Antibody | VH | NO. | VL | NO. |
| 56 | SRP2060-E10 | 363 | H6D1-LC4 | 368 |
| 57 | SRP2060-E05 | 364 | H6D1-LC4 | 368 |
| 58 | SRP2060-B01 | 365 | H6D1-LC5 | 369 |
| 59 | SRP2060-A06 | 366 | H6D1-LC5 | 369 |

### Example 7

### Characteristics of Illustrative Anti-FOLR1 Antibodies

Tables 7 through 9 show results obtained using the illustrative antibodies described herein.

Table 7 shows results obtained with antibodies isolated from affinity-maturation of initial antibody leads obtained from a naïve Fab TRiM ribosome display library, constructed on a Trastuzumab heavy chain (HC) framework.

Table 8 shows kinetic binding results obtained for the same antibodies listed in Table 7.

Table 9 shows results obtained from antibodies isolated from humanized mouse clone candidates.

TABLE 7
Affinity-matured antibodies from initial leads (Trastuzumab HC framework).

| | KB, 2° | | Igrov, 2° | | | | | | | |
|---|---|---|---|---|---|---|---|---|---|---|
| | Antibody | | Antibody | | | | | | | |
| | Cell | | Cell | | CHO- | | | | | |
| | Killing, | | Killing, | | human | | CHO-cyno | | CHO-mouse | |
| | Nb-239 | | Nb-SC239 | | FoIR1 | | FoIR1 | | FoIR1 | |
| | EC50 | Span | EC50 | Span | Bmax | K_{D} | Bmax | K_{D} | Bmax | K_{D} |
| Fab-HC Variant ID | (nM) | (%) | (nM) | (%) | (MFI) | (nM) | (MFI) | (nM) | (MFI) | (nM) |
| SRP1848-A01 | 0.064 | 94 | 0.015 | 71 | 25899 | 0.39 | 22552 | 0.44 | 16475 | 0.8 |
| SRP1848-A02 | 0.028 | 94 | 0.039 | 71 | 24710 | 0.64 | 18500 | 0.50 | 18569 | 2.4 |
| SRP1848-A07 | 0.062 | 95 | 0.029 | 68 | 29182 | 0.61 | 23643 | 0.43 | 9646 | 0.9 |
| SRP1848-C03 | 0.074 | 93 | 0.035 | 72 | 29143 | 0.51 | 25148 | 0.50 | 3310 | 3.0 |
| SRP1848-F04 | 0.096 | 93 | 0.015 | 73 | 26867 | 0.73 | 26353 | 0.55 | 2741 | 11.0 |
| SRP1848-B04 | 0.035 | 94 | 0.018 | 70 | 27818 | 0.72 | 27796 | 0.65 | 2187 | 17.9 |
| SRP1848-B11 | 0.058 | 93 | 0.026 | 74 | 28394 | 0.56 | 22885 | 0.34 | 1632 | 3.9 |
| SRP1848-F07 | 0.057 | 92 | 0.018 | 71 | 27371 | 0.58 | 18662 | 0.56 | 1387 | 8.8 |
| SRP1848-E06 | 0.060 | 93 | 0.025 | 74 | 25611 | 0.48 | 15755 | 0.26 | 2349 | 1.2 |
| SRP1848-A09 | 0.060 | 93 | 0.026 | 71 | 28910 | 0.61 | 20248 | 0.31 | 7990 | 1.0 |
| SRP1848-E07 | 0.059 | 94 | 0.013 | 73 | 27284 | 0.54 | 20381 | 0.23 | 11837 | 1.2 |
| SRP1848-G03 | 0.064 | 91 | 0.021 | 76 | 26424 | 0.82 | 19238 | 0.44 | 2220 | 2.4 |
| SRP1848-A04 | 0.052 | 92 | 0.015 | 64 | 26810 | 0.43 | 23055 | 0.30 | 3888 | 2.0 |
| SRP1848-H01 | 0.049 | 96 | 0.016 | 67 | 26985 | 0.59 | 17227 | 0.28 | 3950 | 33.8 |
| SRP1848-B10 | 0.040 | 97 | 0.020 | 71 | 28186 | 0.83 | 21268 | 0.44 | 2455 | 7.3 |
| SRP1848-C07 | 0.065 | 93 | 0.013 | 67 | 28757 | 0.62 | 18136 | 0.23 | 3170 | 1.4 |
| SRP1848-F05 | 0.061 | 94 | 0.015 | 74 | 27155 | 0.72 | 24731 | 0.61 | 5100 | 18.0 |
| SRP1848-D02 | 0.034 | 93 | 0.027 | 71 | 28804 | 0.60 | 27973 | 0.61 | 916 | 87.0 |
| SRP1848-A08 | 0.039 | 93 | 0.013 | 65 | 28554 | 0.62 | 26197 | 0.45 | 3202 | 2.5 |
| SRP1848-E03 | 0.057 | 94 | 0.027 | 73 | 26694 | 0.76 | 17427 | 0.43 | 5939 | 0.5 |
| SRP1848-A10 | 0.033 | 96 | 0.027 | 75 | 27097 | 0.66 | 14816 | 0.47 | 10167 | 1.2 |
| SRP1848-F10 | 0.038 | 94 | 0.009 | 68 | 25554 | 0.36 | 20700 | 0.40 | 1742 | 6.9 |
| SRP1848-D05 | 0.055 | 92 | 0.030 | 73 | 26748 | 0.57 | 22202 | 0.45 | 1360 | 14.0 |
| SRP1848-C01 | 0.060 | 90 | 0.023 | 68 | 28527 | 0.66 | 25941 | 0.60 | 1369 | 26.0 |
| SRP1848-F01 | 0.047 | 91 | 0.018 | 69 | 25240 | 0.56 | 21491 | 0.43 | 3750 | 1.8 |
| SRP1848-D04 | 0.380 | 97 | 0.068 | 77 | 29297 | 2.21 | 25737 | 2.84 | NB | NB |
| SRP1848-E05 | 0.071 | 95 | 0.027 | 78 | 27306 | 0.46 | 28170 | 0.55 | NB | NB |
| SRP1848-A06 | 0.046 | 93 | 0.020 | 72 | 24521 | 0.47 | 20170 | 0.30 | 2767 | 2.4 |
| SRP1848-B01 | 0.064 | 95 | 0.031 | 82 | 26634 | 1.06 | 23881 | 0.83 | 3404 | 16.4 |
| SRP1848-C04 | 0.006 | 94 | 0.016 | 68 | 26269 | 0.44 | 22014 | 0.86 | 2506 | 62.0 |
| SRP1848-C10 | 0.057 | 96 | 0.036 | 75 | 27465 | 0.91 | 15966 | 0.27 | 2326 | 5.6 |
| SRP1848-B09 | 0.073 | 97 | 0.027 | 74 | 25152 | 0.46 | 25213 | 0.99 | 1424 | 78.0 |
| SRP1848-C05 | 0.073 | 92 | 0.021 | 62 | 26836 | 0.52 | 15199 | 0.35 | 4134 | 4.8 |
| SRP1848-F02 | 0.054 | 92 | 0.009 | 54 | 25714 | 0.62 | 14911 | 0.19 | 2741 | 2.6 |
| SRP1848-F08 | 0.061 | 94 | 0.024 | 77 | 26483 | 0.91 | 21024 | 1.07 | NB | NB |
| SRP1848-D07 | 0.075 | 94 | 0.032 | 71 | 25738 | 0.77 | 24272 | 0.92 | NB | NB |
| SRP1848-F11 | 0.054 | 91 | 0.017 | 70 | 26774 | 0.75 | 21790 | 0.47 | 1762 | 4.6 |
| SRP1848-F09 | 0.056 | 93 | 0.050 | 79 | 23816 | 0.36 | 24178 | 0.75 | 1671 | 90.7 |
| SRP1848-D10 | 0.016 | 90 | 0.012 | 54 | 26468 | 0.48 | 20578 | 0.52 | 1859 | 13.0 |
| SRP1848-G01 | 0.070 | 91 | 0.022 | 66 | 27406 | 0.98 | 20913 | 0.56 | 1993 | 4.6 |
| SRP1848-B06 | 0.058 | 95 | 0.022 | 72 | 25070 | 0.67 | 26767 | 1.21 | NB | NB |
| SRP1848-D03 | 0.160 | 98 | 0.038 | 76 | 25977 | 1.90 | 14130 | 0.58 | 3170 | 9.5 |
| SRP1848-B07 | 0.079 | 96 | 0.038 | 73 | 25612 | 0.66 | 25491 | 1.05 | NB | NB |
| SRP1848-E02 | 0.046 | 93 | 0.025 | 71 | 23847 | 0.53 | 18717 | 0.59 | 1473 | 21.0 |
| SRP1848-B03 | 0.050 | 94 | 0.028 | 66 | 26338 | 0.82 | 17228 | 0.41 | 2722 | 6.4 |
| SRP1848-E01 | 0.088 | 92 | 0.029 | 72 | 26430 | 1.01 | 22420 | 0.96 | NB | NB |
| SRP1848-B05 | 0.065 | 94 | 0.040 | 72 | 24536 | 0.65 | 21871 | 0.64 | NB | NB |
| SRP1848-D09 | 0.042 | 91 | 0.023 | 70 | 24966 | 0.46 | 21306 | 0.65 | NB | NB |
| SRP1848-F06 | 0.066 | 94 | 0.032 | 77 | 25598 | 0.87 | 26528 | 0.86 | NB | NB |
| SRP1848-G10 | 0.046 | 97 | 0.019 | 79 | 25269 | 0.49 | 14163 | 0.24 | 2891 | 4.3 |
| SRP1848-G04 | 0.051 | 92 | 0.016 | 75 | 25156 | 0.76 | 12538 | 0.25 | 1999 | 2.5 |
| SRP1848-G06 | 0.057 | 96 | 0.026 | 81 | 25838 | 0.63 | 12830 | 0.31 | 1857 | 11.1 |
| SRP1848-G07 | 0.058 | 94 | 0.038 | 78 | 24939 | 0.78 | 13668 | 0.35 | 1978 | 2.9 |
| SRP1848-G09 | 0.073 | 97 | 0.036 | 83 | 25066 | 0.59 | 17685 | 0.35 | 2184 | 6.4 |
| SRP1848-G11 | 0.040 | 97 | 0.023 | 84 | 27191 | 0.68 | 11837 | 0.26 | 2744 | 7.6 |

TABLE 8
Affinity-matured antibodies from initial leads (Trastuzumab HC
framework): Kinetic binding results

| | Biacore Kinetics | | |
|---|---|---|---|
| Variant ID | ka (1/Ms) | kd (1/s) | K_{D} (M) |
| SRP1848-A01 | 8.29E+05 | 1.55E−03 | 1.87E−09 |
| SRP1848-A02 | 5.25E+05 | 8.82E−03 | 1.68E−08 |
| SRP1848-A07 | 1.01E+06 | 8.66E−04 | 8.55E−10 |
| SRP1848-C03 | 1.36E+06 | 1.52E−03 | 1.11E−09 |
| SRP1848-F04 | 8.15E+05 | 1.08E−03 | 1.32E−09 |
| SRP1848-B04 | 7.80E+05 | 1.17E−03 | 1.50E−09 |
| SRP1848-B11 | 1.22E+06 | 1.86E−03 | 1.52E−09 |
| SRP1848-F07 | 1.60E+06 | 2.49E−03 | 1.56E−09 |
| SRP1848-E06 | 9.44E+05 | 1.54E−03 | 1.63E−09 |
| SRP1848-A09 | 7.30E+05 | 1.33E−03 | 1.82E−09 |
| SRP1848-E07 | 1.25E+06 | 2.40E−03 | 1.91E−09 |
| SRP1848-G03 | 9.90E+05 | 1.97E−03 | 1.99E−09 |
| SRP1848-A04 | 1.61E+06 | 3.26E−03 | 2.03E−09 |
| SRP1848-H01 | 6.59E+05 | 1.39E−03 | 2.11E−09 |
| SRP1848-B10 | 6.81E+05 | 1.48E−03 | 2.18E−09 |
| SRP1848-C07 | 8.56E+05 | 1.89E−03 | 2.21E−09 |
| SRP1848-F05 | 6.56E+05 | 1.57E−03 | 2.40E−09 |
| SRP1848-D02 | 8.51E+05 | 2.05E−03 | 2.41E−09 |
| SRP1848-A08 | 4.93E+05 | 1.19E−03 | 2.42E−09 |
| SRP1848-E03 | 6.88E+05 | 1.83E−03 | 2.67E−09 |
| SRP1848-A10 | 1.20E+06 | 3.30E−03 | 2.74E−09 |
| SRP1848-F10 | 8.72E+05 | 2.47E−03 | 2.83E−09 |
| SRP1848-D05 | 6.75E+05 | 1.98E−03 | 2.93E−09 |
| SRP1848-C01 | 7.30E+05 | 2.23E−03 | 3.05E−09 |
| SRP1848-F01 | 1.14E+06 | 3.62E−03 | 3.18E−09 |
| SRP1848-D04 | 4.97E+05 | 1.73E−03 | 3.48E−09 |
| SRP1848-E05 | 7.16E+05 | 2.51E−03 | 3.51E−09 |
| SRP1848-A06 | 1.37E+06 | 4.83E−03 | 3.51E−09 |
| SRP1848-B01 | 1.13E+06 | 4.16E−03 | 3.67E−09 |
| SRP1848-C04 | 1.29E+06 | 4.99E−03 | 3.86E−09 |
| SRP1848-C10 | 8.99E+05 | 3.63E−03 | 4.03E−09 |
| SRP1848-B09 | 1.55E+06 | 6.61E−03 | 4.26E−09 |
| SRP1848-C05 | 1.06E+06 | 4.54E−03 | 4.29E−09 |
| SRP1848-F02 | 1.42E+06 | 6.37E−03 | 4.49E−09 |
| SRP1848-F08 | 5.94E+05 | 2.72E−03 | 4.58E−09 |
| SRP1848-D07 | 1.09E+06 | 5.11E−03 | 4.70E−09 |
| SRP1848-F11 | 8.28E+05 | 3.90E−03 | 4.71E−09 |
| SRP1848-F09 | 1.40E+06 | 6.79E−03 | 4.85E−09 |
| SRP1848-D10 | 1.13E+06 | 5.58E−03 | 4.95E−09 |
| SRP1848-G01 | 4.44E+05 | 2.26E−03 | 5.09E−09 |
| SRP1848-B06 | 6.20E+05 | 3.17E−03 | 5.10E−09 |
| SRP1848-D03 | 1.03E+06 | 5.35E−03 | 5.19E−09 |
| SRP1848-B07 | 7.06E+05 | 3.78E−03 | 5.35E−09 |
| SRP1848-E02 | 1.14E+06 | 7.07E−03 | 6.21E−09 |
| SRP1848-B03 | 1.13E+06 | 8.59E−03 | 7.63E−09 |
| SRP1848-E01 | 6.64E+05 | 5.22E−03 | 7.87E−09 |
| SRP1848-B05 | 9.76E+05 | 8.85E−03 | 9.07E−09 |
| SRP1848-D09 | 1.07E+06 | 1.08E−02 | 1.01E−08 |
| SRP1848-F06 | 4.56E+05 | 7.75E−03 | 1.70E−08 |
| SRP1848-G10 | 7.58E+05 | 3.45E−03 | 4.55E−09 |
| SRP1848-G04 | 5.91E+05 | 3.79E−03 | 6.40E−09 |
| SRP1848-G06 | 5.69E+05 | 3.81E−03 | 6.70E−09 |
| SRP1848-G07 | 6.05E+05 | 4.51E−03 | 7.45E−09 |
| SRP1848-G09 | 8.56E+05 | 6.46E−03 | 7.56E−09 |
| SRP1848-G11 | 6.96E+05 | 6.37E−03 | 9.14E−09 |

TABLE 9
Results obtained with humanized 6D1 (2060) antibodies.

| | | | | Igrov 2° Antibody | |
|---|---|---|---|---|---|
| | | | | Cell Killing, | |
| | Biacore kinetics | | | Nb-SC239 | |
| | | kd | | EC50 | |
| SRP | ka (1/Ms) | (1/s) | KD (M) | (nM) | span (%) |
| SRP2060-E10 | 5.82E+05 | 1.20E−03 | 2.06E−09 | 0.061 | 68 |
| SRP2060-E05 | 5.41E+05 | 1.58E−03 | 2.92E−09 | 0.22 | 71 |
| SRP2060-B01 | 5.61E+05 | 1.47E−03 | 2.62E−09 | 0.045 | 76 |
| SRP2060-A06 | 5.47E+−5 | 7.29E−03 | 1.33E−08 | 0.013 | 66 |

### Example 8

### Sequences

Table 10 provides sequences referred to herein.

TABLE 10
Sequences

| SEQ | | | | |
|---|---|---|---|---|
| ID | | | | |
| NO: | Molecule | Region | Scheme | Sequence |
| 1 | Human folate | | | MAQRMTTQLLLLLVWVAVVGEAQTRIAW |
| | receptor alpha | | | ARTELLNVCMNAKHHKEKPGPEDKLHEQ |
| | (hFOLR1) | | | CRPWRKNACCSTNTSQEAHKDVSYLYRF |
| | | | | NWNHCGEMAPACKRHFIQDTCLYECSPN |
| | | | | LGPWIQQVDQSWRKERVLNVPLCKEDCE |
| | | | | QWWEDCRTSYTCKSNWHKGWNWTSGFNK |
| | | | | CAVGAACQPFHEYEPTPTVLCNEIWTHS |
| | | | | YKVSNYSRGSGRCIQMWFDPAQGNPNEE |
| | | | | VARFYAAAMSGAGPWAAWPFLLSLALML |
| | | | | LWLLS |
| 2 | Cynomolgus | | | MAQRMTTQLLLLLVWVAVVGEAQTRTAR |
| | folate | | | ARTELLNVCMNAKHHKEKPGPEDKLHEQ |
| | receptor alpha | | | CRPWKKNACCSTNTSQEAHKDVSYLYRF |
| | | | | NWNHCGEMAPACKRHFIQDTCLYECSPN |
| | | | | LGPWIQQVDQSWRKERVLNVPLCKEDCE |
| | | | | RWWEDCRTSYTCKSNWHKGWNWTSGFNK |
| | | | | CPVGAACQPFHEYEPTPTVLCNEIWTYS |
| | | | | YKVSNYSRGSGRCIQMWFDPAQGNPNEE |
| | | | | VARFYAAAMSGAGPWAAWPLLLSLALTL |
| | | | | LWLLS |
| 3 | Murine folate | | | MAHLMTVQLLLLVMWMAECAQSRATRAR |
| | receptor alpha | | | TELLNVCMDAKHHKEKPGPEDNLHDQCS |
| | | | | PWKTNSCCSTNTSQEAHKDISYLYRFNW |
| | | | | NHCGTMTSECKRHFIQDTCLYECSPNLG |
| | | | | PWIQQVDQSWRKERILDVPLCKEDCQQW |
| | | | | WEDCQSSFTCKSNWHKGWNWSSGHNECP |
| | | | | VGASCHPFTFYFPTSAALCEEIWSHSYK |
| | | | | LSNYSRGSGRCIQMWFDPAQGNPNEEVA |
| | | | | RFYAEAMSGAGFHGTWPLLCSLSLVLLW |
| | | | | VIS |
| 4 | SRP1848-A01 | CDR-H1 | Chothia | GFNITRY |
| 5 | SRP1848-A02 | CDR-H1 | Chothia | GFNISGF |
| 6 | SRP1848-A04 | CDR-H1 | Chothia | GFNIDQS |
| 7 | SRP1848-A06 | CDR-H1 | Chothia | GFNIGNS |
| 8 | SRP1848-A07 | CDR-H1 | Chothia | GFNIGYH |
| 9 | SRP1848-A08 | CDR-H1 | Chothia | GSNIRKH |
| 10 | SRP1848-A09 | CDR-H1 | Chothia | GENIRKQ |
| 11 | SRP1848-A10 | CDR-H1 | Chothia | GENIRKY |
| 12 | SRP1848-B01 | CDR-H1 | Chothia | GFNIRNY |
| 13 | SRP1848-B03 | CDR-H1 | Chothia | GFNISMK |
| 14 | SRP1848-B04 | CDR-H1 | Chothia | SFNISNH |
| 15 | SRP1848-B05 | CDR-H1 | Chothia | GFNISNY |
| 16 | SRP1848-B06 | CDR-H1 | Chothia | GFNISNY |
| 17 | SRP1848-B07 | CDR-H1 | Chothia | GFNISRF |
| 18 | SRP1848-B09 | CDR-H1 | Chothia | GFNITNY |
| 19 | SRP1848-B10 | CDR-H1 | Chothia | GFNTTTK |
| 20 | SRP1848-B11 | CDR-H1 | Chothia | GFNIGNN |
| 21 | SRP1848-001 | CDR-H1 | Chothia | GFNIGNS |
| 22 | SRP1848-0O3 | CDR-H1 | Chothia | GFNIGVY |
| 23 | SRP1848-004 | CDR-H1 | Chothia | GFNIRHY |
| 24 | SRP1848-005 | CDR-H1 | Chothia | GFNIRKY |
| 25 | SRP1848-007 | CDR-H1 | Chothia | GFNIRKY |
| 26 | SRP1848-C10 | CDR-H1 | Chothia | GFNIRTY |
| 27 | SRP1848-D02 | CDR-H1 | Chothia | GFNISHN |
| 28 | SRP1848-D03 | CDR-H1 | Chothia | GFNIRYF |
| 29 | SRP1848-D04 | CDR-H1 | Chothia | GFNISHY |
| 30 | SRP1848-D05 | CDR-H1 | Chothia | GFNISIS |
| 31 | SRP1848-D07 | CDR-H1 | Chothia | GFNISKY |
| 32 | SRP1848-D09 | CDR-H1 | Chothia | GFNISNY |
| 33 | SRP1848-D10 | CDR-H1 | Chothia | GFNISRN |
| 34 | SRP1848-E01 | CDR-H1 | Chothia | GFNITNK |
| 35 | SRP1848-E02 | CDR-H1 | Chothia | GFNIGKY |
| 36 | SRP1848-E03 | CDR-H1 | Chothia | GFNIGNY |
| 37 | SRP1848-E05 | CDR-H1 | Chothia | GFNIGVY |
| 38 | SRP1848-E06 | CDR-H1 | Chothia | GFNINRY |
| 39 | SRP1848-E07 | CDR-H1 | Chothia | GFNIRKS |
| 40 | SRP1848-F01 | CDR-H1 | Chothia | GFNIRTY |
| 41 | SRP1848-F02 | CDR-H1 | Chothia | GFNIRTY |
| 42 | SRP1848-F04 | CDR-H1 | Chothia | GFNISNY |
| 43 | SRP1848-F05 | CDR-H1 | Chothia | GFNISKS |
| 44 | SRP1848-F06 | CDR-H1 | Chothia | GFNISLS |
| 45 | SRP1848-F07 | CDR-H1 | Chothia | GFNISNH |
| 46 | SRP1848-F08 | CDR-H1 | Chothia | GFNISNH |
| 47 | SRP1848-F09 | CDR-H1 | Chothia | GFNISNH |
| 48 | SRP1848-F10 | CDR-H1 | Chothia | GFNISNN |
| 49 | SRP1848-F11 | CDR-H1 | Chothia | GFNISNN |
| 50 | SRP1848-G01 | CDR-H1 | Chothia | GFNISRH |
| 51 | SRP1848-G03 | CDR-H1 | Chothia | GFNISTY |
| 52 | SRP1848-G04 | CDR-H1 | Chothia | GFNIHST |
| 53 | SRP1848-G06 | CDR-H1 | Chothia | GFNIRST |
| 54 | SRP1848-G07 | CDR-H1 | Chothia | GFNIHST |
| 55 | SRP1848-G09 | CDR-H1 | Chothia | GFNIRGT |
| 56 | SRP1848-G10 | CDR-H1 | Chothia | GFNIRST |
| 57 | SRP1848-G11 | CDR-H1 | Chothia | GFNISST |
| 58 | SRP1848-H01 | CDR-H1 | Chothia | GFNIRTQ |
| 59 | SRP2060-E10 | CDR-H1 | Chothia | GFSLSTFGM |
| 60 | SRP2060-E05 | CDR-H1 | Chothia | GFSLSTFGM |
| 61 | SRP2060-B01 | CDR-H1 | Chothia | GFSLSTFGM |
| 62 | SRP2060-A06 | CDR-H1 | Chothia | GFSLSTFGM |
| 63 | SRP1848-A01 | CDR-H1 | Kabat | RYSIH |
| 64 | SRP1848-A02 | CDR-H1 | Kabat | GFRIH |
| 65 | SRP1848-A04 | CDR-H1 | Kabat | QSSIH |
| 66 | SRP1848-A06 | CDR-H1 | Kabat | NSYIH |
| 67 | SRP1848-A07 | CDR-H1 | Kabat | YHSIH |
| 68 | SRP1848-A08 | CDR-H1 | Kabat | KHSIH |
| 69 | SRP1848-A09 | CDR-H1 | Kabat | KQSIH |
| 70 | SRP1848-A10 | CDR-H1 | Kabat | KYSIH |
| 71 | SRP1848-B01 | CDR-H1 | Kabat | NYSIH |
| 72 | SRP1848-B03 | CDR-H1 | Kabat | MKYIH |
| 73 | SRP1848-B04 | CDR-H1 | Kabat | NHSIH |
| 74 | SRP1848-B05 | CDR-H1 | Kabat | NYYIH |
| 75 | SRP1848-B06 | CDR-H1 | Kabat | NYYIH |
| 76 | SRP1848-B07 | CDR-H1 | Kabat | RFYIH |
| 77 | SRP1848-B09 | CDR-H1 | Kabat | NYYIH |
| 78 | SRP1848-B10 | CDR-H1 | Kabat | TKSIH |
| 79 | SRP1848-B11 | CDR-H1 | Kabat | NNSIH |
| 80 | SRP1848-001 | CDR-H1 | Kabat | NSYIH |
| 81 | SRP1848-0O3 | CDR-H1 | Kabat | VYSIH |
| 82 | SRP1848-004 | CDR-H1 | Kabat | HYSIH |
| 83 | SRP1848-005 | CDR-H1 | Kabat | KYSIH |
| 84 | SRP1848-007 | CDR-H1 | Kabat | KYSIH |
| 85 | SRP1848-C10 | CDR-H1 | Kabat | TYYIH |
| 86 | SRP1848-D02 | CDR-H1 | Kabat | HNYIH |
| 87 | SRP1848-D03 | CDR-H1 | Kabat | YFSIH |
| 88 | SRP1848-D04 | CDR-H1 | Kabat | HYSIH |
| 89 | SRP1848-D05 | CDR-H1 | Kabat | ISYIH |
| 90 | SRP1848-D07 | CDR-H1 | Kabat | KYYIH |
| 91 | SRP1848-D09 | CDR-H1 | Kabat | NYYIH |
| 92 | SRP1848-D10 | CDR-H1 | Kabat | RNSIH |
| 93 | SRP1848-E01 | CDR-H1 | Kabat | NKYIH |
| 94 | SRP1848-E02 | CDR-H1 | Kabat | KYSIH |
| 95 | SRP1848-E03 | CDR-H1 | Kabat | NYYIH |
| 96 | SRP1848-E05 | CDR-H1 | Kabat | VYYIH |
| 97 | SRP1848-E06 | CDR-H1 | Kabat | RYYIH |
| 98 | SRP1848-E07 | CDR-H1 | Kabat | KSSIH |
| 99 | SRP1848-F01 | CDR-H1 | Kabat | TYSIH |
| 100 | SRP1848-F02 | CDR-H1 | Kabat | TYSIH |
| 101 | SRP1848-F04 | CDR-H1 | Kabat | NYSIH |
| 102 | SRP1848-F05 | CDR-H1 | Kabat | KSSIH |
| 103 | SRP1848-F06 | CDR-H1 | Kabat | LSYIH |
| 104 | SRP1848-F07 | CDR-H1 | Kabat | NHSIH |
| 105 | SRP1848-F08 | CDR-H1 | Kabat | NHSIH |
| 106 | SRP1848-F09 | CDR-H1 | Kabat | NHYIH |
| 107 | SRP1848-F10 | CDR-H1 | Kabat | NNSIH |
| 108 | SRP1848-F11 | CDR-H1 | Kabat | NNYIH |
| 109 | SRP1848-G01 | CDR-H1 | Kabat | RHSIH |
| 110 | SRP1848-G03 | CDR-H1 | Kabat | TYYIH |
| 111 | SRP1848-G04 | CDR-H1 | Kabat | STDIH |
| 112 | SRP1848-G06 | CDR-H1 | Kabat | STDIH |
| 113 | SRP1848-G07 | CDR-H1 | Kabat | STDIH |
| 114 | SRP1848-G09 | CDR-H1 | Kabat | GTDIH |
| 115 | SRP1848-G10 | CDR-H1 | Kabat | STDIH |
| 116 | SRP1848-G11 | CDR-H1 | Kabat | STDIH |
| 117 | SRP1848-H01 | CDR-H1 | Kabat | TQSIH |
| 118 | SRP2060-E10 | CDR-H1 | Kabat | TFGMGVG |
| 119 | SRP2060-E05 | CDR-H1 | Kabat | TFGMGVG |
| 120 | SRP2060-B01 | CDR-H1 | Kabat | TFGMGVG |
| 121 | SRP2060-A06 | CDR-H1 | Kabat | TFGMGVG |
| 122 | SRP1848-A01 | CDR-H2 | Chothia | LPESGG |
| 123 | SRP1848-A02 | CDR-H2 | Chothia | YPESGA |
| 124 | SRP1848-A04 | CDR-H2 | Chothia | YPVDGT |
| 125 | SRP1848-A06 | CDR-H2 | Chothia | TPIDGN |
| 126 | SRP1848-A07 | CDR-H2 | Chothia | FPVDGT |
| 127 | SRP1848-A08 | CDR-H2 | Chothia | YPNDGT |
| 128 | SRP1848-A09 | CDR-H2 | Chothia | FPNDGT |
| 129 | SRP1848-A10 | CDR-H2 | Chothia | FPIDDI |
| 130 | SRP1848-B01 | CDR-H2 | Chothia | YPVDGI |
| 131 | SRP1848-B03 | CDR-H2 | Chothia | TPIDGM |
| 132 | SRP1848-B04 | CDR-H2 | Chothia | YPVDGI |
| 133 | SRP1848-B05 | CDR-H2 | Chothia | SPIDGY |
| 134 | SRP1848-B06 | CDR-H2 | Chothia | TPIDGY |
| 135 | SRP1848-B07 | CDR-H2 | Chothia | SPYDGF |
| 136 | SRP1848-B09 | CDR-H2 | Chothia | TPVDGY |
| 137 | SRP1848-B10 | CDR-H2 | Chothia | YPRDGI |
| 138 | SRP1848-B11 | CDR-H2 | Chothia | SPIDGF |
| 139 | SRP1848-001 | CDR-H2 | Chothia | TPNDGY |
| 140 | SRP1848-0O3 | CDR-H2 | Chothia | YPIDGN |
| 141 | SRP1848-004 | CDR-H2 | Chothia | YPGPGN |
| 142 | SRP1848-005 | CDR-H2 | Chothia | FPIDGI |
| 143 | SRP1848-007 | CDR-H2 | Chothia | FPIDGI |
| 144 | SRP1848-C10 | CDR-H2 | Chothia | SPIDGY |
| 145 | SRP1848-D02 | CDR-H2 | Chothia | TPQDGY |
| 146 | SRP1848-D03 | CDR-H2 | Chothia | FPNDGS |
| 147 | SRP1848-D04 | CDR-H2 | Chothia | YPRDGI |
| 148 | SRP1848-D05 | CDR-H2 | Chothia | SPIDGY |
| 149 | SRP1848-D07 | CDR-H2 | Chothia | SPNDGY |
| 150 | SRP1848-D09 | CDR-H2 | Chothia | SPNDGY |
| 151 | SRP1848-D10 | CDR-H2 | Chothia | SPNDGT |
| 152 | SRP1848-E01 | CDR-H2 | Chothia | TPFDGF |
| 153 | SRP1848-E02 | CDR-H2 | Chothia | YPNDGN |
| 154 | SRP1848-E03 | CDR-H2 | Chothia | TPRDGF |
| 155 | SRP1848-E05 | CDR-H2 | Chothia | TPNDGY |
| 156 | SRP1848-E06 | CDR-H2 | Chothia | TPNDGY |
| 157 | SRP1848-E07 | CDR-H2 | Chothia | FPYDGS |
| 158 | SRP1848-F01 | CDR-H2 | Chothia | FPNDGT |
| 159 | SRP1848-F02 | CDR-H2 | Chothia | FPNDGT |
| 160 | SRP1848-F04 | CDR-H2 | Chothia | YPIDGI |
| 161 | SRP1848-F05 | CDR-H2 | Chothia | YPNDGS |
| 162 | SRP1848-F06 | CDR-H2 | Chothia | SPIDGN |
| 163 | SRP1848-F07 | CDR-H2 | Chothia | YPNDGI |
| 164 | SRP1848-F08 | CDR-H2 | Chothia | YPVDGI |
| 165 | SRP1848-F09 | CDR-H2 | Chothia | SPLDGY |
| 166 | SRP1848-F10 | CDR-H2 | Chothia | FPNDGY |
| 167 | SRP1848-F11 | CDR-H2 | Chothia | TPIDGN |
| 168 | SRP1848-G01 | CDR-H2 | Chothia | APNDGS |
| 169 | SRP1848-G03 | CDR-H2 | Chothia | TPSDGF |
| 170 | SRP1848-G04 | CDR-H2 | Chothia | TPAGGA |
| 171 | SRP1848-G06 | CDR-H2 | Chothia | TPAGGA |
| 172 | SRP1848-G07 | CDR-H2 | Chothia | TPAGGA |
| 173 | SRP1848-G09 | CDR-H2 | Chothia | TPAGGA |
| 174 | SRP1848-G10 | CDR-H2 | Chothia | TPAGGA |
| 175 | SRP1848-G11 | CDR-H2 | Chothia | TPAGGA |
| 176 | SRP1848-H01 | CDR-H2 | Chothia | FPIDGI |
| 177 | SRP2060-E10 | CDR-H2 | Chothia | WWDDD |
| 178 | SRP2060-E05 | CDR-H2 | Chothia | WWDDD |
| 179 | SRP2060-B01 | CDR-H2 | Chothia | WWDDD |
| 180 | SRP2060-A06 | CDR-H2 | Chothia | WWDDD |
| 181 | SRP1848-A01 | CDR-H2 | Kabat | GILPESGGTSYADSVKG |
| 182 | SRP1848-A02 | CDR-H2 | Kabat | GIYPESGATYYADSVKG |
| 183 | SRP1848-A04 | CDR-H2 | Kabat | VIYPVDGTTDYADSVKG |
| 184 | SRP1848-A06 | CDR-H2 | Kabat | GITPIDGNTDYADSVKG |
| 185 | SRP1848-A07 | CDR-H2 | Kabat | EIFPVDGTTDYADSVKG |
| 186 | SRP1848-A08 | CDR-H2 | Kabat | SIYPNDGTTDYADSVKG |
| 187 | SRP1848-A09 | CDR-H2 | Kabat | SIFPNDGTTDYADSVKG |
| 188 | SRP1848-A10 | CDR-H2 | Kabat | DIFPIDDITDYADSVKG |
| 189 | SRP1848-B01 | CDR-H2 | Kabat | EIYPVDGITDYADSVKG |
| 190 | SRP1848-B03 | CDR-H2 | Kabat | GITPIDGMTDYADSVKG |
| 191 | SRP1848-B04 | CDR-H2 | Kabat | EIYPVDGITDYADSVKG |
| 192 | SRP1848-B05 | CDR-H2 | Kabat | GISPIDGYTDYADSMKG |
| 193 | SRP1848-B06 | CDR-H2 | Kabat | GITPIDGYTDYADSVKG |
| 194 | SRP1848-B07 | CDR-H2 | Kabat | GISPYDGFTDYADSVKG |
| 195 | SRP1848-B09 | CDR-H2 | Kabat | GITPVDGYTDYADRVKG |
| 196 | SRP1848-B10 | CDR-H2 | Kabat | EIYPRDGITDYADSVKG |
| 197 | SRP1848-B11 | CDR-H2 | Kabat | DISPIDGFTDYADSVKG |
| 198 | SRP1848-001 | CDR-H2 | Kabat | GVTPNDGYTDYADSVKG |
| 199 | SRP1848-0O3 | CDR-H2 | Kabat | EIYPIDGNTDYADSVKG |
| 200 | SRP1848-004 | CDR-H2 | Kabat | ElYPGPGNTDYADSVKG |
| 201 | SRP1848-005 | CDR-H2 | Kabat | DIFPIDGINDYADSVKG |
| 202 | SRP1848-007 | CDR-H2 | Kabat | DIFPIDGITDYADSVKG |
| 203 | SRP1848-C10 | CDR-H2 | Kabat | GISPIDGYTDYADSMKG |
| 204 | SRP1848-D02 | CDR-H2 | Kabat | GITPQDGYTDYADSVKG |
| 205 | SRP1848-D03 | CDR-H2 | Kabat | DIFPNDGSTDYADSVKG |
| 206 | SRP1848-D04 | CDR-H2 | Kabat | EIYPRDGITDYADSVKG |
| 207 | SRP1848-D05 | CDR-H2 | Kabat | GISPIDGYTDYADSVKG |
| 208 | SRP1848-D07 | CDR-H2 | Kabat | GISPNDGYTDYADSVKG |
| 209 | SRP1848-D09 | CDR-H2 | Kabat | GISPNDGYTDYADSVKG |
| 210 | SRP1848-D10 | CDR-H2 | Kabat | WISPNDGTTDYADSVKG |
| 211 | SRP1848-E01 | CDR-H2 | Kabat | GITPFDGFTDYADSVKG |
| 212 | SRP1848-E02 | CDR-H2 | Kabat | EIYPNDGNTDYADSVKG |
| 213 | SRP1848-E03 | CDR-H2 | Kabat | GITPRDGFTDYADSVKG |
| 214 | SRP1848-E05 | CDR-H2 | Kabat | GITPNDGYTDYADSVKG |
| 215 | SRP1848-E06 | CDR-H2 | Kabat | GITPNDGYTDYADSVEG |
| 216 | SRP1848-E07 | CDR-H2 | Kabat | EIFPYDGSTDYADNVKG |
| 217 | SRP1848-F01 | CDR-H2 | Kabat | SIFPNDGTTDYADSVKG |
| 218 | SRP1848-F02 | CDR-H2 | Kabat | SIFPNDGTTDYADSVKG |
| 219 | SRP1848-F04 | CDR-H2 | Kabat | EIYPIDGITDYADSVKG |
| 220 | SRP1848-F05 | CDR-H2 | Kabat | EIYPNDGSTDYADSVKG |
| 221 | SRP1848-F06 | CDR-H2 | Kabat | GISPIDGNTDYADSVKG |
| 222 | SRP1848-F07 | CDR-H2 | Kabat | EIYPNDGITDYADSVKG |
| 223 | SRP1848-F08 | CDR-H2 | Kabat | EIYPVDGITDYADSVKG |
| 224 | SRP1848-F09 | CDR-H2 | Kabat | GISPLDGYTDYADSVKG |
| 225 | SRP1848-F10 | CDR-H2 | Kabat | SIFPNDGYTDYADSVKG |
| 226 | SRP1848-F11 | CDR-H2 | Kabat | GITPIDGNTDYADSVKG |
| 227 | SRP1848-G01 | CDR-H2 | Kabat | WIAPNDGSTDYADSVKG |
| 228 | SRP1848-G03 | CDR-H2 | Kabat | GITPSDGFTDYADSVKG |
| 229 | SRP1848-G04 | CDR-H2 | Kabat | YITPAGGATFYADSVKG |
| 230 | SRP1848-G06 | CDR-H2 | Kabat | YITPAGGATYYADNVKG |
| 231 | SRP1848-G07 | CDR-H2 | Kabat | YITPAGGATWYADSVKG |
| 232 | SRP1848-G09 | CDR-H2 | Kabat | YITPAGGATFYADSVKG |
| 233 | SRP1848-G10 | CDR-H2 | Kabat | YITPAGGATYYADSVKG |
| 234 | SRP1848-G11 | CDR-H2 | Kabat | YITPAGGATWYADSVKG |
| 235 | SRP1848-H01 | CDR-H2 | Kabat | DIFPIDGITDYADSVKG |
| 236 | SRP2060-E10 | CDR-H2 | Kabat | HIWWDDDKYYHPALKG |
| 237 | SRP2060-E05 | CDR-H2 | Kabat | HIWWDDDKYYHPALKG |
| 238 | SRP2060-B01 | CDR-H2 | Kabat | HIWWDDDKYYHPALKG |
| 239 | SRP2060-A06 | CDR-H2 | Kabat | HIWWDDDKYYYPALKG |
| 240 | SRP1848-A01 | CDR-H3 | | HIYPWDWFSNYVLDY |
| 241 | SRP1848-A02 | CDR-H3 | | HLYVWDWVLDHVLDY |
| 242 | SRP1848-A04 | CDR-H3 | | GAWSWRSGYGYYIDY |
| 243 | SRP1848-A06 | CDR-H3 | | GAWSWRSGYGYYIDY |
| 244 | SRP1848-A07 | CDR-H3 | | GFWAWRSGYGYYLDY |
| 245 | SRP1848-A08 | CDR-H3 | | GSWFWRAGYGYYLDY |
| 246 | SRP1848-A09 | CDR-H3 | | GSWFWRSGYGYFLEY |
| 247 | SRP1848-A10 | CDR-H3 | | GSWSWPSGHSYYLDY |
| 248 | SRP1848-B01 | CDR-H3 | | GFWSWPSGYSYFLDY |
| 249 | SRP1848-B03 | CDR-H3 | | GSWSWPSGYSYYLDY |
| 250 | SRP1848-B04 | CDR-H3 | | GRYSWRAGYSYYLDY |
| 251 | SRP1848-B05 | CDR-H3 | | GSWFWQSGYGYYLDY |
| 252 | SRP1848-B06 | CDR-H3 | | GFWSWPSGYGYYQDY |
| 253 | SRP1848-B07 | CDR-H3 | | GSWSWPAGYGYYQDY |
| 254 | SRP1848-B09 | CDR-H3 | | GAWSWRSGYGYYMDY |
| 255 | SRP1848-B10 | CDR-H3 | | GGWHWRSGYSYYLDY |
| 256 | SRP1848-B11 | CDR-H3 | | GSWSWRAGYGYYLDY |
| 257 | SRP1848-C01 | CDR-H3 | | GSWFWRAGYGYYLDY |
| 258 | SRP1848-C03 | CDR-H3 | | GSWAWRSGYSYYLDY |
| 259 | SRP1848-C04 | CDR-H3 | | GSLSWRAGYGYYLDY |
| 260 | SRP1848-C05 | CDR-H3 | | GSWSWKAGYGYYLDY |
| 261 | SRP1848-C07 | CDR-H3 | | GSWSWPAGYGYYQDY |
| 262 | SRP1848-C10 | CDR-H3 | | GSWSWPAGYGYYLDY |
| 263 | SRP1848-D02 | CDR-H3 | | GAWSWRAGYGYYLDY |
| 264 | SRP1848-D03 | CDR-H3 | | GHWSWPSGYWYYLDY |
| 265 | SRP1848-D04 | CDR-H3 | | GYWFWRSGYGYYLDY |
| 266 | SRP1848-D05 | CDR-H3 | | GSWSWRAGYGYYLDY |
| 267 | SRP1848-D07 | CDR-H3 | | GFWAWRSGYGYYLDY |
| 268 | SRP1848-D09 | CDR-H3 | | GSWSWRHGYGYYLDY |
| 269 | SRP1848-D10 | CDR-H3 | | GAWSWRSGYGYYIDY |
| 270 | SRP1848-E01 | CDR-H3 | | GSWSWPAGYGYYQDY |
| 271 | SRP1848-E02 | CDR-H3 | | GSWSWRSGYGYYLDY |
| 272 | SRP1848-E03 | CDR-H3 | | GSWSWPAGHSYYLDY |
| 273 | SRP1848-E05 | CDR-H3 | | GFWAWRSGYGYYLDY |
| 274 | SRP1848-E06 | CDR-H3 | | GTWSWPSGHSYYLDY |
| 275 | SRP1848-E07 | CDR-H3 | | GAWSWRSGYGYYIDY |
| 276 | SRP1848-F01 | CDR-H3 | | GSWAWRAGYSYYLDY |
| 277 | SRP1848-F02 | CDR-H3 | | GSWSWQAGYGYYLDY |
| 278 | SRP1848-F04 | CDR-H3 | | GSWFWRSGYGYYLDY |
| 279 | SRP1848-F05 | CDR-H3 | | GSWAWRSGYSYFLDY |
| 280 | SRP1848-F06 | CDR-H3 | | GFWAWRSGYGYYLDY |
| 281 | SRP1848-F07 | CDR-H3 | | GSWDWRSGYSYYLDY |
| 282 | SRP1848-F08 | CDR-H3 | | GSWYWQSGYSYYLDY |
| 283 | SRP1848-F09 | CDR-H3 | | GAWSWRSGYGYYIDY |
| 284 | SRP1848-F10 | CDR-H3 | | GSWFWRSGYGYYLDY |
| 285 | SRP1848-F11 | CDR-H3 | | GSWYWRAGYGYYLDY |
| 286 | SRP1848-G01 | CDR-H3 | | GSWAWRSGYSYFLDY |
| 287 | SRP1848-G03 | CDR-H3 | | GSWSWPSGHGYFLDY |
| 288 | SRP1848-G04 | CDR-H3 | | YPYWFAGYMDY |
| 289 | SRP1848-G06 | CDR-H3 | | QPYWFAGYMDY |
| 290 | SRP1848-G07 | CDR-H3 | | YPFWFAGYMDY |
| 291 | SRP1848-G09 | CDR-H3 | | HEYWFSGYMDY |
| 292 | SRP1848-G10 | CDR-H3 | | YPYWFAGYIDY |
| 293 | SRP1848-G11 | CDR-H3 | | YPYWFSGYMDY |
| 294 | SRP1848-H01 | CDR-H3 | | GSWSWPSGMDYYLDY |
| 295 | SRP2060-E10 | CDR-H3 | | NHFPHYYGSSHWYFNV |
| 296 | SRP2060-E05 | CDR-H3 | | NHFPHYYGSSHWYFNV |
| 297 | SRP2060-B01 | CDR-H3 | | NHFPHYYGSSHWYFNV |
| 298 | SRP2060-A06 | CDR-H3 | | NHFPHYYGSSHWYFDV |
| 299 | trastuzumab | CDR-LI | | RASQDVNTAVA |
| 300 | H6D1-LC4 | CDR-L1 | | KASQDINSYLS |
| 301 | H6D1-LC5 | CDR-L1 | | KASQDINSYLS |
| 302 | trastuzumab | CDR-L2 | | SASFLYS |
| 303 | H6D1-LC4 | CDR-L3 | | RANRLVD |
| 304 | H6D1-LC5 | CDR-L2 | | RANRLVD |
| 305 | trastuzumab | CDR-L3 | | QQHYTTPPT |
| 306 | H6D1-LC4 | CDR-L3 | | LQYDEFPYT |
| 307 | H6D1-LC5 | CDR-L3 | | LQYDEFPYT |
| 308 | SRP1848-A01 | VH | | EVQLVESGGGLVQPGGSLRLSCAASGFN |
| | | | | ITRYSIHWVRQAPGKGLEWVAGILPESG |
| | | | | GTSYADSVKGRFTISADTSKNTAYLQMN |
| | | | | SLRAEDTAVYYCARHIYPWDWFSNYVLD |
| | | | | YWGQGTLVTVSS |
| 309 | SRP1848-A02 | VH | | EVQLVESGGGLVQPGGSLRLSCAASGFN |
| | | | | ISGFRIHWVRQAPGKGLEWVAGIYPESG |
| | | | | ATYYADSVKGRFTISADTSKNTAYLQMN |
| | | | | SLRAEDTAVYYCARHLYVWDWVLDHVLD |
| | | | | YWGQGTLVTVSS |
| 310 | SRP1848-A04 | VH | | EVQLVESGGGLVQPGGSLRLSCAASGFN |
| | | | | ISSIDQHWVRQAPGKGLEWVGVIYPVDG |
| | | | | TTDYADSVKGRFTISADTSKNTAYLQMN |
| | | | | SLRAEDTAVYYCARGAWSWRSGYGYYID |
| | | | | YWGQGTLVTVSS |
| 311 | SRP1848-A06 | VH | | EVQLVESGGGLVQPGGSLRLSCAASGFN |
| | | | | IGNSYIHWVRQAPGKGLEWVGGITPIDG |
| | | | | NTDYADSVKGRFTISADTSKNTAYLQMN |
| | | | | SLRAEDTAVYYCARGAWSWRSGYGYYID |
| | | | | YWGQGTLVTVSS |
| 312 | SRP1848-A07 | VH | | EVQLVESGGGLVQPGGSLRLSCAASGFN |
| | | | | IGYHSIHWVRQAPGKGLEWVGEIFPVDG |
| | | | | TTDYADSVKGRFTISADTSKNTAYLHMN |
| | | | | SLRAEDTAVYYCARGFWAWRSGYGYYLD |
| | | | | YWGQGTLVTVSS |
| 313 | SRP1848-A08 | VH | | EVQLVESGGGLVQPGGSLRLSCAASGSN |
| | | | | IRKHSIHWVRQAPGKGLEWVGSIYPNDG |
| | | | | TTDYADSVKGRFTISADTSKNTAYLQMN |
| | | | | SLRAEDTAVYYCARGSWFWRAGYGYYLD |
| | | | | YWGQGTLVTVSS |
| 314 | SRP1848-A09 | VH | | EVQLVESGGGLVQPGGSLRLSCAASGFN |
| | | | | IRKQSIHWVRQAPGKGLEWVGSIFPNDG |
| | | | | TTDYADSVKGRFTISADTSKNTAYLQVN |
| | | | | SLRAEDTAVYYCARGSWFWRSGYGYFLE |
| | | | | YWGQGTLVTVSS |
| 315 | SRP1848-A10 | VH | | EVQLVESGGGLVQPGGSLRLSCAASGFN |
| | | | | IRKYSIHWARQAPGKGLEWVGDIFPIDD |
| | | | | ITDYADSVKGRFTISADTSKNTAYLQMN |
| | | | | SLRAEDTAVYYCARGSWSWPSGHSYYLD |
| | | | | YWGQGTLVTVSS |
| 316 | SRP1848-B01 | VH | | EVQLVESGGGLVQPGGSLRLSCAASGFN |
| | | | | IRNYSIHWVRQAPGKGLEWVGEIYPVDG |
| | | | | ITDYADSVKGRFTISADTSKNTAYLQMN |
| | | | | SLRAEDTAVYYCARGFWSWPSGYSYFLD |
| | | | | YWGQGTLVTVSS |
| 317 | SRP1848-B03 | VH | | EVQLVESGGGLVQPGGSLRLSCAASGFN |
| | | | | ISMKYIHWVRQAPGKGLEWVGGITPIDG |
| | | | | MTDYADSVKGRFTISADTSKNTAYLQMN |
| | | | | SLRAEDTAVYYCARGSWSWPSGYSYYLD |
| | | | | YWGQGTLVTVSS |
| 318 | SRP1848-B04 | VH | | EVQLVESGGGLVQPGGSLRLSCAASSFN |
| | | | | ISNHSIHWVRQAPGKGLEWVGEIYPVDG |
| | | | | ITDYADSVKGRFTISADTSKNTAYLQMN |
| | | | | SLRAEDTAVYYCARGRYSWRAGYSYYLD |
| | | | | YWGQGTLVTVSS |
| 319 | SRP1848-B05 | VH | | EVQLVESGGGLVQPGGSLRLSCAASGFN |
| | | | | ISNYYIHWVRQAPGKGLEWVGGISPIDG |
| | | | | YTDYADSMKGRFISADTSKNTAYLQMS |
| | | | | SLRAEDTAVYYCARGSWFWQSGYGYYLD |
| | | | | YWGQGTLVTVSS |
| 320 | SRP1848-B06 | VH | | EVQLVESGGGLVQPGGSLRLSCAASGFN |
| | | | | ISNYYIHWVRQAPGKGLEWVGGITPIDG |
| | | | | YTDYADSVKGRFTISADTSKNTAYLQMN |
| | | | | SLRAEDTAVYYCARGFWSWPSGYGYYQD |
| | | | | YWGQGTLVTVSS |
| 321 | SRP1848-B07 | VH | | EVQLVESGGGLVQPGGSLRLSCAASGFN |
| | | | | ISRFYIHWVRQAPGKGLEWVGGISPYDG |
| | | | | FTDYADSVKGRFTISADTSKNTAYLQMN |
| | | | | SLRAEDTAVYYCARGSWSWPAGYGYYQD |
| | | | | YWGQGTLVTVSS |
| 322 | SRP1848-B09 | VH | | EVQLVESGGGLVQPGGSLRLSCAAGGFN |
| | | | | ITNYYIHWVRQAPGKGLEWVGGITPVDG |
| | | | | YTDYADRVKGRFTISADTSKNTAYLQMN |
| | | | | SLRAEDTAVYYCARGAWSWRSGYGYYMD |
| | | | | YWGQGTLVTVSS |
| 323 | SRP1848-B10 | VH | | EVQLVESGGGLVQPGGSLRLSCAASGFN |
| | | | | TTTKSIHWVRQAPGKGLEWVGEIYPRDG |
| | | | | ITDYADSVKGRFTISADTSKNTAYLQMN |
| | | | | SLRAEDTAVYYCARGGWHWRSGYSYYLD |
| | | | | YWGQGTLVTVSS |
| 324 | SRP1848-B11 | VH | | EVQLVESGGGLVQPGGSLRLSCAASGFN |
| | | | | IGNNSIHWVRQAPGKGLEWVGDISPIDG |
| | | | | FTDYADSVKGRFTISADTSKNTAYLQMN |
| | | | | SLRAEDTAVYYCARGSWSWRAGYGYYLD |
| | | | | YWGQGTLVTVSS |
| 325 | SRP1848-C01 | VH | | EVQLVESGGGLVQPGGSLRLSCAASGFN |
| | | | | IGNSYIHWVRQAPGKGLEWVGGVTPNDG |
| | | | | YTDYADSVKGRFTISADTSKNTTYLQMN |
| | | | | SLRAEDTAVYYCARGSWFWRAGYGYYLD |
| | | | | YWGQGALVTVSS |
| 326 | SRP1848-C03 | VH | | EVQLVESGGGLVQPGGSLRLSCAASGFN |
| | | | | IGVYSIHWVRQAPGKGLEWVGEIYPIDG |
| | | | | NTDYADSVKGRFTISADTSKNTAYLQMN |
| | | | | SLRAEDTAVYYCARGSWAWRSGYSYYLD |
| | | | | YWGQGTLVTVSS |
| 327 | SRP1848-C04 | VH | | EVQLVESGGGLVQPGGSLRLSCAASGFN |
| | | | | IRHYSIHWVRQAPGKGLEWVGEIYPGPG |
| | | | | NTDYADSVKGRFTISADTSKNTAYLQMN |
| | | | | SLRAEDTAVYYCARGSLSWRAGYGYYLD |
| | | | | YWGQGTLVTVSS |
| 328 | SRP1848-C05 | VH | | EVQLVESGGGLVQPGGSLRLSCAASGFN |
| | | | | IRKYSIHWVRQAPGKGLEWVGDIFPIDG |
| | | | | INDYADSVKGRFTISADTSKNTAYLQMN |
| | | | | SLRAEDTAVYYCARGSWSWKAGYGYYLD |
| | | | | YWGQGTLVTVSS |
| 329 | SRP1848-C07 | VH | | EVQLVESGGGLVQPGGSLRLSCAASGFN |
| | | | | IRKYSIHWVRQAPGKGLEWVGDIFPIDG |
| | | | | ITDYADSMKGRFTISADTSKNTAYLQMN |
| | | | | SLRAEDTAVYYCARGSWSWPAGYGYYQD |
| | | | | YWGQGTLVTVSS |
| 330 | SRP1848-C10 | VH | | EVQLVESGGGLVQPGGSLRLSCAASGFN |
| | | | | IRTYYIHWVRQAPGKGLEWVGGISPIDG |
| | | | | YTDYADSVKGRFTISADTSKNTAYLQMN |
| | | | | SLRAEDTAVYYCARGSWSWPAGYGYYLD |
| | | | | YWGQGTLVTVSS |
| 331 | SRP1848-D02 | VH | | EVQLVESGGGLVQPGGSLRLSCAASGFN |
| | | | | ISHNYIHWVRQAPGKGLEWVGGITPQDG |
| | | | | YTDYADSVKGRFTISADTSKNTAYLQMN |
| | | | | RLRAEDTAVYYCARGAWSWRAGYGYYLD |
| | | | | YWGQGTLVTVSS |
| 332 | SRP1848-D03 | VH | | EVQLVESGGGVVQPGGSLRLSCAASGFN |
| | | | | IRYFSIHWVRQAPGKGLEWVGDIFPNDG |
| | | | | STDYADSVKGRFTISADTSKNTAYLQMN |
| | | | | SLRAEETAVYYCARGHWSWPSGYWYYLD |
| | | | | YWGQGTLVTVSS |
| 333 | SRP1848-D04 | VH | | EVQLVESGGGLVQPGGSLRLSCAASGFN |
| | | | | ISHYSIHWVRQAPGKGLEWVGEIYPRDG |
| | | | | ITDYADSVKGRFTISADTSKNTAYLQMN |
| | | | | SLSAEDTAVYYCARGYWFWRSGYGYYLD |
| | | | | YWGQGTLVTVSS |
| 334 | SRP1848-D05 | VH | | EVQLVESGGGLVQPGGSLRLSCAASGFN |
| | | | | ISISYIHWVRQAPGKGLEWVGGISPIDG |
| | | | | YTDYADSVKGRFTISADTSKNTAYLQMN |
| | | | | SLRAEDTAVYYCARGSWSWRAGYGYYLD |
| | | | | YWGQGTLVTVSS |
| 335 | SRP1848-D07 | VH | | EVQLVESGGGLVQPGGSLRLSCAASGFN |
| | | | | ISKYYIHWVRQAPGKGLEWVGGISPNDG |
| | | | | YTDYADSVKGRFAISADTSKNTAYLQMN |
| | | | | SLRAEDTAVYYCARGFWAWRSGYGYYLD |
| | | | | YWGQGTLVTVSS |
| 336 | SRP1848-D09 | VH | | EVQLVESGGGLVQPGGSLRLSCAASGFN |
| | | | | ISNYYIHWVRQAPGKGLEWVGGISPNDG |
| | | | | YTDYADSVKGRFTISADTSKNTAYLQMN |
| | | | | SLRAEDTAVYYCARGSWSWRHGYGYYLD |
| | | | | YWGQGTLVTVSS |
| 337 | SRP1848-D10 | VH | | EVQLVESGGGLVQPGGSLRLSCAASGFN |
| | | | | ISRNSIHWVRQAPGKGLEWVGWISPNDG |
| | | | | TTDYADSVKGRFTISADGSKNTAYLQMN |
| | | | | SLRAEDTAVYYCARGAWSWRSGYGYYID |
| | | | | YWGQGTLVTVSS |
| 338 | SRP1848-E01 | VH | | EVQLVESGGGLVQPGGSLRLSCAASGFN |
| | | | | ITNKYIHWVRQAPGKGLEWVGGITPFDG |
| | | | | FTDYADSVKGRFTISADTSKNTAYLQMN |
| | | | | SLRAEDTAVYYCARGSWSWPAGYGYYQD |
| | | | | YWGQGTLVTVSS |
| 339 | SRP1848-E02 | VH | | EVQLVESGGGLVQPGGSLRLSCAASGFN |
| | | | | IGKYSIHWVRQAPGKGLEWVGEIYPNDG |
| | | | | NTDYADSVKGRFTISADTSKNTAYLQMN |
| | | | | SLRAEDTAVYYCARGSWSWRSGYGYYLD |
| | | | | YWGQGTLVTVSS |
| 340 | SRP1848-E03 | VH | | EVQLVESGGGLAQPGGSLRLSCAASGFN |
| | | | | IGNYYIHWVRQAPGKGLEWVGGITPRDG |
| | | | | FTDYADSVKGRFTISADTSKNTAYLQVN |
| | | | | SLRAEDTAVYYCARGSWSWPAGHSYYLD |
| | | | | YWGQGTLVTVSS |
| 341 | SRP1848-E05 | VH | | EVQLVESGGGLVQPGGSLRVSCAASGFN |
| | | | | IGVYYIHWVRQAPGKGLEWVGGITPNDG |
| | | | | YTDYADSVKGRFTISADTSKNTAYLQMN |
| | | | | SLRAEDTAVYYCARGFWAWRSGYGYYLD |
| | | | | YWGQGTLVTVSS |
| 342 | SRP1848-E06 | VH | | EVQLVESGGGLVQPSGSLRLSCAASGFN |
| | | | | INRYYIHWVRQAPGKGLEWVGGITPNDG |
| | | | | YTDYADSVEGRFTTSADTSKNTAYLQMN |
| | | | | SLRAEDTAVYYCARGTWSWPSGHSYYLD |
| | | | | YWGQGTLVTVSS |
| 343 | SRP1848-E07 | VH | | EVQLVESGGGLVQPGGSLRLSCAASGFN |
| | | | | IRKSSIHWVRQAPGKGLEWVGEIFPYDG |
| | | | | STDYADNVKGRFTISADTSKNTAYLQMN |
| | | | | SLRAEDTAVYYCARGAWSWRSGYGYYID |
| | | | | YWGQGTLVTVSS |
| 344 | SRP1848-F01 | VH | | EVQLVESGGGLVQPGGSLRLSCAASGFN |
| | | | | IRTYSIHWVRQAPGKGLEWVGSIFPNDG |
| | | | | TTDYADSVKGRFTISADTSKNTAYLQMN |
| | | | | SLRAEDTAVYYCARGSWAWRAGYSYYLD |
| | | | | YWGQGTLVTVSS |
| 345 | SRP1848-F02 | VH | | EVQLVESGGGLVQPGGSLRLSCAASGFN |
| | | | | IRTYSIHWVRQAPGKGLEWVGSIFPNDG |
| | | | | TTDYADSVKGRLTISADTSKNTAYLQMN |
| | | | | SLRAEDTAVYYCARGSWSWQAGYGYYLD |
| | | | | YWGQGTLVTVSS |
| 346 | SRP1848-F04 | VH | | EVQLVESGGGLVQPGGSLRLSCAASGFN |
| | | | | ISNYSIHWVRQAPGKGLEWVGEIYPIDG |
| | | | | ITDYADSVKGRFTISADTSKNTAYLQMN |
| | | | | SLRAEDTAVYYCARGSWFWRSGYGYYLD |
| | | | | YWGQGTLVTVSS |
| 347 | SRP1848-F05 | VH | | EVQLVESGGGLVQPGGSLRLSCAASGFN |
| | | | | ISKSSIHWVRQAPGKGLEWVGEIYPNDG |
| | | | | STDYADSVKGRFTISADTSKNTAYLQMN |
| | | | | SLRAEDTAVYYCARGSWAWRSGYSYFLD |
| | | | | YWGQGTLVTVSS |
| 348 | SRP1848-F06 | VH | | EVQLVESGGGLVQPGGSLRLSCAASGFN |
| | | | | ISLSYIHWVRQAPGKGLEWVGGISPIDG |
| | | | | NTDYADSVKGRFTISADTSKNTAYLQMN |
| | | | | SLRAEDTAVYYCARGFWAWRSGYGYYLD |
| | | | | YWGQGTLVTVSS |
| 349 | SRP1848-F07 | VH | | EVQLVESGGGLVQPGGSLRLSCAASGFN |
| | | | | ISNHSIHWVRQAPGKGLEWVGEIYPNDG |
| | | | | ITDYADSVKGRFTISADTSKNTAYLQMN |
| | | | | SLSAEDTAVYYCARGSWDWRSGYSYYLD |
| | | | | YWGQGTLVTVSS |
| 350 | SRP1848-F08 | VH | | EVQLVESGGGLVQPGGSLRLSCAAGGFN |
| | | | | ISNHSIHWVRQAPGKGVEWVGEIYPVDG |
| | | | | ITDYADSVKGRFTISADTSKNTAYLRMN |
| | | | | SLRAEDTAVYYCARGSWYWQSGYSYYLD |
| | | | | YWGQGTLVTVSS |
| 351 | SRP1848-F09 | VH | | EVQLVESGGGLVQPGGSLRLSCAASGFN |
| | | | | ISNHYIHWVRQAPGKGLEWVGGISPLDG |
| | | | | YTDYADSVKGRFTISADTSKNTAYLQMN |
| | | | | SLRAEDTAVYYCARGAWSWRSGYGYYID |
| | | | | YWGQGTLVTVSS |
| 352 | SRP1848-F10 | VH | | EVQLVESGGGLVQPGGSLRLSCAASGFN |
| | | | | ISNNSIHWVRQAPGKGLEWVGSIFPNDG |
| | | | | YTDYADSVKGRFTISADTSKNTAYLQMN |
| | | | | SLRAEDTAVYYCARGSWFWRSGYGYYLD |
| | | | | YWGQGTLVTVSS |
| 353 | SRP1848-F11 | VH | | ISNNYIHWVRQAPGKGLEWVGGITPIDG |
| | | | | NTDYADSVKGRFTISADTSMNTAYLQMN |
| | | | | SLRAEDTAVYYCARGSWYWRAGYGYYLD |
| | | | | YWGQGALVTVSS |
| 354 | SRP1848-G01 | VH | | EVQLVESGGGLVQPGGSLRLSCAASGFN |
| | | | | ISRHSIHWVRQAPGKGLEWVGWIAPNDG |
| | | | | STDYADSVKGRFTISADTSKNTAYLQMN |
| | | | | SLRAEDTAVYYCARGSWAWRSGYSYFLD |
| | | | | YWGQGTLVTVSS |
| 355 | SRP1848-G03 | VH | | ISTYYIHWVRQAPGKGLEWVGGITPSDG |
| | | | | FTDYADSVKGRSTISADTSKNTAYLQMN |
| | | | | SLRAEDTAVYYCARGSWSWPSGHGYFLD |
| | | | | YWGQGTLVTVSS |
| 356 | SRP1848-G04 | VH | | EVQLVESGGGLVQPGGSLRLSCAASGFN |
| | | | | IHSTDIHWVRQAPGKGLEWVAYITPAGG |
| | | | | ATFYADSVKGRFTISADTSKNTAYLQMN |
| | | | | SLRAEDTAVYYCARYPYWFAGYMDYWGQ |
| | | | | GTLVTVSS |
| 357 | SRP1848-G06 | VH | | EVQLVESGGGLVQPGGSLRLSCAASGFN |
| | | | | IRSTDIHWVRQAPGKGLEWVAYITPAGG |
| | | | | ATYYADNVKGRFTISADTSKNTAYLQMN |
| | | | | SLRAEDTAVYYCARQPYWFAGYMDYWGQ |
| | | | | GTLVTVSS |
| 358 | SRP1848-G07 | VH | | EVQLVESGGGLVQPGGSLRLSCAASGFN |
| | | | | IHSTDIHWVRQAPGKGLEWVAYITPAGG |
| | | | | ATWYADSVKGRFTISADTSKNTAYLQMN |
| | | | | SLRAEDTAVYYCARYPFWFAGYMDYWGQ |
| | | | | GTLVTVSS |
| 359 | SRP1848-G09 | VH | | EVQLVESGGGLVQPGGSLRLSCAASGFN |
| | | | | IRGTDIHWVRQAPGKGLEWVAYITPAGG |
| | | | | ATFYADSVKGRFTISADTSKNTAYLQMN |
| | | | | SLRAEDTAVYYCARHEYWFSGYMDYWGQ |
| | | | | GTLVTVSS |
| 360 | SRP1848-G10 | VH | | EVQLVESGGGLVQPGSSLRLSCAASGFN |
| | | | | IRSTDIHWVRQAPGKGLEWVAYITPAGG |
| | | | | ATYYADSVKGRFTISADTSKNTAYLQMN |
| | | | | SLRAEDTAVYYCARYPYWFAGYIDYWGQ |
| | | | | GTLVTVSS |
| 361 | SRP1848-G11 | VH | | EVQLVESGGGLVQPGGSLRLSCAASGFN |
| | | | | ISSTDIHWVRQAPGKGLEWVAYITPAGG |
| | | | | ATWYADSVKGRFTISADTSKNTAYLQMN |
| | | | | SLRAEDTAVYYCARYPYWFSGYMDYWGQ |
| | | | | GTLVTVSS |
| 362 | SRP1848-H01 | VH | | EVQLVESGGGLVQPGGSLRLSCAASGFN |
| | | | | IRTQSIHWVRQAPGKGLEWIGDIFPIDG |
| | | | | ITDYADSVKGRFTISADTSKNTAYLQMN |
| | | | | SLRAEDTAVYYCARGSWSWPSGMDYYLD |
| | | | | YWGQGTLVTVSS |
| 363 | SRP2060-E10 | VH | | EVQLLESGGGLVQPGGSLRLSCAFSGFS |
| | | | | LSTFGMGVGWVRQAPGKGLEWVSHIWWD |
| | | | | DDKYYHPALKGRFTISKDNSKNTVYLQM |
| | | | | NSLRAEDTAVYYCGRNHFPHYYGSSHWY |
| | | | | FNVWGQGTTVTVSS |
| 364 | SRP2060-E05 | VH | | EVQLLESGGGLVQPGGSLRLSCAFSGFS |
| | | | | LSTFGMGVGWVRQAPGKGLEWVSHIWWD |
| | | | | DDKYYHPALKGRFTVSKDNSKNTVYLQM |
| | | | | NSLRAEDTAVYYCGRNHFPHYYGSSHWY |
| | | | | FNVWGQGTTVTVSS |
| 365 | SRP2060-B01 | VH | | EVQLLESGGGLVQPGGSLRLSCALSGFS |
| | | | | LSTFGMGVGWVRQATGKGLEWVSHIWWD |
| | | | | DDKYYHPALKGRFTISKDNSKNTVHLQM |
| | | | | NSLRAEDTAVYYCGRNHFPHYYGSSHWY |
| | | | | FNVWGQGTTVTVSS |
| 366 | SRP2060-A06 | VH | | EVQLLESGGGLVQPGGSLRLSCAFSGFS |
| | | | | LSTFGMGVGWVRQAPGKGLEWVGHIWWD |
| | | | | DDKYYYPALKGRFTISKDNSKNTVYLQM |
| | | | | NSLRAEDTAVYYCGRNHFPHYYGSSHWY |
| | | | | FDVWGQGTTVTVSS |
| 367 | trastuzumab | VL | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIK |
| 368 | H6D1-LC4 | VL | | EIVMTQSPATLSLSPGERATLSCKASQD |
| | | | | INSYLSWYQQKPGQAPRLLIYRANRLVD |
| | | | | GIPARFSGSGSGTDYTLTISSLEPEDFA |
| | | | | VYYCLQYDEFPYTFGGGTKVEIK |
| 369 | H6D1-LC5 | VL | | DIQMTQSPSTLSASVGDRVTITCKASQD |
| | | | | INSYLSWYQQKPGKAPKLLIYRANRLVD |
| | | | | GVPSRFSGSGSGTEFTLTISSLQPDDFA |
| | | | | TYYCLQYDEFPYTFGGGTKVEIK |
| 370 | Human IgG1 HC | | | ASTKGPSVFPLAPSSKSTSGGTAALGCL |
| | Constant | | | VKDYFPEPVTVSWNSGALTSGVHTFPAV |
| | | | | LQSSGLYSLSSVVTVPSSSLGTQTYICN |
| | | | | VNHKPSNTKVDKKVEPKSCDKTHTCPPC |
| | | | | PAPELLGGPSVFLEPPKPKDTLMISRTP |
| | | | | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSREEMTKNQVSLTCLV |
| | | | | KGFYPSDIAVEWESNGQPENNYKTTPPV |
| | | | | LDSDGSFFLYSKLTVDKSRWQQGNVFSC |
| | | | | SVMHEALHNHYTQKSLSLSPGK |
| 371 | Human IgG LC | | | RTVAAPSVFIEPPSDEQLKSGTASVVCL |
| | Constant Ckappa | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 372 | Mouse IgG1 HC | | | AKTTPPSVYPLAPGSAAQTNSMVTLGCL |
| | Constant | | | VKGYFPEPVTVTWNSGSLSSGVHTFPAV |
| | | | | LQSDLYTLSSSVTVPSSTWPSETVTCNV |
| | | | | AHPASTKVDKKIVPRDCGCKPCICTVP |
| | | | | EVSSVFIFPPKPKDVLTITLTPKVTCVV |
| | | | | VDISKDDPEVQFSWFVDDVEVHTAQTQP |
| | | | | REEQFNSTFRSVSELPIMHQDWLNGKEF |
| | | | | KCRVNSAAFPAPIEKTISKTKGRPKAPQ |
| | | | | VYTIPPPKEQMAKDKVSLTCMITDFFPE |
| | | | | DITVEWQWNGQPAENYKNTQPIMDTDGS |
| | | | | YFVYSKLNVQKSNWEAGNTFTCSVLHEG |
| | | | | LHNHHTEKSLSHSPG |
| 373 | Mouse IgG LC | | | RADAAPTVSIEPPSSEQLTSGGASVVCF |
| | Constant Ckappa | | | LNNFYPKD NVKWKIDGSERQNGVLNSW |
| | | | | TDQDSKDSTYSMSSTLTLTKDEYERHNS |
| | | | | YTCEATHKTSTSPIVKSFNRNEC |
| 374 | Kappa LC | | | HMTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 375 | Lambda LD | | | GQPKAAPSVTLFPPSSEELQANKATLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 376 | FlagHis Tag | | | GSGDYKDDDDKGSGHHHHHH |
| 377 | Linker | | | GGGGSGGGGSGGGGS |
| 378 | Linker | | | AAGSDQEPKSS |
| 379 | 1848-B10-VH- | scFv | | MEVQLVESGGGLVQPGGSLRLSCAASGF |
| | (G4S)3-VL | | | NTTTKSIHWVRQAPGKGLEWVGEIYPRD |
| | | | | GITDYADSVKGRFTISADTSKNTAYLQM |
| | | | | NSLRAEDTAVYYCARGGWHWRSGYSYYL |
| | | | | DYWGQGTLVTVSSGGGGSGGGGSGGGGS |
| | | | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIK |
| 380 | 1848-B10-VL- | scFv | | MDIQMTQSPSSLSASVGDRVTITCRASQ |
| | (G4S)3-VH | | | DVNTAVAWYQQKPGKAPKLLIYSASFLY |
| | | | | SGVPSRFSGSRSGTDFTLTISSLQPEDF |
| | | | | ATYYCQQHYTTPPTFGQGTKVEIKGGGG |
| | | | | SGGGGSGGGGSEVQLVESGGGLVQPGGS |
| | | | | LRLSCAASGFNTTTKSIHWVRQAPGKGL |
| | | | | EWVGEIYPRDGITDYADSVKGRFTISAD |
| | | | | TSKNTAYLQMNSLRAEDTAVYYCARGGW |
| | | | | HWRSGYSYYLDYWGQGTLVTVSS |
| 381 | 1848-B10-VH- | scFv-Fc | | MEVQLVESGGGLVQPGGSLRLSCAASGF |
| | (G4S)3-VL | | | NTTTKSIHWVRQAPGKGLEWVGEIYPRD |
| | | | | GITDYADSVKGRFTISADTSKNTAYLQM |
| | | | | NSLRAEDTAVYYCARGGWHWRSGYSYYL |
| | | | | DYWGQGTLVTVSSGGGGSGGGGSGGGGS |
| | | | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIKAAGSD |
| | | | | QEPKSSDKTHTCPPCPAPELLGGPSVFL |
| | | | | FPPKPKDTLMISRTPEVTCVVVDVSHED |
| | | | | PEVKFNWYVDGVEVHNAKTKPREEQYNS |
| | | | | TYRVVSVLTVLHQDWLNGKEYKCKVSNK |
| | | | | ALPAPIEKTISKAKGQPREPQVYTLPPS |
| | | | | REEMTKNQVSLTCLVKGFYPSDIAVEWE |
| | | | | SNGQPENNYKTTPPVLDSDGSFFLYSKL |
| | | | | TVDKSRWQQGNVFSCSVMHEALHNHYTQ |
| | | | | KSLSLSPGK |
| 382 | 1848-B10-VL- | scFv-Fc | | MDIQMTQSPSSLSASVGDRVTITCRASQ |
| | (G4S)3-VH | | | DVNTAVAWYQQKPGKAPKLLIYSASFLY |
| | | | | SGVPSRFSGSRSGTDFTLTISSLQPEDF |
| | | | | ATYYCQQHYTTPPTFGQGTKVEIKGGGG |
| | | | | SGGGGSGGGGSEVQLVESGGGLVQPGGS |
| | | | | LRLSCAASGFNTTTKSIHWVRQAPGKGL |
| | | | | EWVGEIYPRDGITDYADSVKGRFTISAD |
| | | | | TSKNTAYLQMNSLRAEDTAVYYCARGGW |
| | | | | HWRSGYSYYLDYWGQGTLVTVSSAAGSD |
| | | | | QEPKSSDKTHTCPPCPAPELLGGPSVFL |
| | | | | FPPKPKDTLMISRTPEVTCVVVDVSHED |
| | | | | PEVKFNWYVDGVEVHNAKTKPREEQYNS |
| | | | | TYRVVSVLTVLHQDWLNGKEYKCKVSNK |
| | | | | ALPAPIEKTISKAKGQPREPQVYTLPPS |
| | | | | REEMTKNQVSLTCLVKGFYPSDIAVEWE |
| | | | | SNGQPENNYKTTPPVLDSDGSFFLYSKL |
| | | | | TVDKSRWQQGNVFSCSVMHEALHNHYTQ |
| | | | | KSLSLSPGK |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated antibody that specifically binds to folate receptor alpha (FOLR1), wherein the antibody comprises a CDR-H3 sequence selected from:
a sequence defined by the consensus sequence G-α₂-α₃-α₄-W-α₆-α₇-G-α₉-α₁₀-Y-α₁₂-α₁₃-α₁₄-Y, where α₂ is G, S, A, F, H, R, T, or Y; α₃ is W, L, or Y; α₄ is S, A, F, Y, H, or D; α₆ is R, P, Q, or K; α₇ is S, A, or H; α₉ is Y, H, or M; α₁₀ is G, S, D, or W; α₁₂ is Y or F; α₁₃ is L, I, Q, or M; and α₁₄ is D or E;
a sequence defined by the consensus sequence G-α₂-α₃-α₄-W-α₆-α₇-G-α₉-α₁₀-Y-α₁₂-α₁₃-α₁₄-Y, where α₂ is G or S; α₃ is W; α₄ is S or H; α₆ is R or P; α₇ is S; α₉ is Y or M; α₁₀ is G, S, or D; α₁₂ is Y; α₁₃ is L; and α₁₄ is D; and
a sequence selected from SEQ ID NOs: 240-298, or a variant thereof having three, two, or one amino acid substitution(s).

2. The antibody of claim 1, wherein the CDR-H3 sequence is a sequence selected from SEQ ID NOs: 240-298.

3. The antibody of claim 1, wherein the antibody comprises a Chothia CDR-H2 sequence selected from:
a sequence defined by the consensus sequence ε₁-ε₂-ε₃-ε₄-ε₅-ε₆, where ε₁ is Y, T, F, S, or A; ε₂ is P; ε₃ is N, I, V, R, Y, F, G, L, Q, or S; ε₄ is D or P; ε₅ is G or D; and ε₆ is Y, I, T, N, F, S, or M;
a sequence defined by the consensus sequence ε₁-ε₂-ε₃-ε₄-ε₅-ε₆, where ε₁ is Y or F; ε₂ is P; ε₃ is N, I, or R; ε₄ is D; ε₅ is G; and ε₆ is Y or I; and
a sequence selected from SEQ ID NOs: 122-180, or a variant thereof having two or one amino acid substitutions(s).

4. The antibody of claim 3, wherein the Chothia CDR-H2 sequence is a sequence selected from SEQ ID NOs: 122-180.

5. The antibody of claim 1, wherein the antibody comprises a Chothia CDR-H1 sequence selected from:
a sequence defined by the consensus sequence γ₁-γ₂-γ₃-γ₄-γ₅-γ₆-γ₇, where γ₁ is G or S; γ₂ is F or S; γ₃ is N; γ₄ is I or T; γ₅ is S, R, G, T, N, or D; γ₆ is N, K, T, R, H, Y, L, M, Q, or V; and γ₇ is Y, H, S, N, K, F, or Q;
a sequence defined by the consensus sequence γ₁-γ₂-γ₃-γ₄-γ₅-γ₆-γ₇, where γ₁ is G; γ₂ is F; γ₃ is N; γ₄ is I or T; γ₅ is S, R, or T; γ₆ is N or T; and γ₇ is Y, K, or Q; and
a sequence selected from SEQ ID NOs: 4-62, or a variant thereof having two or one amino acid substitutions(s).

6. The antibody of claim 5, wherein the Chothia CDR-H1 sequence is a sequence selected from SEQ ID NOs: 4-62.

7. The antibody of claim 1, wherein the antibody comprises a Kabat CDR-H2 sequence selected from:
a sequence defined by the consensus sequence θ₁-θ₂-θ₃-θ₄-θ₅-θ₆-θ₇-θ₈-θ₉-D-Y-A-D-θ₁₄-θ₁₅-θ₁₆-G, where θ₁ is G, E, D, W, S, or V; θ₂ is I or V; θ₃ is Y, T, F, S, or A; θ₄ is P; θ₅ is N, I, V, R, Y, F, G, L, Q, or S; θ₆ is D or P; θ₇ is G or D; θ₈ is Y, I, T, N, F, S, or M; θ₉ is T or N; θ₁₄ is S, R, or N; θ₁₅ is V or M; and θ₁₆ is K or E;
a sequence defined by the consensus sequence θ₁-θ₂-θ₃-θ₄-θ₅-θ₆-θ₇-θ₈-θ₉-D-Y-A-D-θ₁₄-θ₁₅-θ₁₆-G, where θ₁ is G, E, or D; θ₂ is I; θ₃ is Y or F; θ₄ is P; θ₅ is N, I, or R; θ₆ is D; θ₇ is G; θ₈ is Y or I; θ₉ is T; θ₁₄ is S; θ₁₅ is V; and θ₁₆ is K; and
a sequence selected from SEQ ID NOs: 181-239, or a variant thereof having three, two, or one amino acid substitutions(s).

8. The antibody of claim 7, wherein the Kabat CDR-H2 sequence is a sequence selected from SEQ ID NOs: 181-239.

9. The antibody of claim 1, wherein the antibody comprises a Kabat CDR-H1 sequence selected from:
a sequence defined by the consensus sequence ζ₁-ζ₂-ζ₃-ζ₄-ζ₅, where ζ₁ is N, K, T, R, H, Y, L, M, Q, or V; ζ₂ is Y, H, S, N, K, F, or Q; ζ₃ is S or Y; ζ₄ is I; and ζ₅ is H;
a sequence defined by the consensus sequence ζ₁-ζ₂-ζ₃-ζ₄-ζ₅, where ζ₁ is N or T; ζ₂ is Y, K, or Q; ζ₃ is S; ζ₄ is I; and ζ₅ is H; and
a sequence selected from SEQ ID NOs: 63-121, or a variant thereof having two or one amino acid substitutions.

10. The antibody of claim 9, wherein the Kabat CDR-H1 sequence is a sequence selected from SEQ ID NOs: 63-121.

11. The antibody of claim 1, wherein the antibody comprises a CDR-L3 sequence selected from SEQ ID NOs: 305-307, or a variant thereof having three, two, or one amino acid substitution(s).

12. The antibody of claim 1, wherein the antibody comprises a CDR-L2 sequence selected from a sequence selected from SEQ ID NOs: 302-304, or a variant thereof having two or one amino acid substitution(s).

13. The antibody of claim 1, wherein the antibody comprises a CDR-L1 sequence selected from a sequence selected from SEQ ID NOs: 299-301, or a variant thereof having three, two, or one amino acid substitution(s).

14. The antibody of claim 1, wherein the antibody comprises:
a V_{H} comprising: a CDR-H1 comprising one or more of SEQ ID NOs: 4 and 63; a CDR-H2 comprising one or more of SEQ ID NOs: 122 and 181; and a CDR-H3 comprising SEQ ID NO: 240;
a V_{H} comprising: a CDR-H1 comprising one or more of SEQ ID NOs: 5 and 64; a CDR-H2 comprising one or more of SEQ ID NOs: 123 and 182; and a CDR-H3 comprising SEQ ID NO: 241;
a V_{H} comprising: a CDR-H1 comprising one or more of SEQ ID NOs: 6 and 65; a CDR-H2 comprising one or more of SEQ ID NOs: 124 and 183; and a CDR-H3 comprising SEQ ID NO: 242;
a V_{H} comprising: a CDR-H1 comprising one or more of SEQ ID NOs: 7 and 66; a CDR-H2 comprising one or more of SEQ ID NOs: 125 and 184; and a CDR-H3 comprising SEQ ID NO: 243;
a V_{H} comprising: a CDR-H1 comprising one or more of SEQ ID NOs: 8 and 67; a CDR-H2 comprising one or more of SEQ ID NOs: 126 and 185; and a CDR-H3 comprising SEQ ID NO: 244;
a V_{H} comprising: a CDR-H1 comprising one or more of SEQ ID NOs: 9 and 68; a CDR-H2 comprising one or more of SEQ ID NOs: 127 and 186; and a CDR-H3 comprising SEQ ID NO: 245;
a V_{H} comprising: a CDR-H1 comprising one or more of SEQ ID NOs: 10 and 69; a CDR-H2 comprising one or more of SEQ ID NOs: 128 and 187; and a CDR-H3 comprising SEQ ID NO: 246;
a V_{H} comprising: a CDR-H1 comprising one or more of SEQ ID NOs: 11 and 70; a CDR-H2 comprising one or more of SEQ ID NOs: 129 and 188; and a CDR-H3 comprising SEQ ID NO: 247;
a V_{H} comprising: a CDR-H1 comprising one or more of SEQ ID NOs: 12 and 71; a CDR-H2 comprising one or more of SEQ ID NOs: 130 and 189; and a CDR-H3 comprising SEQ ID NO: 248;
a V_{H} comprising: a CDR-H1 comprising one or more of SEQ ID NOs: 13 and 72; a CDR-H2 comprising one or more of SEQ ID NOs: 131 and 190; and a CDR-H3 comprising SEQ ID NO: 249;
a V_{H} comprising: a CDR-H1 comprising one or more of SEQ ID NOs: 14 and 73; a CDR-H2 comprising one or more of SEQ ID NOs: 132 and 191; and a CDR-H3 comprising SEQ ID NO: 250;
a V_{H} comprising: a CDR-H1 comprising one or more of SEQ ID NOs: 15 and 74; a CDR-H2 comprising one or more of SEQ ID NOs: 133 and 192; and a CDR-H3 comprising SEQ ID NO: 251;
a V_{H} comprising: a CDR-H1 comprising one or more of SEQ ID NOs: 16 and 75; a CDR-H2 comprising one or more of SEQ ID NOs: 134 and 193; and a CDR-H3 comprising SEQ ID NO: 252;
a V_{H} comprising: a CDR-H1 comprising one or more of SEQ ID NOs: 17 and 76; a CDR-H2 comprising one or more of SEQ ID NOs: 135 and 194; and a CDR-H3 comprising SEQ ID NO: 253;
a V_{H} comprising: a CDR-H1 comprising one or more of SEQ ID NOs: 18 and 77; a CDR-H2 comprising one or more of SEQ ID NOs: 136 and 195; and a CDR-H3 comprising SEQ ID NO: 254;
a V_{H} comprising: a CDR-H1 comprising one or more of SEQ ID NOs: 19 and 78; a CDR-H2 comprising one or more of SEQ ID NOs: 137 and 196; and a CDR-H3 comprising SEQ ID NO: 255;
a V_{H} comprising: a CDR-H1 comprising one or more of SEQ ID NOs: 20 and 79; a CDR-H2 comprising one or more of SEQ ID NOs: 138 and 197; and a CDR-H3 comprising SEQ ID NO: 256;
a V_{H} comprising: a CDR-H1 comprising one or more of SEQ ID NOs: 21 and 80; a CDR-H2 comprising one or more of SEQ ID NOs: 139 and 198; and a CDR-H3 comprising SEQ ID NO: 257;
a V_{H} comprising: a CDR-H1 comprising one or more of SEQ ID NOs: 22 and 81; a CDR-H2 comprising one or more of SEQ ID NOs: 140 and 199; and a CDR-H3 comprising SEQ ID NO: 258;
a V_{H} comprising: a CDR-H1 comprising one or more of SEQ ID NOs: 23 and 82; a CDR-H2 comprising one or more of SEQ ID NOs: 141 and 200; and a CDR-H3 comprising SEQ ID NO: 259;
a V_{H} comprising: a CDR-H1 comprising one or more of SEQ ID NOs: 24 and 83; a CDR-H2 comprising one or more of SEQ ID NOs: 142 and 201; and a CDR-H3 comprising SEQ ID NO: 260;
a V_{H} comprising: a CDR-H1 comprising one or more of SEQ ID NOs: 25 and 84; a CDR-H2 comprising one or more of SEQ ID NOs: 143 and 202; and a CDR-H3 comprising SEQ ID NO: 261;
a V_{H} comprising: a CDR-H1 comprising one or more of SEQ ID NOs: 26 and 85; a CDR-H2 comprising one or more of SEQ ID NOs: 144 and 203; and a CDR-H3 comprising SEQ ID NO: 262;
a V_{H} comprising: a CDR-H1 comprising one or more of SEQ ID NOs: 27 and 86; a CDR-H2 comprising one or more of SEQ ID NOs: 145 and 204; and a CDR-H3 comprising SEQ ID NO: 263;
a V_{H} comprising: a CDR-H1 comprising one or more of SEQ ID NOs: 28 and 87; a CDR-H2 comprising one or more of SEQ ID NOs: 146 and 205; and a CDR-H3 comprising SEQ ID NO: 264;
a V_{H} comprising: a CDR-H1 comprising one or more of SEQ ID NOs: 29 and 88; a CDR-H2 comprising one or more of SEQ ID NOs: 147 and 206; and a CDR-H3 comprising SEQ ID NO: 265;
a V_{H} comprising: a CDR-H1 comprising one or more of SEQ ID NOs: 30 and 89; a CDR-H2 comprising one or more of SEQ ID NOs: 148 and 207; and a CDR-H3 comprising SEQ ID NO: 266;
a V_{H} comprising: a CDR-H1 comprising one or more of SEQ ID NOs: 31 and 90; a CDR-H2 comprising one or more of SEQ ID NOs: 149 and 208; and a CDR-H3 comprising SEQ ID NO: 267;
a V_{H} comprising: a CDR-H1 comprising one or more of SEQ ID NOs: 32 and 91; a CDR-H2 comprising one or more of SEQ ID NOs: 150 and 209; and a CDR-H3 comprising SEQ ID NO: 268;
a V_{H} comprising: a CDR-H1 comprising one or more of SEQ ID NOs: 33 and 92; a CDR-H2 comprising one or more of SEQ ID NOs: 151 and 210; and a CDR-H3 comprising SEQ ID NO: 269;
a V_{H} comprising: a CDR-H1 comprising one or more of SEQ ID NOs: 34 and 93; a CDR-H2 comprising one or more of SEQ ID NOs: 152 and 211; and a CDR-H3 comprising SEQ ID NO: 270;
a V_{H} comprising: a CDR-H1 comprising one or more of SEQ ID NOs: 35 and 94; a CDR-H2 comprising one or more of SEQ ID NOs: 153 and 212; and a CDR-H3 comprising SEQ ID NO: 271;
a V_{H} comprising: a CDR-H1 comprising one or more of SEQ ID NOs: 36 and 95; a CDR-H2 comprising one or more of SEQ ID NOs: 154 and 213; and a CDR-H3 comprising SEQ ID NO: 272;
a V_{H} comprising: a CDR-H1 comprising one or more of SEQ ID NOs: 37 and 96; a CDR-H2 comprising one or more of SEQ ID NOs: 155 and 214; and a CDR-H3 comprising SEQ ID NO: 273;
a V_{H} comprising: a CDR-H1 comprising one or more of SEQ ID NOs: 38 and 97; a CDR-H2 comprising one or more of SEQ ID NOs: 156 and 215; and a CDR-H3 comprising SEQ ID NO: 274;
a V_{H} comprising: a CDR-H1 comprising one or more of SEQ ID NOs: 39 and 98; a CDR-H2 comprising one or more of SEQ ID NOs: 157 and 216; and a CDR-H3 comprising SEQ ID NO: 275;
a V_{H} comprising: a CDR-H1 comprising one or more of SEQ ID NOs: 40 and 99; a CDR-H2 comprising one or more of SEQ ID NOs: 158 and 217; and a CDR-H3 comprising SEQ ID NO: 276;
a V_{H} comprising: a CDR-H1 comprising one or more of SEQ ID NOs: 41 and 100; a CDR-H2 comprising one or more of SEQ ID NOs: 159 and 218; and a CDR-H3 comprising SEQ ID NO: 277;
a V_{H} comprising: a CDR-H1 comprising one or more of SEQ ID NOs: 42 and 101; a CDR-H2 comprising one or more of SEQ ID NOs: 160 and 219; and a CDR-H3 comprising SEQ ID NO: 278;
a V_{H} comprising: a CDR-H1 comprising one or more of SEQ ID NOs: 43 and 102; a CDR-H2 comprising one or more of SEQ ID NOs: 161 and 220; and a CDR-H3 comprising SEQ ID NO: 279;
a V_{H} comprising: a CDR-H1 comprising one or more of SEQ ID NOs: 44 and 103; a CDR-H2 comprising one or more of SEQ ID NOs: 162 and 221; and a CDR-H3 comprising SEQ ID NO: 280;
a V_{H} comprising: a CDR-H1 comprising one or more of SEQ ID NOs: 45 and 104; a CDR-H2 comprising one or more of SEQ ID NOs: 163 and 222; and a CDR-H3 comprising SEQ ID NO: 281;
a V_{H} comprising: a CDR-H1 comprising one or more of SEQ ID NOs: 46 and 105; a CDR-H2 comprising one or more of SEQ ID NOs: 164 and 223; and a CDR-H3 comprising SEQ ID NO: 282;
a V_{H} comprising: a CDR-H1 comprising one or more of SEQ ID NOs: 47 and 106; a CDR-H2 comprising one or more of SEQ ID NOs: 165 and 224; and a CDR-H3 comprising SEQ ID NO: 283;
a V_{H} comprising: a CDR-H1 comprising one or more of SEQ ID NOs: 48 and 107; a CDR-H2 comprising one or more of SEQ ID NOs: 166 and 225; and a CDR-H3 comprising SEQ ID NO: 284;
a V_{H} comprising: a CDR-H1 comprising one or more of SEQ ID NOs: 49 and 108; a CDR-H2 comprising one or more of SEQ ID NOs: 167 and 226; and a CDR-H3 comprising SEQ ID NO: 285;
a V_{H} comprising: a CDR-H1 comprising one or more of SEQ ID NOs: 50 and 109; a CDR-H2 comprising one or more of SEQ ID NOs: 168 and 227; and a CDR-H3 comprising SEQ ID NO: 286;
a V_{H} comprising: a CDR-H1 comprising one or more of SEQ ID NOs: 51 and 110; a CDR-H2 comprising one or more of SEQ ID NOs: 169 and 228; and a CDR-H3 comprising SEQ ID NO: 287;
a V_{H} comprising: a CDR-H1 comprising one or more of SEQ ID NOs: 52 and 111; a CDR-H2 comprising one or more of SEQ ID NOs: 170 and 229; and a CDR-H3 comprising SEQ ID NO: 288;
a V_{H} comprising: a CDR-H1 comprising one or more of SEQ ID NOs: 53 and 112; a CDR-H2 comprising one or more of SEQ ID NOs: 171 and 230; and a CDR-H3 comprising SEQ ID NO: 289;
a V_{H} comprising: a CDR-H1 comprising one or more of SEQ ID NOs: 54 and 113; a CDR-H2 comprising one or more of SEQ ID NOs: 172 and 231; and a CDR-H3 comprising SEQ ID NO: 290;
a V_{H} comprising: a CDR-H1 comprising one or more of SEQ ID NOs: 55 and 114; a CDR-H2 comprising one or more of SEQ ID NOs: 173 and 232; and a CDR-H3 comprising SEQ ID NO: 291;
a V_{H} comprising: a CDR-H1 comprising one or more of SEQ ID NOs: 56 and 115; a CDR-H2 comprising one or more of SEQ ID NOs: 174 and 233; and a CDR-H3 comprising SEQ ID NO: 292;
a V_{H} comprising: a CDR-H1 comprising one or more of SEQ ID NOs: 57 and 116; a CDR-H2 comprising one or more of SEQ ID NOs: 175 and 234; and a CDR-H3 comprising SEQ ID NO: 293;
a V_{H} comprising: a CDR-H1 comprising one or more of SEQ ID NOs: 58 and 117; a CDR-H2 comprising one or more of SEQ ID NOs: 176 and 235; and a CDR-H3 comprising SEQ ID NO: 294;
a V_{H} comprising: a CDR-H1 comprising one or more of SEQ ID NOs: 59 and 118; a CDR-H2 comprising one or more of SEQ ID NOs: 177 and 236; and a CDR-H3 comprising SEQ ID NO: 295;
a V_{H} comprising: a CDR-H1 comprising one or more of SEQ ID NOs: 60 and 119; a CDR-H2 comprising one or more of SEQ ID NOs: 178 and 237; and a CDR-H3 comprising SEQ ID NO: 296;
a V_{H} comprising: a CDR-H1 comprising one or more of SEQ ID NOs: 61 and 120; a CDR-H2 comprising one or more of SEQ ID NOs: 179 and 238; and a CDR-H3 comprising SEQ ID NO: 297; or
a V_{H} comprising: a CDR-H1 comprising one or more of SEQ ID NOs: 62 and 121; a CDR-H2 comprising one or more of SEQ ID NOs: 180 and 239; and a CDR-H3 comprising SEQ ID NO: 298.

15. The antibody of claim 14, wherein the V_{H} is selected from SEQ ID NOs: 308-366, or a variant thereof having 20 or fewer amino acid substitutions.

16. The antibody of claim 14, wherein the antibody comprises:
a V_{L} comprising: a CDR-L1 comprising SEQ ID NO: 300; a CDR-L2 comprising SEQ ID NO: 303; and a CDR-L3 comprising SEQ ID NO: 306; or
a V_{L} comprising: a CDR-L1 comprising SEQ ID NO: 301; a CDR-L2 comprising SEQ ID NO: 304; and a CDR-L3 comprising SEQ ID NO: 307.

17. The antibody of claim 16 wherein the V_{L} sequence is selected from SEQ ID NOs: 368 and 369, or a variant thereof having 20 or fewer amino acid substitutions.

18. An antibody comprising a V_{H} region selected from SEQ ID NOs: 308-366, or a variant thereof having 20 or fewer amino acid substitutions, and a V_{L} region selected from SEQ ID NOs: 367-369, or a variant thereof having 20 or fewer amino acid substitutions.

19. The antibody of claim 18, wherein:
the V_{H} region is SEQ ID NO: 308, or the variant thereof, and the V_{L} region is SEQ ID NO: 367, or the variant thereof;
the V_{H} region is SEQ ID NO: 309, or the variant thereof, and the V_{L} region is SEQ ID NO: 367, or the variant thereof;
the V_{H} region is SEQ ID NO: 310, or the variant thereof, and the V_{L} region is SEQ ID NO: 367, or the variant thereof;
the V_{H} region is SEQ ID NO: 311, or the variant thereof, and the V_{L} region is SEQ ID NO: 367, or the variant thereof;
the V_{H} region is SEQ ID NO: 312, or the variant thereof, and the V_{L} region is SEQ ID NO: 367, or the variant thereof;
the V_{H} region is SEQ ID NO: 313, or the variant thereof, and the V_{L} region is SEQ ID NO: 367, or the variant thereof;
the V_{H} region is SEQ ID NO: 314, or the variant thereof, and the V_{L} region is SEQ ID NO: 367, or the variant thereof;
the V_{H} region is SEQ ID NO: 315, or the variant thereof, and the V_{L} region is SEQ ID NO: 367, or the variant thereof;
the V_{H} region is SEQ ID NO: 316, or the variant thereof, and the V_{L} region is SEQ ID NO: 367, or the variant thereof;
the V_{H} region is SEQ ID NO: 317, or the variant thereof, and the V_{L} region is SEQ ID NO: 367, or the variant thereof;
the V_{H} region is SEQ ID NO: 318, or the variant thereof, and the V_{L} region is SEQ ID NO: 367, or the variant thereof;
the V_{H} region is SEQ ID NO: 319, or the variant thereof, and the V_{L} region is SEQ ID NO: 367, or the variant thereof;
the V_{H} region is SEQ ID NO: 320, or the variant thereof, and the V_{L} region is SEQ ID NO: 367, or the variant thereof;
the V_{H} region is SEQ ID NO: 321, or the variant thereof, and the V_{L} region is SEQ ID NO: 367, or the variant thereof;
the V_{H} region is SEQ ID NO: 322, or the variant thereof, and the V_{L} region is SEQ ID NO: 367, or the variant thereof;
the V_{H} region is SEQ ID NO: 323, or the variant thereof, and the V_{L} region is SEQ ID NO: 367, or the variant thereof;
the V_{H} region is SEQ ID NO: 324, or the variant thereof, and the V_{L} region is SEQ ID NO: 367, or the variant thereof;
the V_{H} region is SEQ ID NO: 325, or the variant thereof, and the V_{L} region is SEQ ID NO: 367, or the variant thereof;
the V_{H} region is SEQ ID NO: 326, or the variant thereof, and the V_{L} region is SEQ ID NO: 367, or the variant thereof; or
the V_{H} region is SEQ ID NO: 327, or the variant thereof, and the V_{L} region is SEQ ID NO: 367, or the variant thereof;
the V_{H} region is SEQ ID NO: 328, or the variant thereof, and the V_{L} region is SEQ ID NO: 367, or the variant thereof;
the V_{H} region is SEQ ID NO: 329, or the variant thereof, and the V_{L} region is SEQ ID NO: 367, or the variant thereof;
the V_{H} region is SEQ ID NO: 330, or the variant thereof, and the V_{L} region is SEQ ID NO: 367, or the variant thereof;
the V_{H} region is SEQ ID NO: 331, or the variant thereof, and the V_{L} region is SEQ ID NO: 367, or the variant thereof;
the V_{H} region is SEQ ID NO: 332, or the variant thereof, and the V_{L} region is SEQ ID NO: 367, or the variant thereof;
the V_{H} region is SEQ ID NO: 333, or the variant thereof, and the V_{L} region is SEQ ID NO: 367, or the variant thereof;
the V_{H} region is SEQ ID NO: 334, or the variant thereof, and the V_{L} region is SEQ ID NO: 367, or the variant thereof;
the V_{H} region is SEQ ID NO: 335, or the variant thereof, and the V_{L} region is SEQ ID NO: 367, or the variant thereof;
the V_{H} region is SEQ ID NO: 336, or the variant thereof, and the V_{L} region is SEQ ID NO: 367, or the variant thereof;
the V_{H} region is SEQ ID NO: 337, or the variant thereof, and the V_{L} region is SEQ ID NO: 367, or the variant thereof;
the V_{H} region is SEQ ID NO: 338, or the variant thereof, and the V_{L} region is SEQ ID NO: 367, or the variant thereof;
the V_{H} region is SEQ ID NO: 339, or the variant thereof, and the V_{L} region is SEQ ID NO: 367, or the variant thereof;
the V_{H} region is SEQ ID NO: 340, or the variant thereof, and the V_{L} region is SEQ ID NO: 367, or the variant thereof;
the V_{H} region is SEQ ID NO: 341, or the variant thereof, and the V_{L} region is SEQ ID NO: 367, or the variant thereof;
the V_{H} region is SEQ ID NO: 342, or the variant thereof, and the V_{L} region is SEQ ID NO: 367, or the variant thereof;
the V_{H} region is SEQ ID NO: 343, or the variant thereof, and the V_{L} region is SEQ ID NO: 367, or the variant thereof;
the V_{H} region is SEQ ID NO: 344, or the variant thereof, and the V_{L} region is SEQ ID NO: 367, or the variant thereof;
the V_{H} region is SEQ ID NO: 345, or the variant thereof, and the V_{L} region is SEQ ID NO: 367, or the variant thereof;
the V_{H} region is SEQ ID NO: 346, or the variant thereof, and the V_{L} region is SEQ ID NO: 367, or the variant thereof;
the V_{H} region is SEQ ID NO: 347, or the variant thereof, and the V_{L} region is SEQ ID NO: 367, or the variant thereof;
the V_{H} region is SEQ ID NO: 348, or the variant thereof, and the V_{L} region is SEQ ID NO: 367, or the variant thereof;
the V_{H} region is SEQ ID NO: 349, or the variant thereof, and the V_{L} region is SEQ ID NO: 367, or the variant thereof;
the V_{H} region is SEQ ID NO: 350, or the variant thereof, and the V_{L} region is SEQ ID NO: 367, or the variant thereof;
the V_{H} region is SEQ ID NO: 351, or the variant thereof, and the V_{L} region is SEQ ID NO: 367, or the variant thereof;
the V_{H} region is SEQ ID NO: 352, or the variant thereof, and the V_{L} region is SEQ ID NO: 367, or the variant thereof;
the V_{H} region is SEQ ID NO: 353, or the variant thereof, and the V_{L} region is SEQ ID NO: 367, or the variant thereof;
the V_{H} region is SEQ ID NO: 354, or the variant thereof, and the V_{L} region is SEQ ID NO: 367, or the variant thereof;
the V_{H} region is SEQ ID NO: 355, or the variant thereof, and the V_{L} region is SEQ ID NO: 367, or the variant thereof;
the V_{H} region is SEQ ID NO: 356, or the variant thereof, and the V_{L} region is SEQ ID NO: 367, or the variant thereof;
the V_{H} region is SEQ ID NO: 357, or the variant thereof, and the V_{L} region is SEQ ID NO: 367, or the variant thereof;
the V_{H} region is SEQ ID NO: 358, or the variant thereof, and the V_{L} region is SEQ ID NO: 367, or the variant thereof;
the V_{H} region is SEQ ID NO: 359, or the variant thereof, and the V_{L} region is SEQ ID NO: 367, or the variant thereof;
the V_{H} region is SEQ ID NO: 360, or the variant thereof, and the V_{L} region is SEQ ID NO: 367, or the variant thereof;
the V_{H} region is SEQ ID NO: 361, or the variant thereof, and the V_{L} region is SEQ ID NO: 367, or the variant thereof;
the V_{H} region is SEQ ID NO: 362, or the variant thereof, and the V_{L} region is SEQ ID NO: 367, or the variant thereof;
the V_{H} region is SEQ ID NO: 363, or the variant thereof, and the V_{L} region is SEQ ID NO: 368, or the variant thereof;
the V_{H} region is SEQ ID NO: 364, or the variant thereof, and the V_{L} region is SEQ ID NO: 368, or the variant thereof;
the V_{H} region is SEQ ID NO: 365, or the variant thereof, and the V_{L} region is SEQ ID NO: 369, or the variant thereof; or
the V_{H} region is SEQ ID NO: 366, or the variant thereof, and the V_{L} region is SEQ ID NO: 369, or the variant thereof.

20. The antibody of claim 1, wherein the amino acid substitution is a conservative amino acid substitution.

21. The antibody of claim 1, wherein the antibody comprises at least one constant region domain.

22. The antibody of claim 21, wherein the constant region comprises a sequence selected from SEQ ID NOs: 370, 371, and 372.

23. The antibody of claim 1, wherein the antibody is a monoclonal antibody.

24. The antibody of claim 1, wherein the antibody is an IgA, an IgD, an IgE, an IgG, or an IgM.

25. The antibody of claim 1, wherein the antibody is humanized or human.

26. The antibody of claim 1, wherein the antibody is aglycosylated.

27. The antibody of claim 1, wherein the antibody is an antibody fragment.

28. The antibody of claim 27, wherein the antibody fragment is selected from an Fv fragment, a Fab fragment, a F(ab′)2 fragment, a Fab′ fragment, an scFv (sFv) fragment, and an scFv-Fc fragment.

29. The antibody of claim 28, wherein the antibody is an scFv fragment.

30. The antibody of claim 29, wherein the scFv fragment comprises a sequence selected from SEQ ID NO: 379 and SEQ ID NO: 380.

31. The antibody of claim 28, wherein the antibody is an scFv-Fc fragment.

32. The antibody of claim 31, wherein the scFv-Fc fragment comprises a sequence selected from SEQ ID NO: 381 and SEQ ID NO: 382.

33. The antibody of claim 1, wherein the antibody has a kₐ of about 2.90×10⁵ M⁻¹×sec⁻¹ to about 9.64×10⁹ M⁻¹×sec⁻¹ when associating with human folate receptor at a temperature of 25° C.

34. The antibody of claim 1, wherein the antibody has a k_{d} of about 2.28×10⁻⁴ sec⁻¹ to about 4.82×101 sec⁻¹ when dissociating from human folate receptor at a temperature of 25° C.

35. The antibody of claim 1, wherein the antibody has a K_{D} of about 2.26×10⁻¹¹ M to about 7.20×10⁻⁹ M when bound to human folate receptor at a temperature of 25° C.

36. The antibody of claim 1, wherein the antibody specifically binds cynomolgus folate receptor.

37. The antibody of claim 40, wherein the antibody has a K_{D} of about 0.19×10⁻⁹M to about 2.84×10⁻⁹ M when bound to cynomolgus folate receptor at a temperature of 25° C.

38. The antibody of claim 1, wherein the antibody specifically binds mouse folate receptor.

39. The antibody of claim 42, wherein the antibody has a K_{D} of about 0.5×10⁻⁹ M to about 9.07×10⁻⁸ M when bound to mouse folate receptor at a temperature of 25° C.

40. A kit comprising an antibody of claim 1, and instructions for use of the antibody.

41. The kit of claim 40, wherein the antibody is lyophilized.

42. The kit of claim 41, further comprising a fluid for reconstitution of the lyophilized antibody.

43. A polynucleotide encoding an antibody of claim 1.

44. A vector comprising the polynucleotide of claim 43.

45. A recombinant host cell comprising the vector of claim 44.

46. The host cell of claim 45, wherein the host cell is selected from a bacterial cell, a fungal cell, and a mammalian cell.

47. The host cell of claim 46, wherein the host cell is selected from an E. coli cell, a Saccharomyces cerevisiae cell, and a CHO cell.

48. A cell-free expression reaction comprising the vector of claim 44.

49. A pharmaceutical composition comprising the antibody of claim 1 and a pharmaceutically acceptable carrier.

50. A method of treating or preventing a disease or condition in a subject in need thereof, comprising administering to the subject an effective amount of an antibody of claim 1.

51. A method of diagnosing a disease or condition in a subject in need thereof, comprising administering to the subject an effective amount of an antibody of claim 1.

52. The method of claim 50, wherein the disease or condition is a cancer.

53. The method of claim 50, wherein the disease or condition is breast cancer.

54. The method of claim 50, wherein the disease or condition is triple-negative breast cancer (TNBC).

55. The method of claim 50, wherein the disease or condition is ovarian cancer.

56. The method of claim 50, wherein the disease or condition is lung cancer.

57. The method of claim 50, wherein the disease or condition is non-small cell lung cancer (NSCLC).

58. The method of claim 50, wherein the disease or condition is endometrial cancer.

59. A method of treating or preventing a disease or condition in a subject in need thereof, comprising administering to the subject an effective amount of a pharmaceutical composition of claim 49.

60. A method of diagnosing a disease or condition in a subject in need thereof, comprising administering to the subject an effective amount of a pharmaceutical composition of claim 49.


SEQUENCE LISTING (383 sequences)

SEQ ID NO: 1 - protein, 257 aa, Homo sapiens
  misc_feature (1)..(257): Human folate receptor alpha (hFOLR1)
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 257 aa, Macaca fascicularis
  misc_feature (1)..(257): Cynomolgus folate receptor alpha
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 3 - protein, 255 aa, Mus musculus
  misc_feature (1)..(255): Murine folate receptor alpha
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 4 - protein, 7 aa, Artificial Sequence
  Synthetic SRP1848-A01
GFNITRY

SEQ ID NO: 5 - protein, 7 aa, Artificial Sequence
  Synthetic SRP1848-A02
GFNISGF

SEQ ID NO: 6 - protein, 7 aa, Artificial Sequence
  Synthetic SRP1848-A04
GFNIDQS

SEQ ID NO: 7 - protein, 7 aa, Artificial Sequence
  Synthetic SRP1848-A06
GFNIGNS

SEQ ID NO: 8 - protein, 7 aa, Artificial Sequence
  Synthetic SRP1848-A07
GFNIGYH

SEQ ID NO: 9 - protein, 7 aa, Artificial Sequence
  Synthetic SRP1848-A08
GSNIRKH

SEQ ID NO: 10 - protein, 7 aa, Artificial Sequence
  Synthetic SRP1848-A09
GFNIRKQ

SEQ ID NO: 11 - protein, 7 aa, Artificial Sequence
  Synthetic SRP1848-A10
GFNIRKY

SEQ ID NO: 12 - protein, 7 aa, Artificial Sequence
  Synthetic SRP1848-B01
GFNIRNY

SEQ ID NO: 13 - protein, 7 aa, Artificial Sequence
  Synthetic SRP1848-B03
GFNISMK

SEQ ID NO: 14 - protein, 7 aa, Artificial Sequence
  Synthetic SRP1848-B04
SFNISNH

SEQ ID NO: 15 - protein, 7 aa, Artificial Sequence
  Synthetic SRP1848-B05
GFNISNY

SEQ ID NO: 16 - protein, 7 aa, Artificial Sequence
  Synthetic SRP1848-B06
GFNISNY

SEQ ID NO: 17 - protein, 7 aa, Artificial Sequence
  Synthetic SRP1848-B07
GFNISRF

SEQ ID NO: 18 - protein, 7 aa, Artificial Sequence
  Synthetic SRP1848-B09
GFNITNY

SEQ ID NO: 19 - protein, 7 aa, Artificial Sequence
  Synthetic SRP1848-B10
GFNTTTK

SEQ ID NO: 20 - protein, 7 aa, Artificial Sequence
  Synthetic SRP1848-B11
GFNIGNN

SEQ ID NO: 21 - protein, 7 aa, Artificial Sequence
  Synthetic SRP1848-C01
GFNIGNS

SEQ ID NO: 22 - protein, 7 aa, Artificial Sequence
  Synthetic SRP1848-C03
GFNIGVY

SEQ ID NO: 23 - protein, 7 aa, Artificial Sequence
  Synthetic SRP1848-C04
GFNIRHY

SEQ ID NO: 24 - protein, 7 aa, Artificial Sequence
  Synthetic SRP1848-C05
GFNIRKY

SEQ ID NO: 25 - protein, 7 aa, Artificial Sequence
  Synthetic SRP1848-C07
GFNIRKY

SEQ ID NO: 26 - protein, 7 aa, Artificial Sequence
  Synthetic SRP1848-C10
GFNIRTY

SEQ ID NO: 27 - protein, 7 aa, Artificial Sequence
  Synthetic SRP1848-D02
GFNISHN

SEQ ID NO: 28 - protein, 7 aa, Artificial Sequence
  Synthetic SRP1848-D03
GFNIRYF

SEQ ID NO: 29 - protein, 7 aa, Artificial Sequence
  Synthetic SRP1848-D04
GFNISHY

SEQ ID NO: 30 - protein, 7 aa, Artificial Sequence
  Synthetic SRP1848-D05
GFNISIS

SEQ ID NO: 31 - protein, 7 aa, Artificial Sequence
  Synthetic SRP1848-D07
GFNISKY

SEQ ID NO: 32 - protein, 7 aa, Artificial Sequence
  Synthetic SRP1848-D09
GFNISNY

SEQ ID NO: 33 - protein, 7 aa, Artificial Sequence
  Synthetic SRP1848-D10
GFNISRN

SEQ ID NO: 34 - protein, 7 aa, Artificial Sequence
  Synthetic SRP1848-E01
GFNITNK

SEQ ID NO: 35 - protein, 7 aa, Artificial Sequence
  Synthetic SRP1848-E02
GFNIGKY

SEQ ID NO: 36 - protein, 7 aa, Artificial Sequence
  Synthetic SRP1848-E03
GFNIGNY

SEQ ID NO: 37 - protein, 7 aa, Artificial Sequence
  Synthetic SRP1848-E05
GFNIGVY

SEQ ID NO: 38 - protein, 7 aa, Artificial Sequence
  Synthetic SRP1848-E06
GFNINRY

SEQ ID NO: 39 - protein, 7 aa, Artificial Sequence
  Synthetic SRP1848-E07
GFNIRKS

SEQ ID NO: 40 - protein, 7 aa, Artificial Sequence
  Synthetic SRP1848-F01
GFNIRTY

SEQ ID NO: 41 - protein, 7 aa, Artificial Sequence
  Synthetic SRP1848-F02
GFNIRTY

SEQ ID NO: 42 - protein, 7 aa, Artificial Sequence
  Synthetic SRP1848-F04
GFNISNY

SEQ ID NO: 43 - protein, 7 aa, Artificial Sequence
  Synthetic SRP1848-F05
GFNISKS

SEQ ID NO: 44 - protein, 7 aa, Artificial Sequence
  Synthetic SRP1848-F06
GFNISLS

SEQ ID NO: 45 - protein, 7 aa, Artificial Sequence
  Synthetic SRP1848-F07
GFNISNH

SEQ ID NO: 46 - protein, 7 aa, Artificial Sequence
  Synthetic SRP1848-F08
GFNISNH

SEQ ID NO: 47 - protein, 7 aa, Artificial Sequence
  Synthetic SRP1848-F09
GFNISNH

SEQ ID NO: 48 - protein, 7 aa, Artificial Sequence
  Synthetic SRP1848-F10
GFNISNN

SEQ ID NO: 49 - protein, 7 aa, Artificial Sequence
  Synthetic SRP1848-F11
GFNISNN

SEQ ID NO: 50 - protein, 7 aa, Artificial Sequence
  Synthetic SRP1848-G01
GFNISRH

SEQ ID NO: 51 - protein, 7 aa, Artificial Sequence
  Synthetic SRP1848-G03
GFNISTY

SEQ ID NO: 52 - protein, 7 aa, Artificial Sequence
  Synthetic SRP1848-G04
GFNIHST

SEQ ID NO: 53 - protein, 7 aa, Artificial Sequence
  Synthetic SRP1848-G06
GFNIRST

SEQ ID NO: 54 - protein, 7 aa, Artificial Sequence
  Synthetic SRP1848-G07
GFNIHST

SEQ ID NO: 55 - protein, 7 aa, Artificial Sequence
  Synthetic SRP1848-G09
GFNIRGT

SEQ ID NO: 56 - protein, 7 aa, Artificial Sequence
  Synthetic SRP1848-G10
GFNIRST

SEQ ID NO: 57 - protein, 7 aa, Artificial Sequence
  Synthetic SRP1848-G11
GFNISST

SEQ ID NO: 58 - protein, 7 aa, Artificial Sequence
  Synthetic SRP1848-H01
GFNIRTQ

SEQ ID NO: 59 - protein, 9 aa, Artificial Sequence
  Synthetic SRP2060-E10
GFSLSTFGM

SEQ ID NO: 60 - protein, 9 aa, Artificial Sequence
  Synthetic SRP2060-E05
GFSLSTFGM

SEQ ID NO: 61 - protein, 9 aa, Artificial Sequence
  Synthetic SRP2060-B01
GFSLSTFGM

SEQ ID NO: 62 - protein, 9 aa, Artificial Sequence
  Synthetic SRP2060-A06
GFSLSTFGM

SEQ ID NO: 63 - protein, 5 aa, Artificial Sequence
  Synthetic SRP1848-A01
RYSIH

SEQ ID NO: 64 - protein, 5 aa, Artificial Sequence
  Synthetic SRP1848-A02
GFRIH

SEQ ID NO: 65 - protein, 5 aa, Artificial Sequence
  Synthetic SRP1848-A04
QSSIH

SEQ ID NO: 66 - protein, 5 aa, Artificial Sequence
  Synthetic SRP1848-A06
NSYIH

SEQ ID NO: 67 - protein, 5 aa, Artificial Sequence
  Synthetic SRP1848-A07
YHSIH

SEQ ID NO: 68 - protein, 5 aa, Artificial Sequence
  Synthetic SRP1848-A08
KHSIH

SEQ ID NO: 69 - protein, 5 aa, Artificial Sequence
  Synthetic SRP1848-A09
KQSIH

SEQ ID NO: 70 - protein, 5 aa, Artificial Sequence
  Synthetic SRP1848-A10
KYSIH

SEQ ID NO: 71 - protein, 5 aa, Artificial Sequence
  Synthetic SRP1848-B01
NYSIH

SEQ ID NO: 72 - protein, 5 aa, Artificial Sequence
  Synthetic SRP1848-B03
MKYIH

SEQ ID NO: 73 - protein, 5 aa, Artificial Sequence
  Synthetic SRP1848-B04
NHSIH

SEQ ID NO: 74 - protein, 5 aa, Artificial Sequence
  Synthetic SRP1848-B05
NYYIH

SEQ ID NO: 75 - protein, 5 aa, Artificial Sequence
  Synthetic SRP1848-B06
NYYIH

SEQ ID NO: 76 - protein, 5 aa, Artificial Sequence
  Synthetic SRP1848-B07
RFYIH

SEQ ID NO: 77 - protein, 5 aa, Artificial Sequence
  Synthetic SRP1848-B09
NYYIH

SEQ ID NO: 78 - protein, 5 aa, Artificial Sequence
  Synthetic SRP1848-B10
TKSIH

SEQ ID NO: 79 - protein, 5 aa, Artificial Sequence
  Synthetic SRP1848-B11
NNSIH

SEQ ID NO: 80 - protein, 5 aa, Artificial Sequence
  Synthetic SRP1848-C01
NSYIH

SEQ ID NO: 81 - protein, 5 aa, Artificial Sequence
  Synthetic SRP1848-C03
VYSIH

SEQ ID NO: 82 - protein, 5 aa, Artificial Sequence
  Synthetic SRP1848-C04
HYSIH

SEQ ID NO: 83 - protein, 5 aa, Artificial Sequence
  Synthetic SRP1848-C05
KYSIH

SEQ ID NO: 84 - protein, 5 aa, Artificial Sequence
  Synthetic SRP1848-C07
KYSIH

SEQ ID NO: 85 - protein, 5 aa, Artificial Sequence
  Synthetic SRP1848-C10
TYYIH

SEQ ID NO: 86 - protein, 5 aa, Artificial Sequence
  Synthetic SRP1848-D02
HNYIH

SEQ ID NO: 87 - protein, 5 aa, Artificial Sequence
  Synthetic SRP1848-D03
YFSIH

SEQ ID NO: 88 - protein, 5 aa, Artificial Sequence
  Synthetic SRP1848-D04
HYSIH

SEQ ID NO: 89 - protein, 5 aa, Artificial Sequence
  Synthetic SRP1848-D05
ISYIH

SEQ ID NO: 90 - protein, 5 aa, Artificial Sequence
  Synthetic SRP1848-D07
KYYIH

SEQ ID NO: 91 - protein, 5 aa, Artificial Sequence
  Synthetic SRP1848-D09
NYYIH

SEQ ID NO: 92 - protein, 5 aa, Artificial Sequence
  Synthetic SRP1848-D10
RNSIH

SEQ ID NO: 93 - protein, 5 aa, Artificial Sequence
  Synthetic SRP1848-E01
NKYIH

SEQ ID NO: 94 - protein, 5 aa, Artificial Sequence
  Synthetic SRP1848-E02
KYSIH

SEQ ID NO: 95 - protein, 5 aa, Artificial Sequence
  Synthetic SRP1848-E03
NYYIH

SEQ ID NO: 96 - protein, 5 aa, Artificial Sequence
  Synthetic SRP1848-E05
VYYIH

SEQ ID NO: 97 - protein, 5 aa, Artificial Sequence
  Synthetic SRP1848-E06
RYYIH

SEQ ID NO: 98 - protein, 5 aa, Artificial Sequence
  Synthetic SRP1848-E07
KSSIH

SEQ ID NO: 99 - protein, 5 aa, Artificial Sequence
  Synthetic SRP1848-F01
TYSIH

SEQ ID NO: 100 - protein, 5 aa, Artificial Sequence
  Synthetic SRP1848-F02
TYSIH

SEQ ID NO: 101 - protein, 5 aa, Artificial Sequence
  Synthetic SRP1848-F04
NYSIH

SEQ ID NO: 102 - protein, 5 aa, Artificial Sequence
  Synthetic SRP1848-F05
KSSIH

SEQ ID NO: 103 - protein, 5 aa, Artificial Sequence
  Synthetic SRP1848-F06
LSYIH

SEQ ID NO: 104 - protein, 5 aa, Artificial Sequence
  Synthetic SRP1848-F07
NHSIH

SEQ ID NO: 105 - protein, 5 aa, Artificial Sequence
  Synthetic SRP1848-F08
NHSIH

SEQ ID NO: 106 - protein, 5 aa, Artificial Sequence
  Synthetic SRP1848-F09
NHYIH

SEQ ID NO: 107 - protein, 5 aa, Artificial Sequence
  Synthetic SRP1848-F10
NNSIH

SEQ ID NO: 108 - protein, 5 aa, Artificial Sequence
  Synthetic SRP1848-F11
NNYIH

SEQ ID NO: 109 - protein, 5 aa, Artificial Sequence
  Synthetic SRP1848-G01
RHSIH

SEQ ID NO: 110 - protein, 5 aa, Artificial Sequence
  Synthetic SRP1848-G03
TYYIH

SEQ ID NO: 111 - protein, 5 aa, Artificial Sequence
  Synthetic SRP1848-G04
STDIH

SEQ ID NO: 112 - protein, 5 aa, Artificial Sequence
  Synthetic SRP1848-G06
STDIH

SEQ ID NO: 113 - protein, 5 aa, Artificial Sequence
  Synthetic SRP1848-G07
STDIH

SEQ ID NO: 114 - protein, 5 aa, Artificial Sequence
  Synthetic SRP1848-G09
GTDIH

SEQ ID NO: 115 - protein, 5 aa, Artificial Sequence
  Synthetic SRP1848-G10
STDIH

SEQ ID NO: 116 - protein, 5 aa, Artificial Sequence
  Synthetic SRP1848-G11
STDIH

SEQ ID NO: 117 - protein, 5 aa, Artificial Sequence
  Synthetic SRP1848-H01
TQSIH

SEQ ID NO: 118 - protein, 7 aa, Artificial Sequence
  Synthetic SRP2060-E10
TFGMGVG

SEQ ID NO: 119 - protein, 7 aa, Artificial Sequence
  Synthetic SRP2060-E05
TFGMGVG

SEQ ID NO: 120 - protein, 7 aa, Artificial Sequence
  Synthetic SRP2060-B01
TFGMGVG

SEQ ID NO: 121 - protein, 7 aa, Artificial Sequence
  Synthetic SRP2060-A06
TFGMGVG

SEQ ID NO: 122 - protein, 6 aa, Artificial Sequence
  Synthetic SRP1848-A01
LPESGG

SEQ ID NO: 123 - protein, 6 aa, Artificial Sequence
  Synthetic SRP1848-A02
YPESGA

SEQ ID NO: 124 - protein, 6 aa, Artificial Sequence
  Synthetic SRP1848-A04
YPVDGT

SEQ ID NO: 125 - protein, 6 aa, Artificial Sequence
  Synthetic SRP1848-A06
TPIDGN

SEQ ID NO: 126 - protein, 6 aa, Artificial Sequence
  Synthetic SRP1848-A07
FPVDGT

SEQ ID NO: 127 - protein, 6 aa, Artificial Sequence
  Synthetic SRP1848-A08
YPNDGT

SEQ ID NO: 128 - protein, 6 aa, Artificial Sequence
  Synthetic SRP1848-A09
FPNDGT

SEQ ID NO: 129 - protein, 6 aa, Artificial Sequence
  Synthetic SRP1848-A10
FPIDDI

SEQ ID NO: 130 - protein, 6 aa, Artificial Sequence
  Synthetic SRP1848-B01
YPVDGI

SEQ ID NO: 131 - protein, 6 aa, Artificial Sequence
  Synthetic SRP1848-B03
TPIDGM

SEQ ID NO: 132 - protein, 6 aa, Artificial Sequence
  Synthetic SRP1848-B04
YPVDGI

SEQ ID NO: 133 - protein, 6 aa, Artificial Sequence
  Synthetic SRP1848-B05
SPIDGY

SEQ ID NO: 134 - protein, 6 aa, Artificial Sequence
  Synthetic SRP1848-B06
TPIDGY

SEQ ID NO: 135 - protein, 6 aa, Artificial Sequence
  Synthetic SRP1848-B07
SPYDGF

SEQ ID NO: 136 - protein, 6 aa, Artificial Sequence
  Synthetic SRP1848-B09
TPVDGY

SEQ ID NO: 137 - protein, 6 aa, Artificial Sequence
  Synthetic SRP1848-B10
YPRDGI

SEQ ID NO: 138 - protein, 6 aa, Artificial Sequence
  Synthetic SRP1848-B11
SPIDGF

SEQ ID NO: 139 - protein, 6 aa, Artificial Sequence
  Synthetic SRP1848-C01
TPNDGY

SEQ ID NO: 140 - protein, 6 aa, Artificial Sequence
  Synthetic SRP1848-C03
YPIDGN

SEQ ID NO: 141 - protein, 6 aa, Artificial Sequence
  Synthetic SRP1848-C04
YPGPGN

SEQ ID NO: 142 - protein, 6 aa, Artificial Sequence
  Synthetic SRP1848-C05
FPIDGI

SEQ ID NO: 143 - protein, 6 aa, Artificial Sequence
  Synthetic SRP1848-C07
FPIDGI

SEQ ID NO: 144 - protein, 6 aa, Artificial Sequence
  Synthetic SRP1848-C10
SPIDGY

SEQ ID NO: 145 - protein, 6 aa, Artificial Sequence
  Synthetic SRP1848-D02
TPQDGY

SEQ ID NO: 146 - protein, 6 aa, Artificial Sequence
  Synthetic SRP1848-D03
FPNDGS

SEQ ID NO: 147 - protein, 6 aa, Artificial Sequence
  Synthetic SRP1848-D04
YPRDGI

SEQ ID NO: 148 - protein, 6 aa, Artificial Sequence
  Synthetic SRP1848-D05
SPIDGY

SEQ ID NO: 149 - protein, 6 aa, Artificial Sequence
  Synthetic SRP1848-D07
SPNDGY

SEQ ID NO: 150 - protein, 6 aa, Artificial Sequence
  Synthetic SRP1848-D09
SPNDGY

SEQ ID NO: 151 - protein, 6 aa, Artificial Sequence
  Synthetic SRP1848-D10
SPNDGT

SEQ ID NO: 152 - protein, 6 aa, Artificial Sequence
  Synthetic SRP1848-E01
TPFDGF

SEQ ID NO: 153 - protein, 6 aa, Artificial Sequence
  Synthetic SRP1848-E02
YPNDGN

SEQ ID NO: 154 - protein, 6 aa, Artificial Sequence
  Synthetic SRP1848-E03
TPRDGF

SEQ ID NO: 155 - protein, 6 aa, Artificial Sequence
  Synthetic SRP1848-E05
TPNDGY

SEQ ID NO: 156 - protein, 6 aa, Artificial Sequence
  Synthetic SRP1848-E06
TPNDGY

SEQ ID NO: 157 - protein, 6 aa, Artificial Sequence
  Synthetic SRP1848-E07
FPYDGS

SEQ ID NO: 158 - protein, 6 aa, Artificial Sequence
  Synthetic SRP1848-F01
FPNDGT

SEQ ID NO: 159 - protein, 6 aa, Artificial Sequence
  Synthetic SRP1848-F02
FPNDGT

SEQ ID NO: 160 - protein, 6 aa, Artificial Sequence
  Synthetic SRP1848-F04
YPIDGI

SEQ ID NO: 161 - protein, 6 aa, Artificial Sequence
  Synthetic SRP1848-F05
YPNDGS

SEQ ID NO: 162 - protein, 6 aa, Artificial Sequence
  Synthetic SRP1848-F06
SPIDGN

SEQ ID NO: 163 - protein, 6 aa, Artificial Sequence
  Synthetic SRP1848-F07
YPNDGI

SEQ ID NO: 164 - protein, 6 aa, Artificial Sequence
  Synthetic SRP1848-F08
YPVDGI

SEQ ID NO: 165 - protein, 6 aa, Artificial Sequence
  Synthetic SRP1848-F09
SPLDGY

SEQ ID NO: 166 - protein, 6 aa, Artificial Sequence
  Synthetic SRP1848-F10
FPNDGY

SEQ ID NO: 167 - protein, 6 aa, Artificial Sequence
  Synthetic SRP1848-F11
TPIDGN

SEQ ID NO: 168 - protein, 6 aa, Artificial Sequence
  Synthetic SRP1848-G01
APNDGS

SEQ ID NO: 169 - protein, 6 aa, Artificial Sequence
  Synthetic SRP1848-G03
TPSDGF

SEQ ID NO: 170 - protein, 6 aa, Artificial Sequence
  Synthetic SRP1848-G04
TPAGGA

SEQ ID NO: 171 - protein, 6 aa, Artificial Sequence
  Synthetic SRP1848-G06
TPAGGA

SEQ ID NO: 172 - protein, 6 aa, Artificial Sequence
  Synthetic SRP1848-G07
TPAGGA

SEQ ID NO: 173 - protein, 6 aa, Artificial Sequence
  Synthetic SRP1848-G09
TPAGGA

SEQ ID NO: 174 - protein, 6 aa, Artificial Sequence
  Synthetic SRP1848-G10
TPAGGA

SEQ ID NO: 175 - protein, 6 aa, Artificial Sequence
  Synthetic SRP1848-G11
TPAGGA

SEQ ID NO: 176 - protein, 6 aa, Artificial Sequence
  Synthetic SRP1848-H01
FPIDGI

SEQ ID NO: 177 - protein, 5 aa, Artificial Sequence
  Synthetic SRP2060-E10
WWDDD

SEQ ID NO: 178 - protein, 5 aa, Artificial Sequence
  Synthetic SRP2060-E05
WWDDD

SEQ ID NO: 179 - protein, 5 aa, Artificial Sequence
  Synthetic SRP2060-B01
WWDDD

SEQ ID NO: 180 - protein, 5 aa, Artificial Sequence
  Synthetic SRP2060-A06
WWDDD

SEQ ID NO: 181 - protein, 17 aa, Artificial Sequence
  Synthetic SRP1848-A01
GILPESGGTSYADSVKG

SEQ ID NO: 182 - protein, 17 aa, Artificial Sequence
  Synthetic SRP1848-A02
GIYPESGATYYADSVKG

SEQ ID NO: 183 - protein, 17 aa, Artificial Sequence
  Synthetic SRP1848-A04
VIYPVDGTTDYADSVKG

SEQ ID NO: 184 - protein, 17 aa, Artificial Sequence
  Synthetic SRP1848-A06
GITPIDGNTDYADSVKG

SEQ ID NO: 185 - protein, 17 aa, Artificial Sequence
  Synthetic SRP1848-A07
EIFPVDGTTDYADSVKG

SEQ ID NO: 186 - protein, 17 aa, Artificial Sequence
  Synthetic SRP1848-A08
SIYPNDGTTDYADSVKG

SEQ ID NO: 187 - protein, 17 aa, Artificial Sequence
  Synthetic SRP1848-A09
SIFPNDGTTDYADSVKG

SEQ ID NO: 188 - protein, 17 aa, Artificial Sequence
  Synthetic SRP1848-A10
DIFPIDDITDYADSVKG

SEQ ID NO: 189 - protein, 17 aa, Artificial Sequence
  Synthetic SRP1848-B01
EIYPVDGITDYADSVKG

SEQ ID NO: 190 - protein, 17 aa, Artificial Sequence
  Synthetic SRP1848-B03
GITPIDGMTDYADSVKG

SEQ ID NO: 191 - protein, 17 aa, Artificial Sequence
  Synthetic SRP1848-B04
EIYPVDGITDYADSVKG

SEQ ID NO: 192 - protein, 17 aa, Artificial Sequence
  Synthetic SRP1848-B05
GISPIDGYTDYADSMKG

SEQ ID NO: 193 - protein, 17 aa, Artificial Sequence
  Synthetic SRP1848-B06
GITPIDGYTDYADSVKG

SEQ ID NO: 194 - protein, 17 aa, Artificial Sequence
  Synthetic SRP1848-B07
GISPYDGFTDYADSVKG

SEQ ID NO: 195 - protein, 17 aa, Artificial Sequence
  Synthetic SRP1848-B09
GITPVDGYTDYADRVKG

SEQ ID NO: 196 - protein, 17 aa, Artificial Sequence
  Synthetic SRP1848-B10
EIYPRDGITDYADSVKG

SEQ ID NO: 197 - protein, 17 aa, Artificial Sequence
  Synthetic SRP1848-B11
DISPIDGFTDYADSVKG

SEQ ID NO: 198 - protein, 17 aa, Artificial Sequence
  Synthetic SRP1848-C01
GVTPNDGYTDYADSVKG

SEQ ID NO: 199 - protein, 17 aa, Artificial Sequence
  Synthetic SRP1848-C03
EIYPIDGNTDYADSVKG

SEQ ID NO: 200 - protein, 17 aa, Artificial Sequence
  Synthetic SRP1848-C04
EIYPGPGNTDYADSVKG

SEQ ID NO: 201 - protein, 17 aa, Artificial Sequence
  Synthetic SRP1848-C05
DIFPIDGINDYADSVKG

SEQ ID NO: 202 - protein, 17 aa, Artificial Sequence
  Synthetic SRP1848-C07
DIFPIDGITDYADSVKG

SEQ ID NO: 203 - protein, 17 aa, Artificial Sequence
  Synthetic SRP1848-C10
GISPIDGYTDYADSMKG

SEQ ID NO: 204 - protein, 17 aa, Artificial Sequence
  Synthetic SRP1848-D02
GITPQDGYTDYADSVKG

SEQ ID NO: 205 - protein, 17 aa, Artificial Sequence
  Synthetic SRP1848-D03
DIFPNDGSTDYADSVKG

SEQ ID NO: 206 - protein, 17 aa, Artificial Sequence
  Synthetic SRP1848-D04
EIYPRDGITDYADSVKG

SEQ ID NO: 207 - protein, 17 aa, Artificial Sequence
  Synthetic SRP1848-D05
GISPIDGYTDYADSVKG

SEQ ID NO: 208 - protein, 17 aa, Artificial Sequence
  Synthetic SRP1848-D07
GISPNDGYTDYADSVKG

SEQ ID NO: 209 - protein, 17 aa, Artificial Sequence
  Synthetic SRP1848-D09
GISPNDGYTDYADSVKG

SEQ ID NO: 210 - protein, 17 aa, Artificial Sequence
  Synthetic SRP1848-D10
WISPNDGTTDYADSVKG

SEQ ID NO: 211 - protein, 17 aa, Artificial Sequence
  Synthetic SRP1848-E01
GITPFDGFTDYADSVKG

SEQ ID NO: 212 - protein, 17 aa, Artificial Sequence
  Synthetic SRP1848-E02
EIYPNDGNTDYADSVKG

SEQ ID NO: 213 - protein, 17 aa, Artificial Sequence
  Synthetic SRP1848-E03
GITPRDGFTDYADSVKG

SEQ ID NO: 214 - protein, 17 aa, Artificial Sequence
  Synthetic SRP1848-E05
GITPNDGYTDYADSVKG

SEQ ID NO: 215 - protein, 17 aa, Artificial Sequence
  Synthetic SRP1848-E06
GITPNDGYTDYADSVEG

SEQ ID NO: 216 - protein, 17 aa, Artificial Sequence
  Synthetic SRP1848-E07
EIFPYDGSTDYADNVKG

SEQ ID NO: 217 - protein, 17 aa, Artificial Sequence
  Synthetic SRP1848-F01
SIFPNDGTTDYADSVKG

SEQ ID NO: 218 - protein, 17 aa, Artificial Sequence
  Synthetic SRP1848-F02
SIFPNDGTTDYADSVKG

SEQ ID NO: 219 - protein, 17 aa, Artificial Sequence
  Synthetic SRP1848-F04
EIYPIDGITDYADSVKG

SEQ ID NO: 220 - protein, 17 aa, Artificial Sequence
  Synthetic SRP1848-F05
EIYPNDGSTDYADSVKG

SEQ ID NO: 221 - protein, 17 aa, Artificial Sequence
  Synthetic SRP1848-F06
GISPIDGNTDYADSVKG

SEQ ID NO: 222 - protein, 17 aa, Artificial Sequence
  Synthetic SRP1848-F07
EIYPNDGITDYADSVKG

SEQ ID NO: 223 - protein, 17 aa, Artificial Sequence
  Synthetic SRP1848-F08
EIYPVDGITDYADSVKG

SEQ ID NO: 224 - protein, 17 aa, Artificial Sequence
  Synthetic SRP1848-F09
GISPLDGYTDYADSVKG

SEQ ID NO: 225 - protein, 17 aa, Artificial Sequence
  Synthetic SRP1848-F10
SIFPNDGYTDYADSVKG

SEQ ID NO: 226 - protein, 17 aa, Artificial Sequence
  Synthetic SRP1848-F11
GITPIDGNTDYADSVKG

SEQ ID NO: 227 - protein, 17 aa, Artificial Sequence
  Synthetic SRP1848-G01
WIAPNDGSTDYADSVKG

SEQ ID NO: 228 - protein, 17 aa, Artificial Sequence
  Synthetic SRP1848-G03
GITPSDGFTDYADSVKG

SEQ ID NO: 229 - protein, 17 aa, Artificial Sequence
  Synthetic SRP1848-G04
YITPAGGATFYADSVKG

SEQ ID NO: 230 - protein, 17 aa, Artificial Sequence
  Synthetic SRP1848-G06
YITPAGGATYYADNVKG

SEQ ID NO: 231 - protein, 17 aa, Artificial Sequence
  Synthetic SRP1848-G07
YITPAGGATWYADSVKG

SEQ ID NO: 232 - protein, 17 aa, Artificial Sequence
  Synthetic SRP1848-G09
YITPAGGATFYADSVKG

SEQ ID NO: 233 - protein, 17 aa, Artificial Sequence
  Synthetic SRP1848-G10
YITPAGGATYYADSVKG

SEQ ID NO: 234 - protein, 17 aa, Artificial Sequence
  Synthetic SRP1848-G11
YITPAGGATWYADSVKG

SEQ ID NO: 235 - protein, 17 aa, Artificial Sequence
  Synthetic SRP1848-H01
DIFPIDGITDYADSVKG

SEQ ID NO: 236 - protein, 16 aa, Artificial Sequence
  Synthetic SRP2060-E10
HIWWDDDKYYHPALKG

SEQ ID NO: 237 - protein, 16 aa, Artificial Sequence
  Synthetic SRP2060-E05
HIWWDDDKYYHPALKG

SEQ ID NO: 238 - protein, 16 aa, Artificial Sequence
  Synthetic SRP2060-B01
HIWWDDDKYYHPALKG

SEQ ID NO: 239 - protein, 16 aa, Artificial Sequence
  Synthetic SRP2060-A06
HIWWDDDKYYYPALKG

SEQ ID NO: 240 - protein, 15 aa, Artificial Sequence
  Synthetic SRP1848-A01
HIYPWDWFSNYVLDY

SEQ ID NO: 241 - protein, 15 aa, Artificial Sequence
  Synthetic SRP1848-A02
HLYVWDWVLDHVLDY

SEQ ID NO: 242 - protein, 15 aa, Artificial Sequence
  Synthetic SRP1848-A04
GAWSWRSGYGYYIDY

SEQ ID NO: 243 - protein, 15 aa, Artificial Sequence
  Synthetic SRP1848-A06
GAWSWRSGYGYYIDY

SEQ ID NO: 244 - protein, 15 aa, Artificial Sequence
  Synthetic SRP1848-A07
GFWAWRSGYGYYLDY

SEQ ID NO: 245 - protein, 15 aa, Artificial Sequence
  Synthetic SRP1848-A08
GSWFWRAGYGYYLDY

SEQ ID NO: 246 - protein, 15 aa, Artificial Sequence
  Synthetic SRP1848-A09
GSWFWRSGYGYFLEY

SEQ ID NO: 247 - protein, 15 aa, Artificial Sequence
  Synthetic SRP1848-A10
GSWSWPSGHSYYLDY

SEQ ID NO: 248 - protein, 15 aa, Artificial Sequence
  Synthetic SRP1848-B01
GFWSWPSGYSYFLDY

SEQ ID NO: 249 - protein, 15 aa, Artificial Sequence
  Synthetic SRP1848-B03
GSWSWPSGYSYYLDY

SEQ ID NO: 250 - protein, 15 aa, Artificial Sequence
  Synthetic SRP1848-B04
GRYSWRAGYSYYLDY

SEQ ID NO: 251 - protein, 15 aa, Artificial Sequence
  Synthetic SRP1848-B05
GSWFWQSGYGYYLDY

SEQ ID NO: 252 - protein, 15 aa, Artificial Sequence
  Synthetic SRP1848-B06
GFWSWPSGYGYYQDY

SEQ ID NO: 253 - protein, 15 aa, Artificial Sequence
  Synthetic SRP1848-B07
GSWSWPAGYGYYQDY

SEQ ID NO: 254 - protein, 15 aa, Artificial Sequence
  Synthetic SRP1848-B09
GAWSWRSGYGYYMDY

SEQ ID NO: 255 - protein, 15 aa, Artificial Sequence
  Synthetic SRP1848-B10
GGWHWRSGYSYYLDY

SEQ ID NO: 256 - protein, 15 aa, Artificial Sequence
  Synthetic SRP1848-B11
GSWSWRAGYGYYLDY

SEQ ID NO: 257 - protein, 15 aa, Artificial Sequence
  Synthetic SRP1848-C01
GSWFWRAGYGYYLDY

SEQ ID NO: 258 - protein, 15 aa, Artificial Sequence
  Synthetic SRP1848-C03
GSWAWRSGYSYYLDY

SEQ ID NO: 259 - protein, 15 aa, Artificial Sequence
  Synthetic SRP1848-C04
GSLSWRAGYGYYLDY

SEQ ID NO: 260 - protein, 15 aa, Artificial Sequence
  Synthetic SRP1848-C05
GSWSWKAGYGYYLDY

SEQ ID NO: 261 - protein, 15 aa, Artificial Sequence
  Synthetic SRP1848-C07
GSWSWPAGYGYYQDY

SEQ ID NO: 262 - protein, 15 aa, Artificial Sequence
  Synthetic SRP1848-C10
GSWSWPAGYGYYLDY

SEQ ID NO: 263 - protein, 15 aa, Artificial Sequence
  Synthetic SRP1848-D02
GAWSWRAGYGYYLDY

SEQ ID NO: 264 - protein, 15 aa, Artificial Sequence
  Synthetic SRP1848-D03
GHWSWPSGYWYYLDY

SEQ ID NO: 265 - protein, 15 aa, Artificial Sequence
  Synthetic SRP1848-D04
GYWFWRSGYGYYLDY

SEQ ID NO: 266 - protein, 15 aa, Artificial Sequence
  Synthetic SRP1848-D05
GSWSWRAGYGYYLDY

SEQ ID NO: 267 - protein, 15 aa, Artificial Sequence
  Synthetic SRP1848-D07
GFWAWRSGYGYYLDY

SEQ ID NO: 268 - protein, 15 aa, Artificial Sequence
  Synthetic SRP1848-D09
GSWSWRHGYGYYLDY

SEQ ID NO: 269 - protein, 15 aa, Artificial Sequence
  Synthetic SRP1848-D10
GAWSWRSGYGYYIDY

SEQ ID NO: 270 - protein, 15 aa, Artificial Sequence
  Synthetic SRP1848-E01
GSWSWPAGYGYYQDY

SEQ ID NO: 271 - protein, 15 aa, Artificial Sequence
  Synthetic SRP1848-E02
GSWSWRSGYGYYLDY

SEQ ID NO: 272 - protein, 15 aa, Artificial Sequence
  Synthetic SRP1848-E03
GSWSWPAGHSYYLDY

SEQ ID NO: 273 - protein, 15 aa, Artificial Sequence
  Synthetic SRP1848-E05
GFWAWRSGYGYYLDY

SEQ ID NO: 274 - protein, 15 aa, Artificial Sequence
  Synthetic SRP1848-E06
GTWSWPSGHSYYLDY

SEQ ID NO: 275 - protein, 15 aa, Artificial Sequence
  Synthetic SRP1848-E07
GAWSWRSGYGYYIDY

SEQ ID NO: 276 - protein, 15 aa, Artificial Sequence
  Synthetic SRP1848-F01
GSWAWRAGYSYYLDY

SEQ ID NO: 277 - protein, 15 aa, Artificial Sequence
  Synthetic SRP1848-F02
GSWSWQAGYGYYLDY

SEQ ID NO: 278 - protein, 15 aa, Artificial Sequence
  Synthetic SRP1848-F04
GSWFWRSGYGYYLDY

SEQ ID NO: 279 - protein, 15 aa, Artificial Sequence
  Synthetic SRP1848-F05
GSWAWRSGYSYFLDY

SEQ ID NO: 280 - protein, 15 aa, Artificial Sequence
  Synthetic SRP1848-F06
GFWAWRSGYGYYLDY

SEQ ID NO: 281 - protein, 15 aa, Artificial Sequence
  Synthetic SRP1848-F07
GSWDWRSGYSYYLDY

SEQ ID NO: 282 - protein, 15 aa, Artificial Sequence
  Synthetic SRP1848-F08
GSWYWQSGYSYYLDY

SEQ ID NO: 283 - protein, 15 aa, Artificial Sequence
  Synthetic SRP1848-F09
GAWSWRSGYGYYIDY

SEQ ID NO: 284 - protein, 15 aa, Artificial Sequence
  Synthetic SRP1848-F10
GSWFWRSGYGYYLDY

SEQ ID NO: 285 - protein, 15 aa, Artificial Sequence
  Synthetic SRP1848-F11
GSWYWRAGYGYYLDY

SEQ ID NO: 286 - protein, 15 aa, Artificial Sequence
  Synthetic SRP1848-G01
GSWAWRSGYSYFLDY

SEQ ID NO: 287 - protein, 15 aa, Artificial Sequence
  Synthetic SRP1848-G03
GSWSWPSGHGYFLDY

SEQ ID NO: 288 - protein, 11 aa, Artificial Sequence
  Synthetic SRP1848-G04
YPYWFAGYMDY

SEQ ID NO: 289 - protein, 11 aa, Artificial Sequence
  Synthetic SRP1848-G06
QPYWFAGYMDY

SEQ ID NO: 290 - protein, 11 aa, Artificial Sequence
  Synthetic SRP1848-G07
YPFWFAGYMDY

SEQ ID NO: 291 - protein, 11 aa, Artificial Sequence
  Synthetic SRP1848-G09
HEYWFSGYMDY

SEQ ID NO: 292 - protein, 11 aa, Artificial Sequence
  Synthetic SRP1848-G10
YPYWFAGYIDY

SEQ ID NO: 293 - protein, 11 aa, Artificial Sequence
  Synthetic SRP1848-G11
YPYWFSGYMDY

SEQ ID NO: 294 - protein, 15 aa, Artificial Sequence
  Synthetic SRP1848-H01
GSWSWPSGMDYYLDY

SEQ ID NO: 295 - protein, 16 aa, Artificial Sequence
  Synthetic SRP2060-E10
NHFPHYYGSSHWYFNV

SEQ ID NO: 296 - protein, 16 aa, Artificial Sequence
  Synthetic SRP2060-E05
NHFPHYYGSSHWYFNV

SEQ ID NO: 297 - protein, 16 aa, Artificial Sequence
  Synthetic SRP2060-B01
NHFPHYYGSSHWYFNV

SEQ ID NO: 298 - protein, 16 aa, Artificial Sequence
  Synthetic SRP2060-A06
NHFPHYYGSSHWYFDV

SEQ ID NO: 299 - protein, 11 aa, Artificial Sequence
  Synthetic trastuzumab
RASQDVNTAVA

SEQ ID NO: 300 - protein, 11 aa, Artificial Sequence
  Synthetic H6D1-LC4
KASQDINSYLS

SEQ ID NO: 301 - protein, 11 aa, Artificial Sequence
  Synthetic H6D1-LC5
KASQDINSYLS

SEQ ID NO: 302 - protein, 7 aa, Artificial Sequence
  Synthetic trastuzumab
SASFLYS

SEQ ID NO: 303 - protein, 7 aa, Artificial Sequence
  Synthetic H6D1-LC4
RANRLVD

SEQ ID NO: 304 - protein, 7 aa, Artificial Sequence
  Synthetic H6D1-LC5
RANRLVD

SEQ ID NO: 305 - protein, 9 aa, Artificial Sequence
  Synthetic trastuzumab
QQHYTTPPT

SEQ ID NO: 306 - protein, 9 aa, Artificial Sequence
  Synthetic H6D1-LC4
LQYDEFPYT

SEQ ID NO: 307 - protein, 9 aa, Artificial Sequence
  Synthetic H6D1-LC5
LQYDEFPYT

SEQ ID NO: 308 - protein, 124 aa, Artificial Sequence
  Synthetic SRP1848-A01
EVQLVESGGGLVQPGGSLRLSCAASGFNITRYSIHWVRQAPGKGLEWVAGILPESGGTSYADSVKGRFTISADTSKNTAYLQMNSLRAEDTAVYYCARHIYPWDWFSNYVLDYWGQGTLVTVSS

SEQ ID NO: 309 - protein, 124 aa, Artificial Sequence
  Synthetic SRP1848-A02
EVQLVESGGGLVQPGGSLRLSCAASGFNISGFRIHWVRQAPGKGLEWVAGIYPESGATYYADSVKGRFTISADTSKNTAYLQMNSLRAEDTAVYYCARHLYVWDWVLDHVLDYWGQGTLVTVSS

SEQ ID NO: 310 - protein, 124 aa, Artificial Sequence
  Synthetic SRP1848-A04
EVQLVESGGGLVQPGGSLRLSCAASGFNIDQSSIHWVRQAPGKGLEWVGVIYPVDGTTDYADSVKGRFTISADTSKNTAYLQMNSLRAEDTAVYYCARGAWSWRSGYGYYIDYWGQGTLVTVSS

SEQ ID NO: 311 - protein, 124 aa, Artificial Sequence
  Synthetic SRP1848-A06
EVQLVESGGGLVQPGGSLRLSCAASGFNIGNSYIHWVRQAPGKGLEWVGGITPIDGNTDYADSVKGRFTISADTSKNTAYLQMNSLRAEDTAVYYCARGAWSWRSGYGYYIDYWGQGTLVTVSS

SEQ ID NO: 312 - protein, 124 aa, Artificial Sequence
  Synthetic SRP1848-A07
EVQLVESGGGLVQPGGSLRLSCAASGFNIGYHSIHWVRQAPGKGLEWVGEIFPVDGTTDYADSVKGRFTISADTSKNTAYLHMNSLRAEDTAVYYCARGFWAWRSGYGYYLDYWGQGTLVTVSS

SEQ ID NO: 313 - protein, 124 aa, Artificial Sequence
  Synthetic SRP1848-A08
EVQLVESGGGLVQPGGSLRLSCAASGSNIRKHSIHWVRQAPGKGLEWVGSIYPNDGTTDYADSVKGRFTISADTSKNTAYLQMNSLRAEDTAVYYCARGSWFWRAGYGYYLDYWGQGTLVTVSS

SEQ ID NO: 314 - protein, 124 aa, Artificial Sequence
  Synthetic SRP1848-A09
EVQLVESGGGLVQPGGSLRLSCAASGFNIRKQSIHWVRQAPGKGLEWVGSIFPNDGTTDYADSVKGRFTISADTSKNTAYLQVNSLRAEDTAVYYCARGSWFWRSGYGYFLEYWGQGTLVTVSS

SEQ ID NO: 315 - protein, 124 aa, Artificial Sequence
  Synthetic SRP1848-A10
EVQLVESGGGLVQPGGSLRLSCAASGFNIRKYSIHWARQAPGKGLEWVGDIFPIDDITDYADSVKGRFTISADTSKNTAYLQMNSLRAEDTAVYYCARGSWSWPSGHSYYLDYWGQGTLVTVSS

SEQ ID NO: 316 - protein, 124 aa, Artificial Sequence
  Synthetic SRP1848-B01
EVQLVESGGGLVQPGGSLRLSCAASGFNIRNYSIHWVRQAPGKGLEWVGEIYPVDGITDYADSVKGRFTISADTSKNTAYLQMNSLRAEDTAVYYCARGFWSWPSGYSYFLDYWGQGTLVTVSS

SEQ ID NO: 317 - protein, 124 aa, Artificial Sequence
  Synthetic SRP1848-B03
EVQLVESGGGLVQPGGSLRLSCAASGFNISMKYIHWVRQAPGKGLEWVGGITPIDGMTDYADSVKGRFTISADTSKNTAYLQMNSLRAEDTAVYYCARGSWSWPSGYSYYLDYWGQGTLVTVSS

SEQ ID NO: 318 - protein, 124 aa, Artificial Sequence
  Synthetic SRP1848-B04
EVQLVESGGGLVQPGGSLRLSCAASSFNISNHSIHWVRQAPGKGLEWVGEIYPVDGITDYADSVKGRFTISADTSKNTAYLQMNSLRAEDTAVYYCARGRYSWRAGYSYYLDYWGQGTLVTVSS

SEQ ID NO: 319 - protein, 124 aa, Artificial Sequence
  Synthetic SRP1848-B05
EVQLVESGGGLVQPGGSLRLSCAASGFNISNYYIHWVRQAPGKGLEWVGGISPIDGYTDYADSMKGRFTISADTSKNTAYLQMSSLRAEDTAVYYCARGSWFWQSGYGYYLDYWGQGTLVTVSS

SEQ ID NO: 320 - protein, 124 aa, Artificial Sequence
  Synthetic SRP1848-B06
EVQLVESGGGLVQPGGSLRLSCAASGFNISNYYIHWVRQAPGKGLEWVGGITPIDGYTDYADSVKGRFTISADTSKNTAYLQMNSLRAEDTAVYYCARGFWSWPSGYGYYQDYWGQGTLVTVSS

SEQ ID NO: 321 - protein, 124 aa, Artificial Sequence
  Synthetic SRP1848-B07
EVQLVESGGGLVQPGGSLRLSCAASGFNISRFYIHWVRQAPGKGLEWVGGISPYDGFTDYADSVKGRFTISADTSKNTAYLQMNSLRAEDTAVYYCARGSWSWPAGYGYYQDYWGQGTLVTVSS

SEQ ID NO: 322 - protein, 124 aa, Artificial Sequence
  Synthetic SRP1848-B09
EVQLVESGGGLVQPGGSLRLSCAAGGFNITNYYIHWVRQAPGKGLEWVGGITPVDGYTDYADRVKGRFTISADTSKNTAYLQMNSLRAEDTAVYYCARGAWSWRSGYGYYMDYWGQGTLVTVSS

SEQ ID NO: 323 - protein, 124 aa, Artificial Sequence
  Synthetic SRP1848-B10
EVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 324 - protein, 124 aa, Artificial Sequence
  Synthetic SRP1848-B11
EVQLVESGGGLVQPGGSLRLSCAASGFNIGNNSIHWVRQAPGKGLEWVGDISPIDGFTDYADSVKGRFTISADTSKNTAYLQMNSLRAEDTAVYYCARGSWSWRAGYGYYLDYWGQGTLVTVSS

SEQ ID NO: 325 - protein, 124 aa, Artificial Sequence
  Synthetic SRP1848-C01
EVQLVESGGGLVQPGGSLRLSCAASGFNIGNSYIHWVRQAPGKGLEWVGGVTPNDGYTDYADSVKGRFTISADTSKNTTYLQMNSLRAEDTAVYYCARGSWFWRAGYGYYLDYWGQGALVTVSS

SEQ ID NO: 326 - protein, 124 aa, Artificial Sequence
  Synthetic SRP1848-C03
EVQLVESGGGLVQPGGSLRLSCAASGFNIGVYSIHWVRQAPGKGLEWVGEIYPIDGNTDYADSVKGRFTISADTSKNTAYLQMNSLRAEDTAVYYCARGSWAWRSGYSYYLDYWGQGTLVTVSS

SEQ ID NO: 327 - protein, 124 aa, Artificial Sequence
  Synthetic SRP1848-C04
EVQLVESGGGLVQPGGSLRLSCAASGFNIRHYSIHWVRQAPGKGLEWVGEIYPGPGNTDYADSVKGRFTISADTSKNTAYLQMNSLRAEDTAVYYCARGSLSWRAGYGYYLDYWGQGTLVTVSS

SEQ ID NO: 328 - protein, 124 aa, Artificial Sequence
  Synthetic SRP1848-C05
EVQLVESGGGLVQPGGSLRLSCAASGFNIRKYSIHWVRQAPGKGLEWVGDIFPIDGINDYADSVKGRFTISADTSKNTAYLQMNSLRAEDTAVYYCARGSWSWKAGYGYYLDYWGQGTLVTVSS

SEQ ID NO: 329 - protein, 124 aa, Artificial Sequence
  Synthetic SRP1848-C07
EVQLVESGGGLVQPGGSLRLSCAASGFNIRKYSIHWVRQAPGKGLEWVGDIFPIDGITDYADSMKGRFTISADTSKNTAYLQMNSLRAEDTAVYYCARGSWSWPAGYGYYQDYWGQGTLVTVSS

SEQ ID NO: 330 - protein, 124 aa, Artificial Sequence
  Synthetic SRP1848-C10
EVQLVESGGGLVQPGGSLRLSCAASGFNIRTYYIHWVRQAPGKGLEWVGGISPIDGYTDYADSVKGRFTISADTSKNTAYLQMNSLRAEDTAVYYCARGSWSWPAGYGYYLDYWGQGTLVTVSS

SEQ ID NO: 331 - protein, 124 aa, Artificial Sequence
  Synthetic SRP1848-D02
EVQLVESGGGLVQPGGSLRLSCAASGFNISHNYIHWVRQAPGKGLEWVGGITPQDGYTDYADSVKGRFTISADTSKNTAYLQMNRLRAEDTAVYYCARGAWSWRAGYGYYLDYWGQGTLVTVSS

SEQ ID NO: 332 - protein, 124 aa, Artificial Sequence
  Synthetic SRP1848-D03
EVQLVESGGGVVQPGGSLRLSCAASGFNIRYFSIHWVRQAPGKGLEWVGDIFPNDGSTDYADSVKGRFTISADTSKNTAYLQMNSLRAEETAVYYCARGHWSWPSGYWYYLDYWGQGTLVTVSS

SEQ ID NO: 333 - protein, 124 aa, Artificial Sequence
  Synthetic SRP1848-D04
EVQLVESGGGLVQPGGSLRLSCAASGFNISHYSIHWVRQAPGKGLEWVGEIYPRDGITDYADSVKGRFTISADTSKNTAYLQMNSLSAEDTAVYYCARGYWFWRSGYGYYLDYWGQGTLVTVSS

SEQ ID NO: 334 - protein, 124 aa, Artificial Sequence
  Synthetic SRP1848-D05
EVQLVESGGGLVQPGGSLRLSCAASGFNISISYIHWVRQAPGKGLEWVGGISPIDGYTDYADSVKGRFTISADTSKNTAYLQMNSLRAEDTAVYYCARGSWSWRAGYGYYLDYWGQGTLVTVSS

SEQ ID NO: 335 - protein, 124 aa, Artificial Sequence
  Synthetic SRP1848-D07
EVQLVESGGGLVQPGGSLRLSCAASGFNISKYYIHWVRQAPGKGLEWVGGISPNDGYTDYADSVKGRFAISADTSKNTAYLQMNSLRAEDTAVYYCARGFWAWRSGYGYYLDYWGQGTLVTVSS

SEQ ID NO: 336 - protein, 124 aa, Artificial Sequence
  Synthetic SRP1848-D09
EVQLVESGGGLVQPGGSLRLSCAASGFNISNYYIHWVRQAPGKGLEWVGGISPNDGYTDYADSVKGRFTISADTSKNTAYLQMNSLRAEDTAVYYCARGSWSWRHGYGYYLDYWGQGTLVTVSS

SEQ ID NO: 337 - protein, 124 aa, Artificial Sequence
  Synthetic SRP1848-D10
EVQLVESGGGLVQPGGSLRLSCAASGFNISRNSIHWVRQAPGKGLEWVGWISPNDGTTDYADSVKGRFTISADGSKNTAYLQMNSLRAEDTAVYYCARGAWSWRSGYGYYIDYWGQGTLVTVSS

SEQ ID NO: 338 - protein, 124 aa, Artificial Sequence
  Synthetic SRP1848-E01
EVQLVESGGGLVQPGGSLRLSCAASGFNITNKYIHWVRQAPGKGLEWVGGITPFDGFTDYADSVKGRFTISADTSKNTAYLQMNSLRAEDTAVYYCARGSWSWPAGYGYYQDYWGQGTLVTVSS

SEQ ID NO: 339 - protein, 124 aa, Artificial Sequence
  Synthetic SRP1848-E02
EVQLVESGGGLVQPGGSLRLSCAASGFNIGKYSIHWVRQAPGKGLEWVGEIYPNDGNTDYADSVKGRFTISADTSKNTAYLQMNSLRAEDTAVYYCARGSWSWRSGYGYYLDYWGQGTLVTVSS

SEQ ID NO: 340 - protein, 124 aa, Artificial Sequence
  Synthetic SRP1848-E03
EVQLVESGGGLAQPGGSLRLSCAASGFNIGNYYIHWVRQAPGKGLEWVGGITPRDGFTDYADSVKGRFTISADTSKNTAYLQVNSLRAEDTAVYYCARGSWSWPAGHSYYLDYWGQGTLVTVSS

SEQ ID NO: 341 - protein, 124 aa, Artificial Sequence
  Synthetic SRP1848-E05
EVQLVESGGGLVQPGGSLRVSCAASGFNIGVYYIHWVRQAPGKGLEWVGGITPNDGYTDYADSVKGRFTISADTSKNTAYLQMNSLRAEDTAVYYCARGFWAWRSGYGYYLDYWGQGTLVTVSS

SEQ ID NO: 342 - protein, 124 aa, Artificial Sequence
  Synthetic SRP1848-E06
EVQLVESGGGLVQPSGSLRLSCAASGFNINRYYIHWVRQAPGKGLEWVGGITPNDGYTDYADSVEGRFTTSADTSKNTAYLQMNSLRAEDTAVYYCARGTWSWPSGHSYYLDYWGQGTLVTVSS

SEQ ID NO: 343 - protein, 124 aa, Artificial Sequence
  Synthetic SRP1848-E07
EVQLVESGGGLVQPGGSLRLSCAASGFNIRKSSIHWVRQAPGKGLEWVGEIFPYDGSTDYADNVKGRFTISADTSKNTAYLQMNSLRAEDTAVYYCARGAWSWRSGYGYYIDYWGQGTLVTVSS

SEQ ID NO: 344 - protein, 124 aa, Artificial Sequence
  Synthetic SRP1848-F01
EVQLVESGGGLVQPGGSLRLSCAASGFNIRTYSIHWVRQAPGKGLEWVGSIFPNDGTTDYADSVKGRFTISADTSKNTAYLQMNSLRAEDTAVYYCARGSWAWRAGYSYYLDYWGQGTLVTVSS

SEQ ID NO: 345 - protein, 124 aa, Artificial Sequence
  Synthetic SRP1848-F02
EVQLVESGGGLVQPGGSLRLSCAASGFNIRTYSIHWVRQAPGKGLEWVGSIFPNDGTTDYADSVKGRLTISADTSKNTAYLQMNSLRAEDTAVYYCARGSWSWQAGYGYYLDYWGQGTLVTVSS

SEQ ID NO: 346 - protein, 124 aa, Artificial Sequence
  Synthetic SRP1848-F04
EVQLVESGGGLVQPGGSLRLSCAASGFNISNYSIHWVRQAPGKGLEWVGEIYPIDGITDYADSVKGRFTISADTSKNTAYLQMNSLRAEDTAVYYCARGSWFWRSGYGYYLDYWGQGTLVTVSS

SEQ ID NO: 347 - protein, 124 aa, Artificial Sequence
  Synthetic SRP1848-F05
EVQLVESGGGLVQPGGSLRLSCAASGFNISKSSIHWVRQAPGKGLEWVGEIYPNDGSTDYADSVKGRFTISADTSKNTAYLQMNSLRAEDTAVYYCARGSWAWRSGYSYFLDYWGQGTLVTVSS

SEQ ID NO: 348 - protein, 124 aa, Artificial Sequence
  Synthetic SRP1848-F06
EVQLVESGGGLVQPGGSLRLSCAASGFNISLSYIHWVRQAPGKGLEWVGGISPIDGNTDYADSVKGRFTISADTSKNTAYLQMNSLRAEDTAVYYCARGFWAWRSGYGYYLDYWGQGTLVTVSS

SEQ ID NO: 349 - protein, 124 aa, Artificial Sequence
  Synthetic SRP1848-F07
EVQLVESGGGLVQPGGSLRLSCAASGFNISNHSIHWVRQAPGKGLEWVGEIYPNDGITDYADSVKGRFTISADTSKNTAYLQMNSLSAEDTAVYYCARGSWDWRSGYSYYLDYWGQGTLVTVSS

SEQ ID NO: 350 - protein, 124 aa, Artificial Sequence
  Synthetic SRP1848-F08
EVQLVESGGGLVQPGGSLRLSCAAGGFNISNHSIHWVRQAPGKGVEWVGEIYPVDGITDYADSVKGRFTISADTSKNTAYLRMNSLRAEDTAVYYCARGSWYWQSGYSYYLDYWGQGTLVTVSS

SEQ ID NO: 351 - protein, 124 aa, Artificial Sequence
  Synthetic SRP1848-F09
EVQLVESGGGLVQPGGSLRLSCAASGFNISNHYIHWVRQAPGKGLEWVGGISPLDGYTDYADSVKGRFTISADTSKNTAYLQMNSLRAEDTAVYYCARGAWSWRSGYGYYIDYWGQGTLVTVSS

SEQ ID NO: 352 - protein, 124 aa, Artificial Sequence
  Synthetic SRP1848-F10
EVQLVESGGGLVQPGGSLRLSCAASGFNISNNSIHWVRQAPGKGLEWVGSIFPNDGYTDYADSVKGRFTISADTSKNTAYLQMNSLRAEDTAVYYCARGSWFWRSGYGYYLDYWGQGTLVTVSS

SEQ ID NO: 353 - protein, 124 aa, Artificial Sequence
  Synthetic SRP1848-F11
EVQLVESGGGLVQPGGSLRLSCAASGFNISNNYIHWVRQAPGKGLEWVGGITPIDGNTDYADSVKGRFTISADTSMNTAYLQMNSLRAEDTAVYYCARGSWYWRAGYGYYLDYWGQGALVTVSS

SEQ ID NO: 354 - protein, 124 aa, Artificial Sequence
  Synthetic SRP1848-G01
EVQLVESGGGLVQPGGSLRLSCAASGFNISRHSIHWVRQAPGKGLEWVGWIAPNDGSTDYADSVKGRFTISADTSKNTAYLQMNSLRAEDTAVYYCARGSWAWRSGYSYFLDYWGQGTLVTVSS

SEQ ID NO: 355 - protein, 124 aa, Artificial Sequence
  Synthetic SRP1848-G03
EVQLVESGGGLVQPGGSLRLSCAASGFNISTYYIHWVRQAPGKGLEWVGGITPSDGFTDYADSVKGRSTISADTSKNTAYLQMNSLRAEDTAVYYCARGSWSWPSGHGYFLDYWGQGTLVTVSS

SEQ ID NO: 356 - protein, 120 aa, Artificial Sequence
  Synthetic SRP1848-G04
EVQLVESGGGLVQPGGSLRLSCAASGFNIHSTDIHWVRQAPGKGLEWVAYITPAGGATFYADSVKGRFTISADTSKNTAYLQMNSLRAEDTAVYYCARYPYWFAGYMDYWGQGTLVTVSS

SEQ ID NO: 357 - protein, 120 aa, Artificial Sequence
  Synthetic SRP1848-G06
EVQLVESGGGLVQPGGSLRLSCAASGFNIRSTDIHWVRQAPGKGLEWVAYITPAGGATYYADNVKGRFTISADTSKNTAYLQMNSLRAEDTAVYYCARQPYWFAGYMDYWGQGTLVTVSS

SEQ ID NO: 358 - protein, 120 aa, Artificial Sequence
  Synthetic SRP1848-G07
EVQLVESGGGLVQPGGSLRLSCAASGFNIHSTDIHWVRQAPGKGLEWVAYITPAGGATWYADSVKGRFTISADTSKNTAYLQMNSLRAEDTAVYYCARYPFWFAGYMDYWGQGTLVTVSS

SEQ ID NO: 359 - protein, 120 aa, Artificial Sequence
  Synthetic SRP1848-G09
EVQLVESGGGLVQPGGSLRLSCAASGFNIRGTDIHWVRQAPGKGLEWVAYITPAGGATFYADSVKGRFTISADTSKNTAYLQMNSLRAEDTAVYYCARHEYWFSGYMDYWGQGTLVTVSS

SEQ ID NO: 360 - protein, 120 aa, Artificial Sequence
  Synthetic SRP1848-G10
EVQLVESGGGLVQPGSSLRLSCAASGFNIRSTDIHWVRQAPGKGLEWVAYITPAGGATYYADSVKGRFTISADTSKNTAYLQMNSLRAEDTAVYYCARYPYWFAGYIDYWGQGTLVTVSS

SEQ ID NO: 361 - protein, 120 aa, Artificial Sequence
  Synthetic SRP1848-G11
EVQLVESGGGLVQPGGSLRLSCAASGFNISSTDIHWVRQAPGKGLEWVAYITPAGGATWYADSVKGRFTISADTSKNTAYLQMNSLRAEDTAVYYCARYPYWFSGYMDYWGQGTLVTVSS

SEQ ID NO: 362 - protein, 124 aa, Artificial Sequence
  Synthetic SRP1848-H01
EVQLVESGGGLVQPGGSLRLSCAASGFNIRTQSIHWVRQAPGKGLEWIGDIFPIDGITDYADSVKGRFTISADTSKNTAYLQMNSLRAEDTAVYYCARGSWSWPSGMDYYLDYWGQGTLVTVSS

SEQ ID NO: 363 - protein, 126 aa, Artificial Sequence
  Synthetic SRP2060-E10
EVQLLESGGGLVQPGGSLRLSCAFSGFSLSTFGMGVGWVRQAPGKGLEWVSHIWWDDDKYYHPALKGRFTISKDNSKNTVYLQMNSLRAEDTAVYYCGRNHFPHYYGSSHWYFNVWGQGTTVTVSS

SEQ ID NO: 364 - protein, 126 aa, Artificial Sequence
  Synthetic SRP2060-E05
EVQLLESGGGLVQPGGSLRLSCAFSGFSLSTFGMGVGWVRQAPGKGLEWVSHIWWDDDKYYHPALKGRFTVSKDNSKNTVYLQMNSLRAEDTAVYYCGRNHFPHYYGSSHWYFNVWGQGTTVTVSS

SEQ ID NO: 365 - protein, 126 aa, Artificial Sequence
  Synthetic SRP2060-B01
EVQLLESGGGLVQPGGSLRLSCALSGFSLSTFGMGVGWVRQATGKGLEWVSHIWWDDDKYYHPALKGRFTISKDNSKNTVHLQMNSLRAEDTAVYYCGRNHFPHYYGSSHWYFNVWGQGTTVTVSS

SEQ ID NO: 366 - protein, 126 aa, Artificial Sequence
  Synthetic SRP2060-A06
EVQLLESGGGLVQPGGSLRLSCAFSGFSLSTFGMGVGWVRQAPGKGLEWVGHIWWDDDKYYYPALKGRFTISKDNSKNTVYLQMNSLRAEDTAVYYCGRNHFPHYYGSSHWYFDVWGQGTTVTVSS

SEQ ID NO: 367 - protein, 107 aa, Artificial Sequence [sequence omitted]

SEQ ID NO: 368 - protein, 107 aa, Artificial Sequence
  Synthetic H6D1-LC4
EIVMTQSPATLSLSPGERATLSCKASQDINSYLSWYQQKPGQAPRLLIYRANRLVDGIPARFSGSGSGTDYTLTISSLEPEDFAVYYCLQYDEFPYTFGGGTKVEIK

SEQ ID NO: 369 - protein, 107 aa, Artificial Sequence
  Synthetic H6D1-LC5
DIQMTQSPSTLSASVGDRVTITCKASQDINSYLSWYQQKPGKAPKLLIYRANRLVDGVPSRFSGSGSGTEFTLTISSLQPDDFATYYCLQYDEFPYTFGGGTKVEIK

SEQ ID NO: 370 - protein, 330 aa, Homo sapiens
  misc_feature (1)..(330): Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 107 aa, Homo sapiens [sequence omitted]

SEQ ID NO: 372 - protein, 323 aa, Mus musculus
  misc_feature (1)..(323): Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 373 - protein, 107 aa, Mus musculus
  misc_feature (1)..(107): Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 374 - protein, 108 aa, Artificial Sequence
  Synthetic Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 106 aa, Artificial Sequence
  Synthetic Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 376 - protein, 20 aa, Artificial Sequence
  Synthetic FlagHis Tag
GSGDYKDDDDKGSGHHHHHH

SEQ ID NO: 377 - protein, 15 aa, Artificial Sequence
  Synthetic Linker
GGGGSGGGGSGGGGS

SEQ ID NO: 378 - protein, 11 aa, Artificial Sequence
  Synthetic Linker
AAGSDQEPKSS

SEQ ID NO: 379 - protein, 247 aa, Artificial Sequence
  Synthetic 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 380 - protein, 247 aa, Artificial Sequence
  Synthetic 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSS

SEQ ID NO: 381 - protein, 485 aa, Artificial Sequence
  Synthetic 1848-B10-VH-(G4S)3-VL
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 485 aa, Artificial Sequence
  Synthetic 1848-B10-VL-(G4S)3-VH
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 30 aa, Artificial Sequence
  Synthetic linker
  misc_feature (16)..(30): “GGGGS” may or may not be present
GGGGSGGGGSGGGGSGGGGSGGGGSGGGGS